(19) United States
(12) Patent Application Publication
(10) Pub. No.: US 2025/0235397 A1
(43) Pub. Date: Jul. 24, 2025

(54) COMPOSITIONS AND METHODS OF USE THEREOF FOR TREATING AND PREVENTING HALITOSIS

(71) Applicant: Imvela Corp., Brooklyn, NY (US)

(72) Inventors: Raphaël Turcotte, Brooklyn, NY (US); Ravi Sheth, Brooklyn, NY (US)

(73) Assignee: Imvela Corp., Brooklyn, NY (US)

(21) Appl. No.: 19/028,696

(22) Filed: Jan. 17, 2025

Related U.S. Application Data
(63) Continuation-in-part of application No. 18/790,185, filed on Jul. 31, 2024.
(60) Provisional application No. 63/622,511, filed on Jan. 18, 2024.

(51) Int. Cl. A61K 8/99; A61Q 11/00

(52) U.S. Cl. CPC A61K 8/99; A61Q 11/00

(57) ABSTRACT

Some aspects of the disclosure relate to compositions comprising fermentates of bacterial strains capable of reducing volatile sulfur compounds (VSCs) in an environment, nutritional supplements, dental supplements, and food products containing such fermentates, and methods of use thereof.

### RELATED APPLICATIONS

This application is a continuation-in-part of U.S. application Ser. No. 18/790,185, filed Jul. 31, 2024, which claims the benefit under 35 U.S.C. § 119 (e) of U.S. Provisional Application No. 63/622,511, filed Jan. 18, 2024, the contents of each of which are incorporated by reference herein in their entirety.

### REFERENCE TO AN ELECTRONIC SEQUENCE LISTING

The contents of the electronic sequence listing (1055870005US02-SEQ-NTJ.xml; Size: 31,893,292 bytes; and Date of Creation: Jan. 17, 2025) are herein incorporated by reference in their entirety.

### BACKGROUND

Halitosis, commonly known as bad breath, is an oral health issue that affects several mammalian species, including humans and dogs. Halitosis is characterized by the buildup of compounds within the oral cavity, which emit a strong, offensive odor. The main malodorous compounds are volatile sulfur compounds (VSCs). These compounds are byproducts of bacterial metabolism. VSCs include hydrogen sulfide, methyl mercaptan, and dimethyl sulfide, all of which give off strong and unpleasant odors.

### SUMMARY

Some aspects relate to compositions comprising fermentate(s) of bacterial strains. Some aspects relate to methods of using compositions comprising fermentate(s) of bacterial strains. Such compositions and methods are based, at least in part, on the recognition that bacterial strains and fermentates thereof are able to effectively decrease volatile sulfur compounds (VSCs), which are associated with halitosis, and disrupt biofilms of VSC-producing microorganisms, thereby inhibiting continued VSC production by such microbes. While such fermentates alone are useful for reduction of VSC abundance, compositions may also comprise additional fermentates of other bacterial strains useful in treating or preventing halitosis.

For example, a composition described herein may comprise a fermentate of a bacterial strain that disrupts a biofilm of a canine oral microbe (e.g., Neisseria canis), thereby reducing the abundance of canine oral microbes and reducing ongoing VSC production by such microbes. A composition described herein may comprise a fermentate of a bacterial strain that produces an antioxidant, which can absorb free radicals of VSCs, thereby mitigating VSCs' contribution to malodor. A composition described herein may comprise a fermentate of a bacterial strain that enhances the function of one or more other bacterial strains or fermentates, such as their ability to reduce VSC abundance, inhibit VSC production, disrupt biofilms, and/or absorb free radicals of VSCs. A composition described herein may further comprise one or more additional components, such as vitamins, organic acids, polyphenols, indole-containing compounds, and phenols. In addition to the benefits of organic acids in reducing the oral microenvironment pH, thereby hindering VSC production and/or growth of VSC-producing microorganisms, such additional components may enhance the VSC-reducing ability of bacterial strains present in a composition.

Accordingly, some aspects relate to a method of treating one or more symptoms of halitosis in a subject in need thereof, the method comprising orally administering to the subject a nutritional supplement, dental supplement, or food product comprising a bacterial fermentate mixture, the bacterial fermentate mixture comprising: (i) a first fermentate of a first bacterial strain comprising a 16S rDNA sequence with at least 97% sequence identity to the nucleotide sequence of any one of SEQ ID NOs: 1-4; and (ii) a second fermentate of a second bacterial strain comprising a 16S rDNA sequence with at least 97% sequence identity to the nucleotide sequence of SEQ ID NO: 5.

In some embodiments, the bacterial fermentate mixture comprises: (i) a first fermentate of a first bacterial strain comprising a 16S rDNA sequence with at least 97% sequence identity to the nucleotide sequence of SEQ ID NO: 1; and (ii) a second fermentate of a second bacterial strain comprising a 16S rDNA sequence with at least 97% sequence identity to the nucleotide sequence of SEQ ID NO: 5.

In some embodiments, the bacterial fermentate mixture comprises: (i) a first fermentate of a first bacterial strain comprising a 16S rDNA sequence with at least 99% sequence identity to the nucleotide sequence of any one of SEQ ID NOs: 1-4; and (ii) a second fermentate of a second bacterial strain comprising a 16S rDNA sequence with at least 99% sequence identity to the nucleotide sequence of SEQ ID NO: 5.

In some embodiments, the bacterial fermentate mixture comprises: (i) a first fermentate of a first bacterial strain comprising a 16S rDNA sequence with at least 99% sequence identity to the nucleotide sequence of SEQ ID NO: 1; and (ii) a second fermentate of a second bacterial strain comprising a 16S rDNA sequence with at least 99% sequence identity to the nucleotide sequence of SEQ ID NO: 5.

In some embodiments, the bacterial fermentate mixture consists of: (i) a first fermentate of a first bacterial strain comprising a 16S rDNA sequence with at least 97% sequence identity to the nucleotide sequence of any one of SEQ ID NOs: 1-4; and (ii) a second fermentate of a second bacterial strain comprising a 16S rDNA sequence with at least 97% sequence identity to the nucleotide sequence of SEQ ID NO: 5.

In some embodiments, the bacterial fermentate mixture consists of: (i) a first fermentate of a first bacterial strain comprising a 16S rDNA sequence with at least 97% sequence identity to the nucleotide sequence of SEQ ID NO: 1; and (ii) a second fermentate of a second bacterial strain comprising a 16S rDNA sequence with at least 97% sequence identity to the nucleotide sequence of SEQ ID NO: 5.

In some embodiments, the bacterial fermentate mixture consists of: (i) a first fermentate of a first bacterial strain comprising a 16S rDNA sequence with at least 99% sequence identity to the nucleotide sequence of any one of SEQ ID NOs: 1-4; and (ii) a second fermentate of a second bacterial strain comprising a 16S rDNA sequence with at least 99% sequence identity to the nucleotide sequence of SEQ ID NO: 5.

In some embodiments, the bacterial fermentate mixture consists of: (i) a first fermentate of a first bacterial strain comprising a 16S rDNA sequence with at least 99% sequence identity to the nucleotide sequence of SEQ ID NO: 1; and (ii) a second fermentate of a second bacterial strain comprising a 16S rDNA sequence with at least 99% sequence identity to the nucleotide sequence of SEQ ID NO: 5.

In some embodiments, the first fermentate disrupts a biofilm of one or more canine oral microbes.

In some embodiments, the second fermentate reduces abundance of one or more volatile sulfur compounds (VSCs).

In some embodiments, the nutritional supplement, dental supplement, or food product reduces abundance of one or more VSCs in an oral microenvironment in the subject.

In some embodiments, the wherein the nutritional supplement, dental supplement, or food product reduces, in an oral microenvironment of the subject, abundance of one or more microorganisms that produce one or more VSCs.

In some embodiments, the subject is a dog or cat.

In some embodiments, the subject is a dog.

In some embodiments, each of the fermentates is present in the nutritional supplement, dental supplement, or food product at an amount of 1 to 500 mg.

In some embodiments, the bacterial fermentate mixture is present in the nutritional supplement, dental supplement, or food product at an incorporation rate of 0.01% to 10% w/w.

In some embodiments, the nutritional supplement, dental supplement, or food product is in the form of a gelatinized starch matrix, extruded or injection-molded dental stick, extruded or injection-molded dental chew, chew having a soft interior, powder meal topper, water additive, dry kibble or food, wet food, frozen food, liquid spray, peanut butter spread, or soft jerky chew.

Some aspects relate to a method of treating one or more symptoms of halitosis in a dog in need thereof, the method comprising orally administering to the dog a nutritional supplement, dental supplement, or food product comprising a bacterial fermentate mixture, the bacterial fermentate mixture comprising: (i) a first fermentate of a first bacterial strain comprising a 16S rDNA sequence with at least 99% sequence identity to the nucleotide sequence of SEQ ID NO: 1; and (ii) a second fermentate of a second bacterial strain comprising a 16S rDNA sequence with at least 99% sequence identity to the nucleotide sequence of SEQ ID NO: 5. In some embodiments, the first fermentate disrupts a biofilm of one or more canine oral microbes. In some embodiments, the second fermentate reduces abundance of one or more volatile sulfur compounds (VSCs).

some aspects of the present disclosure relate to a postbiotic composition comprising (a) a fermentate of a first bacterial strain comprising a 16S rDNA sequence with at least 97% sequence identity to the nucleic acid sequence of any one of SEQ ID NOs: 1-4; and (b) one or more additional fermentates, wherein each of the one or more additional fermentates are of a bacterial strain comprising a 16S rDNA sequence with at least 97% sequence identity to the nucleic acid sequence of any one of SEQ ID NO: 5-9.

In some embodiments, the bacterial strain of (a) is capable of disrupting a biofilm of one or more canine oral microbes. In some embodiments, the bacterial strain of (b) is capable of (i) producing one or more compounds able to reduce the abundance of one or more volatile sulfur compounds (VSCs); (ii) producing one or more antioxidants; (iii) enhancing a function of one or more bacterial strains. In some embodiments, the antioxidant is selected from the group consisting of a vitamin, an organic acid, a polyphenol, an indole-containing compound, and a phenol. In some embodiments, the antioxidant is reactive to the ferric ion-reducing antioxidant power assay.

Some aspects relate to a postbiotic composition comprising (a) a first fermentate of a first bacterial strain comprising a 16S rDNA sequence with at least 97% sequence identity to the nucleic acid sequence of any one of SEQ ID NOs: 1-4; and (b) a second fermentate of a second bacterial strain comprising a 16S rDNA sequence with at least 97% sequence identity to the nucleic acid sequence of SEQ ID NO: 5. In some embodiments, the composition further comprises (c) a third fermentate of a third bacterial strain comprising a 16s rDNA sequence with at least 97% sequence identity to the nucleic acid sequence of any one of SEQ ID NOs: 6-9. In some embodiments, (a) the first bacterial strain comprises a 16S rDNA sequence with at least 97% sequence identity to the nucleic acid sequence of SEQ ID NO: 1; (b) the second bacterial strain comprises a 16S rDNA sequence with at least 97% sequence identity to the nucleic acid sequence of SEQ ID NO: 5; and (c) the third bacterial strain comprises a 16S rDNA sequence with at least 97% sequence identity to the nucleic acid sequence of SEQ ID NO: 6.

In some embodiments, (a) the first bacterial strain comprises a 16S rDNA sequence with at least 97% sequence identity to the nucleic acid sequence of SEQ ID NO: 1; (b) the second bacterial strain comprises a 16S rDNA sequence with at least 97% sequence identity to the nucleic acid sequence of SEQ ID NO: 5; and (c) the third bacterial strain comprises a 16S rDNA sequence with at least 97% sequence identity to the nucleic acid sequence of SEQ ID NO: 9.

Some aspects relate to a postbiotic composition comprising (a) a first fermentate of a first bacterial strain comprising a 16S rDNA sequence with at least 97% sequence identity to the nucleic acid sequence of any one of SEQ ID NOs: 1-4; and (b) a second fermentate of a second bacterial strain comprising a 16S rDNA sequence with at least 97% sequence identity to the nucleic acid sequence of any one of SEQ ID NOs: 6-9. In some embodiments, the composition further comprises a third fermentate of a third bacterial strain comprising a 16s rDNA sequence with at least 97% sequence identity to the nucleic acid sequence of SEQ ID NO: 5.

In some embodiments, (a) the first bacterial strain comprises a 16S rDNA sequence with at least 97% sequence identity to the nucleic acid sequence of SEQ ID NO: 1; (b) the second bacterial strain comprises a 16S rDNA sequence with at least 97% sequence identity to the nucleic acid sequence of SEQ ID NO: 5; and (c) the third bacterial strain comprises a 16S rDNA sequence with at least 97% sequence identity to the nucleic acid sequence of SEQ ID NO: 6.

Some aspects relate to a postbiotic composition comprising: (a) a fermentate of a bacterial strain that disrupts an oral biofilm; and at least one of: (b)(i) a fermentate of a bacterial strain that produces one or more compounds able to reduce the abundance of one or more volatile sulfur compounds (VSCs), (b)(ii) a fermentate of a bacterial strain that produces one or more antioxidants; and (b)(iii) a fermentate of a bacterial strain that enhances a function of one or more bacterial strains.

In some embodiments, the bacterial strain of (a) is selected from the group consisting of Bacillus subtilis, Bacillus amyloliquefaciens, Bacillus licheniformis, and Bacillus pumilus. In some embodiments, the bacterial strain of (b)(i) is Pediococcus pentosaceus. In some embodiments, the bacterial strain of (b)(i) is selected from the group consisting of Pediococcus pentosaceus, Bacillus subtilis, and Lactiplantibacillus plantarum. In some embodiments, the bacterial strain of (b)(ii) is selected from the group consisting of Lactiplantibacillus plantarum, Lactobacillus delbrueckii, and Limosilactobacillus fermentum. In some embodiments, the bacterial strain of (b)(ii) is selected from the group consisting of Pediococcus pentosaceus, Lactiplantibacillus plantarum, Lactobacillus delbrueckii, and Limosilactobacillus fermentum.

In some embodiments, the bacterial strain of (iii) is selected from the group consisting of Bacillus subtilis and Lactiplantibacillus plantarum.

In some embodiments, composition reduces the abundance of one or more volatile sulfur compounds (VSCs) in an oral cavity of a subject. In some embodiments, the composition reduces the abundance of one or more volatile sulfur compound (VSC)-producing microorganisms in a subject. In some embodiments, the one or more VSC-producing microorganisms are selected from the group consisting of Fusobacterium nucleatum, Neisseria canis, Bergyella zoohelcum, Prevotella intermedia, Porphyromonas canigingivalis, Porphyromonas gingivalis, Tannerella forsythia, and Treponema denticola.

In some embodiments, the one or more VSCs are selected from the group consisting of hydrogen sulfide, methyl mercaptan, and dimethyl sulfide. In some embodiments, the composition reduces the level of gum inflammation, gingivitis, plaque, tartar, and/or dental calculus accumulation in an oral cavity of a subject. In some embodiments, the composition treats or prevents one or more symptom of halitosis in a subject. In some embodiments, each of the fermentates are in powdered form. In some embodiments, each of the fermentates are lyophilized or spray-dried.

In some embodiments, each fermentate is present in an amount of 1 to 500 mg. In some embodiments, the composition comprises a total amount of fermentate in an amount of 1 to 500 mg.

In some embodiments, the composition further comprises a carrier. In some embodiments, the carrier is maltodextrin. In some embodiments, the carrier is a tapioca-based maltodextrin.

Some aspects relate to a nutritional supplement, dental supplement, or food product comprising the composition.

Some aspects relate to a nutritional supplement, dental supplement, or food product comprising the composition at an incorporation rate of 0.01% to 10% [w/w]. Some aspects relate to a nutritional supplement, dental supplement, or food product comprising the composition at an incorporation rate of 0.1% to 2% [w/w].

In some embodiments, the nutritional supplement is a canine nutritional supplement. In some embodiments, the dental supplement is a canine nutritional supplement. In some embodiments, the nutritional supplement, dental supplement, or food product comprises a meat or animal-derived material. In some embodiments, the meat or animal-derived material is beef, chicken, eggs, turkey, lamb, fish, blood marrow, and/or bone marrow.

In some embodiments, the nutritional supplement, dental supplement, or food product further comprises a grain. In some embodiments, the grain is wheat, corn, rice, oats, and/or barley.

In some embodiments, the nutritional supplement, dental supplement, or food product further comprises a fiber. In some embodiments, the fiber is sugar beet pulp, chicory pulp, chicory, coconut endosperm fiber, wheat fiber, fructooligosaccharides, galactooligosaccharides, and/or inulin.

In some embodiments, the nutritional supplement, dental supplement, or food product further comprises algae. In some embodiments, the algae is selected from the group consisting of Ascophyllum nodosum, Spirulina, Chlorella, Ulva lactuca, Laminaria digitata and Fucus vesiculosus.

In some embodiments, the nutritional supplement, dental supplement, or food product further comprises a gelatinized starch matrix.

In some embodiments, the nutritional supplement, dental supplement, or food product reduces inflammation in a subject. In some embodiments, the nutritional supplement, dental supplement, or food product treats or prevents a symptom associated with halitosis in a subject. In some embodiments, the dental supplement is an extruded or injection-molded dental stick. In some embodiments, the dental supplement is an extruded or injection-molded dental chew. In some embodiments, the nutritional supplement or dental supplement is a chew having a soft interior.

In some embodiments, the nutritional supplement is a meal topper. In some embodiments, the nutritional supplement is a water additive. In some embodiments, the food product is an extruded kibble. In some embodiments, the food product is a fresh food. In some embodiments, the nutritional supplement is a liquid spray. In some embodiments, the nutritional supplement or food product is peanut butter spread. In some embodiments, the nutritional supplement, dental supplement, or food product is soft jerky chew.

Some aspects relate to an oral care product comprising a composition described herein.

In some embodiments, the oral care product comprises the composition at an incorporation rate of 0.01% to 10% [w/w]. In some embodiments, the oral care product comprises the composition at an incorporation rate of 0.1% to 2% [w/w]. In some embodiments, the oral care product treats or prevents a symptom associated with halitosis in a subject.

In some embodiments, the oral care product is a toothpaste. In some embodiments, the oral care product is a mouthwash. In some embodiments, the oral care product is a breath mint. In some embodiments, the oral care product is a chewing gum.

Some aspects relate to a method comprising a composition, nutritional supplement, dental supplement, or food product to a subject. In some embodiments, the administration is oral administration (e.g., consumption of the composition, nutritional supplement, dental supplement, or food product) by the subject. In some embodiments, the subject has halitosis.

In some embodiments, an oral microenvironment of the subject comprises one or more bacterial strains selected from Porphyromonas gingivalis, Prevotella intermedia, Porphyromonas canigingivalis, Tannerella forsythia, Treponema denticola, Fusobacterium nucleatum, Neisseria canis, and Bergeyella zoohelcum.

In some embodiments, administration of the composition, nutritional supplement, dental supplement, or food product: (i) reduces the abundance of one or more VSCs in an oral microenvironment of the subject; (ii) reduces inflammation in the oral microenvironment of the subject; (iii) reduces the abundance of gingivitis, plaque, tartar, and/or dental calculus in the subject; (iv) reduces the abundance of one or more microorganisms associated with the production of VSCs; (v) reduces one or more symptom associated with halitosis; and/or (vi) improves oral health of the subject.

In some embodiments, the subject is a carnivore. In some embodiments, the subject is a mammal. In some embodiments, the subject is a domesticated animal. In some embodiments, the subject is a bovine, swine, llama, alpaca, sheep, or goat. In some embodiments, the subject is a dog, cat, rabbit, guinea pig, hamster, or ferret. In some embodiments, the subject is a dog. In some embodiments, the subject is a human.

### BRIEF DESCRIPTION OF THE DRAWINGS

The accompanying drawings, which constitute a part of this specification, illustrate several embodiments of the disclosure and together with the description, serve to explain the principles of the disclosure.

FIG. 1 shows effects of fermentate compositions on VSC production Fusobacterium nucleatum, a known canine oral microbe.

FIG. 2 shows dose response of fermentate compositions on reduction of VSC production by F. nucleatum. “Dose A” corresponds to a 0.3× dose; “Dose B” corresponds to a 1× dose; and “Dose C” corresponds to 3× dose.

FIG. 3 shows disruption of biofilm of canine oral microbes by fermentate composition.

FIG. 4 shows disruption of biofilm of canine oral microbes by separate individual fermentates of Bacillus subtilis. Biofilm area after treatment with each fermentate is normalized to area following treatment with negative control (media only).

FIG. 5 shows VSC levels on Days 0 and 7 post-administration for different test groups. At each day, left bars represent Placebo, and right bars represent Canine Oral Health Postbiotic (COHP) treatment group.

FIGS. 6A and 6B show Operational Taxonomic Units (OTUs) with significant log 2 fold changes in Placebo (FIG. 6A) and COHP (FIG. 6B) treatment groups.

FIG. 7 shows normalized VSC levels from an in vitro assay after treatment with COHP or control compositions. Bars indicate the average percentage of VSCs remaining after treatment, across replicates measured in the experiment. Error bars represent the standard deviation.

FIG. 8 shows biofilm disruption in an in vitro assay after treatment with COHP or control compositions. Each point is a technical replicate. Bars indicate the average and standard deviation.

FIG. 9 shows oral VSC levels in canine subjects in a placebo-controlled clinical study evaluating the effects of COHP. The average and standard deviation of VSC measurements on each sampling day 0 (baseline), 7, and 14 is shown.

FIG. 10A shows taxa of the oral microbiome that changed in subjects receiving control (top panel) or canine oral health postbiotic (COHP) composition (lower panel), based on p-values <0.1 (FDR corrected via BH method). FIG. 10B shows diversity measurements, in terms of observed α diversity (left), evenness (middle), and Shannon diversity index (right panel). Within each group, box-and-whisker plots show data on days 0 (baseline), 7, and 14. Diversity analyses were performed using DESeq2.

FIG. 11 shows the correlation between VSC levels and perceived malodor. Points represent measurements of a single participant at a single time point. Black line represents the best fit line to the data, indicating a positive correlation between the two measurements.

### DETAILED DESCRIPTION

Aspects of the disclosure relate to compositions, nutritional supplements, dental supplements, food product, and/or oral care products comprising fermentates of one or more bacterial strains that reduce levels of volatile sulfur compounds (VSCs) in environment, inhibit VSC production by other microorganisms, and/or disrupt biofilms of VSC-producing microorganisms, and methods of use thereof. Without wishing to be bound by theory, the compositions comprising fermentates described herein are useful for reducing VSC abundance, which is associated with halitosis. Fermentates of other bacterial strains may further enhance the VSC-reducing and/or halitosis-alleviating properties of such compositions, such as by inhibiting VSC production, producing or providing antioxidants that absorb free radicals of VSCs, disrupting biofilms of VSC-producing microbes, and/or reducing or slowing accumulation of plaque, tartar, or dental calculus.

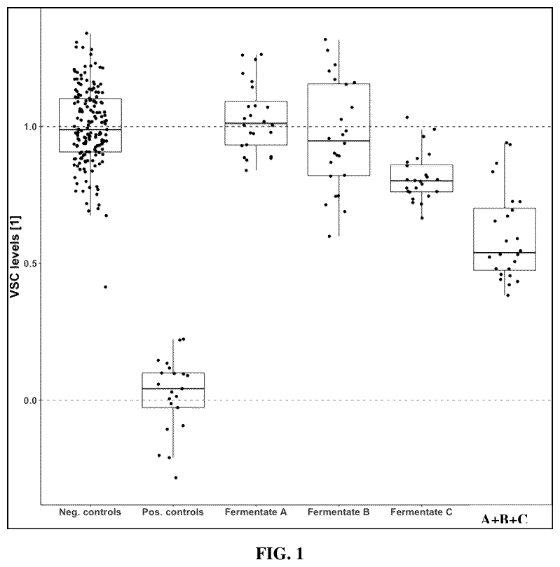

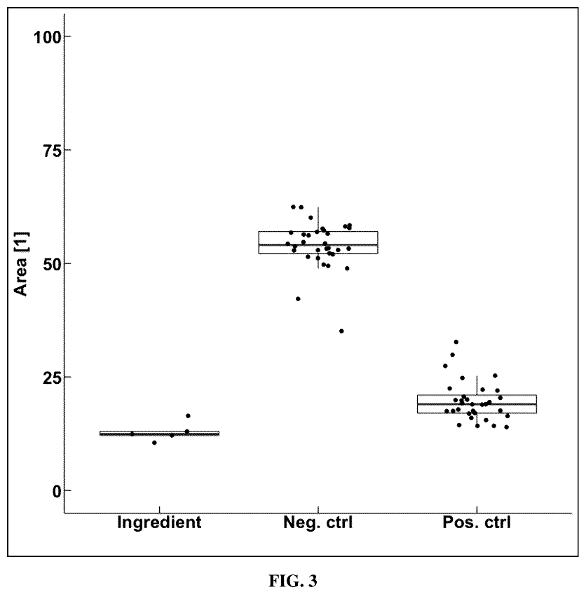

Compositions comprising fermentates of microbes such as Bacillus subtilis, Pediococcus pentosaceus, and/or Lactobacillus plantarum (among others) were found to be effective in reducing the abundance of VSCs produced by microbes associated with halitosis (e.g., Fusobacterium nucleatum), with synergistic reductions being observed when fermentates from multiple species were combined (FIG. 1). Furthermore, fermentates disrupted biofilms of other canine oral microbes associated with halitosis (e.g., Neisseria canis and Bergeyella zoohelcum) (FIG. 3). Thus, compositions containing fermentates may be useful, for example, in mitigating growth of oral microbes associated with VSC production and other consequences of unmediated halitosis, such as plaque and dental calculus accumulation.

Compositions comprising bacterial fermentates may be in any suitable form, such as a dry powder that may be combined with other compositions (e.g., combination with food prior to a meal) or in liquid form that may be added to water or combined with other compositions (e.g., combined with or applied to food prior to a meal). Compositions comprising bacterial fermentates may also be present as part of another composition, such as a pill, capsule, tablet, chewable matrix, nutritional supplement, dental supplement, food product, and/or oral care product.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### Bacterial Strains and Fermentates Thereof

Some aspects relate to compositions (and/or nutritional supplements, dental supplements, food product, and/or oral care products comprising a composition) comprising (a) a fermentate of a first bacterial strain comprising a 16S rDNA sequence with at least 97% sequence identity to a nucleotide sequence of any one of SEQ ID NOs: 1-4; and (b) one or more additional fermentates, each being a fermentate of a bacterial strain comprising a 16S rDNA sequence with at least 97% sequence identity to a nucleotide sequence of any one of SEQ ID NOs: 5-9.

Some aspects relate to compositions (and/or nutritional supplements, dental supplements, food product, and/or oral care products comprising a composition) comprising (a) a fermentate of a first bacterial strain belonging to a species selected from the group consisting of Bacillus subtilis, Bacillus amyloliquefaciens, Bacillus licheniformis, and Bacillus pumilus; and one or more additional fermentates, each being a fermentate of a bacterial strain belonging to a species selected from the group consisting of Pediococcus pentosaceus, Lactiplantibacillus plantarum, Lactobacillus delbrueckii, Limosilactobacillus fermentum, and Bacillus subtilis, where the Bacillus subtilis strain producing additional fermentate is different from the Bacillus subtilis producing the first fermentate.

Some aspects relate to compositions (and/or nutritional supplements, dental supplements, food product, and/or oral care products comprising a composition) comprising (a) a first fermentate of a first bacterial strain comprising a 16S rDNA sequence with at least 97% sequence identity to a nucleotide sequence of any one of SEQ ID NOs: 1-4; and (b) a second fermentate of a second bacterial strain comprising a 16S rDNA sequence comprising a 16S rDNA sequence with at least 97% sequence identity to a nucleotide sequence of SEQ ID NO: 5. In some embodiments, the composition comprises (a) a first fermentate of a first bacterial strain comprising a 16S rDNA sequence with at least 97% sequence identity to a nucleotide sequence of SEQ ID NO: 1. In some embodiments, the composition comprises (a) a first fermentate of a first bacterial strain comprising a 16S rDNA sequence with at least 97% sequence identity to a nucleotide sequence of SEQ ID NO: 2. In some embodiments, the composition comprises (a) a first fermentate of a first bacterial strain comprising a 16S rDNA sequence with at least 97% sequence identity to a nucleotide sequence of SEQ ID NO: 3. In some embodiments, the composition comprises (a) a first fermentate of a first bacterial strain comprising a 16S rDNA sequence with at least 97% sequence identity to a nucleotide sequence of SEQ ID NO: 4.

Some aspects relate to compositions (and/or nutritional supplements, dental supplements, food product, and/or oral care products comprising a composition) comprising (a) a first fermentate of a first bacterial strain belonging to a species selected from the group consisting of Bacillus subtilis, Bacillus amyloliquefaciens, Bacillus licheniformis, and Bacillus pumilus; and (b) a second fermentate of a second bacterial strain belonging to Pediococcus pentosaceus. In some embodiments, the composition comprises a first fermentate of a first bacterial strain belonging to Bacillus subtilis. In some embodiments, the composition comprises a first fermentate of a first bacterial strain belonging to Bacillus amyloliquefaciens. In some embodiments, the composition comprises a first fermentate of a first bacterial strain belonging to Bacillus licheniformis. In some embodiments, the composition comprises a first fermentate of a first bacterial strain belonging to Bacillus pumilus.

In some embodiments, the composition comprises (a) a first fermentate of a first bacterial strain comprising a 16S rDNA sequence with at least 97% sequence identity to a nucleotide sequence of SEQ ID NO: 1; (b) a second fermentate of a second bacterial strain comprising a 16S rDNA sequence comprising a 16S rDNA sequence with at least 97% sequence identity to a nucleotide sequence of SEQ ID NO: 5; and (c) a third fermentate of a third bacterial strain comprising a 16S rDNA sequence comprising a 16S rDNA sequence with at least 97% sequence identity to a nucleotide sequence of SEQ ID NO: 6.

In some embodiments, the composition comprises (a) a first fermentate of a first bacterial strain belonging to Bacillus subtilis; (b) a second fermentate of a second bacterial strain belonging to Pediococcus pentosaceus; and (c) a third fermentate of a third bacterial strain belonging to Lactobacillus plantarum.

In some embodiments, the composition comprises (a) a first fermentate of a first bacterial strain comprising a 16S rDNA sequence with at least 97% sequence identity to a nucleotide sequence of SEQ ID NO: 1; (b) a second fermentate of a second bacterial strain comprising a 16S rDNA sequence comprising a 16S rDNA sequence with at least 97% sequence identity to a nucleotide sequence of SEQ ID NO: 5; and (c) a third fermentate of a third bacterial strain comprising a 16S rDNA sequence comprising a 16S rDNA sequence with at least 97% sequence identity to a nucleotide sequence of SEQ ID NO: 9.

In some embodiments, the composition comprises (a) a first fermentate of a first bacterial strain belonging to Bacillus subtilis; (b) a second fermentate of a second bacterial strain belonging to Pediococcus pentosaceus; and (c) a third fermentate of a third bacterial strain belonging to Bacillus subtilis that is different from the first Bacillus subtilis strain.

Some aspects relate to compositions (and/or nutritional supplements, dental supplements, food product, and/or oral care products comprising a composition) comprising (a) a first fermentate of a first bacterial strain comprising a 16S rDNA sequence with at least 97% sequence identity to a nucleotide sequence of any one of SEQ ID NOs: 1-4; and (b) a second fermentate of a second bacterial strain comprising a 16S rDNA sequence with at least 97% sequence identity to a nucleotide sequence of any one of SEQ ID NOs: 6-9.

Some aspects relate to compositions (and/or nutritional supplements, dental supplements, food product, and/or oral care products comprising a composition) comprising (a) a first fermentate of a first bacterial strain belonging to a species selected from the group consisting of Bacillus subtilis, Bacillus amyloliquefaciens, Bacillus licheniformis, and Bacillus pumilus; and (b) a second fermentate of a second bacterial strain belonging to a species selected from the group consisting of Lactiplantibacillus plantarum, Lactobacillus delbrueckii, Limosilactobacillus fermentum, and Bacillus subtilis, where the Bacillus subtilis strain producing the second fermentate is different from the Bacillus subtilis producing the first fermentate.

In some embodiments, the composition comprises (a) a first fermentate of a first bacterial strain comprising a 16S rDNA sequence with at least 97% sequence identity to a nucleotide sequence of SEQ ID NO: 1, and (b) a second fermentate of a second bacterial strain comprising a 16S rDNA sequence with at least 97% sequence identity to a nucleotide sequence of SEQ ID NO: 6. In some embodiments, the composition comprises (a) a first fermentate of a first bacterial strain comprising a 16S rDNA sequence with at least 97% sequence identity to a nucleotide sequence of SEQ ID NO: 1, and (b) a second fermentate of a second bacterial strain comprising a 16S rDNA sequence with at least 97% sequence identity to a nucleotide sequence of SEQ ID NO: 7. In some embodiments, the composition comprises (a) a first fermentate of a first bacterial strain comprising a 16S rDNA sequence with at least 97% sequence identity to a nucleotide sequence of SEQ ID NO: 1, and (b) a second fermentate of a second bacterial strain comprising a 16S rDNA sequence with at least 97% sequence identity to a nucleotide sequence of SEQ ID NO: 8. In some embodiments, the composition comprises (a) a first fermentate of a first bacterial strain comprising a 16S rDNA sequence with at least 97% sequence identity to a nucleotide sequence of SEQ ID NO: 1, and (b) a second fermentate of a second bacterial strain comprising a 16S rDNA sequence with at least 97% sequence identity to a nucleotide sequence of SEQ ID NO: 9.

In some embodiments, the composition comprises (a) a first fermentate of a first bacterial strain belonging to Bacillus subtilis; and (b) a second fermentate of a second bacterial strain belonging to Lactiplantibacillus plantarum. In some embodiments, the composition comprises (a) a first fermentate of a first bacterial strain belonging to Bacillus subtilis; and (b) a second fermentate of a second bacterial strain belonging to Lactobacillus delbrueckii. In some embodiments, the composition comprises (a) a first fermentate of a first bacterial strain belonging to Bacillus subtilis; and (b) a second fermentate of a second bacterial strain belonging to Limosilactobacillus fermentum. In some embodiments, the composition comprises (a) a first fermentate of a first bacterial strain belonging to Bacillus subtilis; and (b) a second fermentate of a second bacterial strain belonging to Bacillus subtilis that is different from the first Bacillus subtilis strain.

In some embodiments, the composition further comprises a third fermentate of a third bacterial strain comprising a 16S rDNA sequence with at least 97% sequence identity to a nucleotide sequence of SEQ ID NO: 5. In some embodiments, the composition comprises (a) a first fermentate of a first bacterial strain comprising a 16S rDNA sequence with at least 97% sequence identity to a nucleotide sequence of SEQ ID NO: 1; (b) a second fermentate of a second bacterial strain comprising a 16S rDNA sequence comprising a 16S rDNA sequence with at least 97% sequence identity to a nucleotide sequence of SEQ ID NO: 5; and (c) a third fermentate of a third bacterial strain comprising a 16S rDNA sequence comprising a 16S rDNA sequence with at least 97% sequence identity to a nucleotide sequence of SEQ ID NO: 6.

In some embodiments, the composition further comprises a third fermentate of a third bacterial strain belonging to Pediococcus pentosaceus. In some embodiments, the composition comprises (a) a first fermentate of a first bacterial strain belonging to Bacillus subtilis; (b) a second fermentate of a second bacterial strain belonging to Pediococcus pentosaceus; and (c) a third fermentate of a third bacterial strain belonging to Lactiplantibacillus plantarum.

Some aspects relate to compositions (and/or nutritional supplements, dental supplements, food products, and/or oral care products comprising a composition) comprising at least three fermentates: (i) a first fermentate of a bacterial strain comprising a 16S rDNA sequence with at least 97% sequence identity to the nucleotide sequence of any one of SEQ ID NOs: 1-4; (ii) a second fermentate of a bacterial strain comprising a 16S rDNA sequence with at least 97% sequence identity to the nucleotide sequence of any one of SEQ ID NOs: 5-6; and (iii) a third fermentate of a bacterial strain comprising a 16S rDNA sequence with at least 97% sequence identity to the nucleotide sequence of any one of SEQ ID NOs: 6-8. In some embodiments, a composition comprises at least three fermentates: (i) a first fermentate of a bacterial strain comprising a 16S rDNA sequence with at least 99% sequence identity to the nucleotide sequence of any one of SEQ ID NOs: 1-4; (ii) a second fermentate of a bacterial strain comprising a 16S rDNA sequence with at least 99% sequence identity to the nucleotide sequence of any one of SEQ ID NOs: 5-6; and (iii) a third fermentate of a bacterial strain comprising a 16S rDNA sequence with at least 99% sequence identity to the nucleotide sequence of any one of SEQ ID NOs: 6-8. In some embodiments, a composition comprises at least three fermentates: (i) a first fermentate of a bacterial strain belonging to the species Bacillus subtilis, Bacillus amyloliquefaciens, Bacillus licheniformis, or Bacillus pumilus; (ii) a second fermentate of a bacterial strain belonging to the species Pediococcus pentosaceus or Lactiplantibacillus plantarum; and (iii) a third fermentate of a bacterial strain belonging to the species Lactiplantibacillus plantarum, Lactobacillus delbrueckii, or Limosilactobacillus fermentum. The skilled artisan will appreciate that each of the 24 individual combinations formed by one of the 4 options of (i), one of the 2 options of (ii), and one of the 3 options of (iii) is contemplated.

Some aspects relate to compositions (and/or nutritional supplements, dental supplements, food products, and/or oral care products comprising a composition) comprising at least three fermentates: (i) a first fermentate of a bacterial strain comprising a 16S rDNA sequence with at least 97% sequence identity to the nucleotide sequence of any one of SEQ ID NOs: 1-4; (ii) a second fermentate of a bacterial strain comprising a 16S rDNA sequence with at least 97% sequence identity to the nucleotide sequence of any one of SEQ ID NOs: 5-6; and (iii) a third fermentate of a bacterial strain comprising a 16S rDNA sequence with at least 97% sequence identity to the nucleotide sequence of SEQ ID NO: 9. In some embodiments, a composition comprises at least three fermentates: (i) a first fermentate of a bacterial strain comprising a 16S rDNA sequence with at least 99% sequence identity to the nucleotide sequence of any one of SEQ ID NOs: 1-4; (ii) a second fermentate of a bacterial strain comprising a 16S rDNA sequence with at least 99% sequence identity to the nucleotide sequence of any one of SEQ ID NOs: 5-6; and (iii) a third fermentate of a bacterial strain comprising a 16S rDNA sequence with at least 99% sequence identity to the nucleotide sequence of SEQ ID NO: 9. In some embodiments, a composition comprises at least three fermentates: (i) a first fermentate of a bacterial strain belonging to the species Bacillus subtilis, Bacillus amyloliquefaciens, Bacillus licheniformis, or Bacillus pumilus; (ii) a second fermentate of a bacterial strain belonging to the species Pediococcus pentosaceus or Lactiplantibacillus plantarum; and (iii) a third fermentate of a bacterial strain belonging to the species Bacillus subtilis. The skilled artisan will appreciate that each of the 8 individual combinations formed by one of the 4 options of (i), and one of the 2 options of (ii), with the fermentate of (iii) is contemplated.

Some aspects relate to compositions (and/or nutritional supplements, dental supplements, food products, and/or oral care products comprising a composition) comprising at least two fermentates: (i) a first fermentate of a bacterial strain comprising a 16S rDNA sequence with at least 97% sequence identity to the nucleotide sequence of any one of SEQ ID NOs: 1-4; and (ii) a second fermentate of a bacterial strain comprising a 16S rDNA sequence with at least 97% sequence identity to the nucleotide sequence of any one of SEQ ID NOs: 5-8. In some embodiments, a composition comprises at least two fermentates: (i) a first fermentate of a bacterial strain comprising a 16S rDNA sequence with at least 99% sequence identity to the nucleotide sequence of any one of SEQ ID NOs: 1-4; and (ii) a second fermentate of a bacterial strain comprising a 16S rDNA sequence with at least 99% sequence identity to the nucleotide sequence of any one of SEQ ID NOs: 5-8. In some embodiments, a composition comprises at least two fermentates: (i) a first fermentate of a bacterial strain belonging to the species Bacillus subtilis, Bacillus amyloliquefaciens, Bacillus licheniformis, or Bacillus pumilus; and (ii) a second fermentate of a bacterial strain belonging to the species Pediococcus pentosaceus, Lactiplantibacillus plantarum, Lactobacillus delbrueckii, or Limosilactobacillus fermentum. The skilled artisan will appreciate that each of the 16 individual combinations formed by one of the 4 options of (i), and one of the 4 options of (ii) is contemplated.

Some aspects relate to compositions (and/or nutritional supplements, dental supplements, food products, and/or oral care products comprising a composition) comprising (a) a fermentate of a bacterial strain that disrupts a biofilm of an oral microbe; and (b) at least one of (i) a fermentate of a bacterial strain that produces one or more compounds that are able to reduce the abundance of one or more volatile sulfur compounds (VSCs); (ii) a fermentate of a bacterial strain that produces one or more antioxidants; and/or (iii) a fermentate of a bacterial strain that enhances a function of one or more other bacterial strains that are sources of other fermentates in the composition.

In some embodiments, a bacterial strain from which a fermentate is obtained is capable of a(i) disrupting a biofilm of an oral microbe; b(i) producing one or more compounds that are able to reduce the abundance of one or more volatile sulfur compounds (VSCs); b(ii) producing one or more antioxidants; and/or b(iii) enhancing a function of one or more other bacterial strains that are sources of other fermentates of the composition. Exemplary bacterial strains determined to be capable of b(i) producing one or more compounds that are able to reduce the abundance of one or more volatile sulfur compounds (VSCs); b(ii) producing one or more antioxidants; and/or b(iii) enhancing a function of one or more other bacterial strains of a(i) and/or b(i) that are sources of other fermentates are listed below in Table 1.

TABLE 1
Exemplary bacterial strains capable of a(i) disrupting a biofilm of an oral microbe,
b(i) producing compounds that reduce the abundance of VSCs, b(ii) producing
antioxidants, and/or b(iii) enhancing functions of other bacterial strains of a(i) and b(ii).

| | | | Species | | | | |
|---|---|---|---|---|---|---|---|
| | SEQ ID | SEQ ID NO. of | Representative | | | | |
| | NO. of | Genome | Genome(s) | | | | |
| | 16S | (N50 | (GTDB Release | | | | |
| Bacterial strain | rDNA | score) | 220) | a(i) | b(i) | b(ii) | b(iii) |
| Bacillus subtilis | 1 | 10 | GCF_000009045.1 | Yes | Yes | | Yes |
| | | (141949) | GCF_002153395.1 | | | | |
| Bacillus | 2 | 11 | GCF_000196735.1 | Yes | | | |
| amyloliquefaciens | | (930313) | | | | | |
| Bacillus | 3 | 12 | GCF_000011645.1 | Yes | | | |
| licheniformis | | (605102) | | | | | |
| Bacillus pumilus | 4 | 13 | GCF_900186955.1 | Yes | | | |
| | | (1792941) | GCA_001938995.1 | | | | |
| | | | GCF_002744245.1 | | | | |
| | | | GCF_003431975.1 | | | | |
| | | | GCF_009937765.1 | | | | |
| | | | GCF_024498355.1 | | | | |
| Pediococcus | 5 | 14 | GCF_001437285.1 | | Yes | Yes | |
| pentosaceus | | (102085) | | | | | |
| Lactiplantibacillus | 6 | 15 | GCF_014131735.1 | | Yes | Yes | Yes |
| plantarum | | (164254) | GCA_000463075.2 | | | | |
| Lactobacillus | 7 | | GCA_004556255.1 | | | Yes | |
| delbrueckii | | | | | | | |
| Limosilactobacillus | 8 | 17 | GCF_013394085.1 | | | Yes | |
| fermentum | | (36270) | | | | | |
| Bacillus subtilis | 9 | 18 | GCF_000009045.1 | Yes | Yes | | Yes |
| | | (2066462) | GCF_002153395.1 | | | | |

In some embodiments, the bacterial strain of a(i) from which a fermentate is obtained disrupts a biofilm of an oral microbe. In some embodiments, the bacterial strain is selected from the group consisting of Bacillus subtilis, Bacillus amyloliquefaciens, Bacillus licheniformis, and Bacillus pumilus. In some embodiments, the bacterial strain comprises a 16S rDNA sequence with at least 97% sequence identity to any one of SEQ ID NOs: 1-4 and 9. In some embodiments, the bacterial strain is Bacillus subtilis. In some embodiments, the bacterial strain comprises a 16S rDNA sequence with at least 97% sequence identity to SEQ ID NO: 1 or 9. In some embodiments, the bacterial strain comprises a 16S rDNA sequence with at least 97% sequence identity to SEQ ID NO: 1. In some embodiments, the bacterial strain comprises a 16S rDNA sequence with at least 97% sequence identity to SEQ ID NO: 9. In some embodiments, the bacterial strain is Bacillus amyloliquefaciens. In some embodiments, the bacterial strain comprises a 16S rDNA sequence with at least 97% sequence identity to SEQ ID NO: 2. In some embodiments, the bacterial strain is Bacillus licheniformis. In some embodiments, the bacterial strain comprises a 16S rDNA sequence with at least 97% sequence identity to SEQ ID NO: 3. In some embodiments, the bacterial strain is Bacillus pumilus. In some embodiments, the bacterial strain comprises a 16S rDNA sequence with at least 97% sequence identity to SEQ ID NO: 4. In some embodiments, the bacterial strain of a(i) comprises a genome with at least 95.0% average nucleotide identity (ANI) and an alignment fraction (AF) of at least 20.0% to a reference genome of any one of Accession Nos. GCF_000009045.1, GCF_002153395.1, GCF_000196735.1, GCF_000011645.1, GCF_900186955.1, GCA_001938995.1, GCF_002744245.1, GCF_003431975.1, GCF_009937765.1, and GCF_024498355.1. In some embodiments, the bacterial strain of a(i) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of Accession Nos. GCF_000009045.1, GCF_002153395.1, GCF_000196735.1, GCF_000011645.1, GCF_900186955.1, GCA_001938995.1, GCF_002744245.1, GCF_003431975.1, GCF_009937765.1, and GCF_024498355.1. In some embodiments, the bacterial strain of a(i) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of Accession Nos. GCF_000009045.1, GCF_002153395.1, GCF_000196735.1, GCF_000011645.1, GCF_900186955.1, GCA_001938995.1, GCF_002744245.1, GCF_003431975.1, GCF_009937765.1, and GCF_024498355.1.

In some embodiments, the bacterial strain of a(i) comprises a genome with at least 95.0% average nucleotide identity (ANI) and an alignment fraction (AF) of at least 20.0% to a reference genome of any one of SEQ ID NOs: 10, 11, 12, 13, and 18. In some embodiments, the bacterial strain of a(i) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of SEQ ID NOs: 10, 11, 12, 13, and 18. In some embodiments, the bacterial strain of a(i) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of SEQ ID NOs: 10, 11, 12, 13, and 18.

In some embodiments, the bacterial strain of b(i) from which a fermentate is obtained is capable of producing one or more compounds that are able to reduce the abundance of one or more VSCs. In some embodiments, the bacterial strain of b(i) is selected from the group consisting of Pediococcus pentosaceus, Bacillus subtilis, and Lactiplantibacillus plantarum. In some embodiments, the bacterial strain comprises a 16S rDNA sequence with at least 97% sequence identity to any one of SEQ ID NOs: 1, 5, 6, and 9. In some embodiments, the bacterial strain is Bacillus subtilis. In some embodiments, the bacterial strain comprises a 16S rDNA sequence with at least 97% sequence identity to SEQ ID NO: 1 or 9. In some embodiments, the bacterial strain comprises a 16S rDNA sequence with at least 97% sequence identity to SEQ ID NO: 1. In some embodiments, the bacterial strain comprises a 16S rDNA sequence with at least 97% sequence identity to SEQ ID NO: 9. In some embodiments, the bacterial strain is Pediococcus pentosaceus. In some embodiments, the bacterial strain comprises a 16S rDNA sequence with at least 97% sequence identity to SEQ ID NO: 5. In some embodiments, the bacterial strain is Lactiplantibacillus plantarum. In some embodiments, the bacterial strain comprises a 16S rDNA sequence with at least 97% sequence identity to SEQ ID NO: 6. In some embodiments, the bacterial strain of b(i) comprises a genome with at least 95.0% average nucleotide identity (ANI) and an alignment fraction (AF) of at least 20.0% to a reference genome of any one of Accession Nos. GCF_000009045.1, GCF_002153395.1, GCF_001437285.1, GCF_014131735.1, and GCA_000463075.2. In some embodiments, the bacterial strain of b(i) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of Accession Nos. GCF_000009045.1, GCF_002153395.1, GCF_001437285.1, GCF_014131735.1, and GCA_000463075.2. In some embodiments, the bacterial strain of b(i) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of Accession Nos. GCF_000009045.1, GCF_002153395.1, GCF_001437285.1, GCF_014131735.1, and GCA_000463075.2.

In some embodiments, the bacterial strain of b(i) comprises a genome with at least 95.0% average nucleotide identity (ANI) and an alignment fraction (AF) of at least 20.0% to a reference genome of any one of SEQ ID NOs: 10, 14, 15, and 18. In some embodiments, the bacterial strain of b(i) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of SEQ ID NOs: 10, 14, 15, and 18. In some embodiments, the bacterial strain of b(i) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of SEQ ID NOs: 10, 14, 15, and 18.

In some embodiments, the bacterial strain of b(ii) from which a fermentate is obtained is capable of producing one or more antioxidants. In some embodiments, the antioxidant is selected from the group consisting of a vitamin, an organic acid, a polyphenol, an indole-containing compound, and a phenol. In some embodiments, the antioxidant is reactive to the ferric ion-reducing antioxidant power assay. See, e.g., Benzie and Strain, Methods Enzymol. 1999. 299:15-27.

In some embodiments, the antioxidant is a vitamin. In some embodiments, the vitamin is selected from vitamin A, vitamin C, vitamin B1, B2, B6, B12, vitamin D3, vitamin E, and vitamin K.

In some embodiments, the antioxidant is an organic acid. In some embodiments, the organic acid is selected from the group consisting of lactic acid, ascorbic acid, acetic acid, acrylic acid, propionic acid, pyruvic acid, and citric acid.

In some embodiments, the antioxidant is a polyphenol. In some embodiments, the polyphenol is selected from the group consisting of green tea polyphenols, tannins, resveratrol, flavonoids, and anthrocyanins.

In some embodiments, the antioxidant is an indole-containing compound. In some embodiments, the indole-containing compound is selected from the group consisting of indoleacrylic acid, indolepropionic acid, indoleacetic acid, indole-3-carbinol, indole-3-acetamine, indolealdehyde, indolelactic acid, indolepyruvate, tryptophol, and indole.

In some embodiments, the antioxidant is a phenol.

In some embodiments, the bacterial strain of b(ii) that is capable of producing one or more antioxidants is selected from the group consisting of Lactiplantibacillus plantarum, Lactobacillus delbrueckii, and Limosilactobacillus fermentum. In some embodiments, the bacterial strain that is capable of producing one or more antioxidants comprises a 16S rDNA sequence with at least 97% sequence identity to any one of SEQ ID NOs: 6-8. In some embodiments, the bacterial strain that is capable of producing one or more antioxidants is selected from the group consisting of Pediococcus pentosaceus, Lactiplantibacillus plantarum, Lactobacillus delbrueckii, and Limosilactobacillus fermentum. In some embodiments, the bacterial strain that is capable of producing one or more antioxidants comprises a 16S rDNA sequence with at least 97% sequence identity to any one of SEQ ID NOs: 5-8. In some embodiments, the bacterial strain is Lactiplantibacillus plantarum. In some embodiments, the bacterial strain comprises a 16S rDNA sequence with at least 97% sequence identity to SEQ ID NO: 6. In some embodiments, the bacterial strain is Lactobacillus delbrueckii. In some embodiments, the bacterial strain comprises a 16S rDNA sequence with at least 97% sequence identity to SEQ ID NO: 7. In some embodiments, the bacterial strain is Limosilactobacillus fermentum. In some embodiments, the bacterial strain comprises a 16S rDNA sequence with at least 97% sequence identity to SEQ ID NO: 8. In some embodiments, the bacterial strain is Pediococcus pentosaceus. In some embodiments, the bacterial strain comprises a 16S rDNA sequence with at least 97% sequence identity to SEQ ID NO: 5. In some embodiments, the bacterial strain of b(ii) comprises a genome with at least 95.0% average nucleotide identity (ANI) and an alignment fraction (AF) of at least 20.0% to a reference genome of any one of Accession Nos. GCF_001437285.1, GCF_014131735.1, GCA_000463075.2, GCA_004556255.1, and GCF_013394085.1. In some embodiments, the bacterial strain of b(ii) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of Accession Nos. GCF_001437285.1, GCF_014131735.1, GCA_000463075.2, GCA_004556255.1, and GCF_013394085.1. In some embodiments, the bacterial strain of b(ii) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of Accession Nos. GCF_001437285.1, GCF_014131735.1, GCA_000463075.2, GCA_004556255.1, and GCF_013394085.1.

In some embodiments, the bacterial strain of b(ii) comprises a genome with at least 95.0% average nucleotide identity (ANI) and an alignment fraction (AF) of at least 20.0% to a reference genome of any one of SEQ ID NOs: 14, 15, and 17. In some embodiments, the bacterial strain of b(ii) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of SEQ ID NOs: 14, 15, and 17. In some embodiments, the bacterial strain of b(ii) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of SEQ ID NOs: 14, 15, and 17.

In some embodiments, the bacterial strain of b(iii) from which a fermentate is obtained is capable of enhancing a function of one or more other bacterial strains that are sources of other fermentates of the composition. In some embodiments, the bacterial strain is selected from Bacillus subtilis and Lactiplantibacillus plantarum. In some embodiments, the bacterial strain comprises a 16S rDNA sequence identity to any one of SEQ ID NOs: 1, 6, or 9. In some embodiments, the bacterial strain of b(iii) comprises a genome with at least 95.0% average nucleotide identity (ANI) and an alignment fraction (AF) of at least 20.0% to a reference genome of any one of Accession Nos. GCF_000009045.1, GCF_002153395.1, GCF_014131735.1, and GCA_000463075.2. In some embodiments, the bacterial strain of b(iii) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of Accession Nos. GCF_000009045.1, GCF_002153395.1, GCF_014131735.1, and GCA_000463075.2. In some embodiments, the bacterial strain of b(iii) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of Accession Nos. GCF_000009045.1, GCF_002153395.1, GCF_014131735.1, and GCA_000463075.2.

In some embodiments, the bacterial strain of b(iii) comprises a genome with at least 95.0% average nucleotide identity (ANI) and an alignment fraction (AF) of at least 20.0% to a reference genome of any one of SEQ ID NOs: 10, 15, and 18. In some embodiments, the bacterial strain of b(iii) comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to a reference genome of any one of SEQ ID NOs: 10 and 15. In some embodiments, the bacterial strain of b(iii) comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to a reference genome of any one of SEQ ID NOs: 10, 15 and 18.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1. In some embodiments, the bacterial strain comprising a 16S rDNA sequence with at least 97% sequence identity to SEQ ID NO: 1 belongs to the species Bacillus subtilis. In some embodiments, the bacterial strain comprising a 16S rDNA sequence with at least 97% sequence identity to SEQ ID NO: 10 comprises a genome with at least 95.0% average nucleotide identity (ANI) and an alignment fraction (AF) of at least 20.0% to the reference genome of SEQ ID NO: 10. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 10. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 10.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 2. In some embodiments, the bacterial strain comprising a 16S rDNA sequence with at least 97% sequence identity to SEQ ID NO: 2 belongs to the species Bacillus amyloliquefaciens. In some embodiments, the bacterial strain comprising a 16S rDNA sequence with at least 97% sequence identity to SEQ ID NO: 11 comprises a genome with at least 95.0% average nucleotide identity (ANI) and an alignment fraction (AF) of at least 20.0% to the reference genome of SEQ ID NO: 11. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 11. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 11.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 3. In some embodiments, the bacterial strain comprising a 16S rDNA sequence with at least 97% sequence identity to SEQ ID NO: 3 belongs to the species Bacillus licheniformis. In some embodiments, the bacterial strain comprising a 16S rDNA sequence with at least 97% sequence identity to SEQ ID NO: 12 comprises a genome with at least 95.0% average nucleotide identity (ANI) and an alignment fraction (AF) of at least 20.0% to the reference genome of SEQ ID NO: 12. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 12. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 12.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 4. In some embodiments, the bacterial strain comprising a 16S rDNA sequence with at least 97% sequence identity to SEQ ID NO: 4 belongs to the species Bacillus pumilus. In some embodiments, the bacterial strain comprising a 16S rDNA sequence with at least 97% sequence identity to SEQ ID NO: 13 comprises a genome with at least 95.0% average nucleotide identity (ANI) and an alignment fraction (AF) of at least 20.0% to the reference genome of SEQ ID NO: 13. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 13. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 13.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 5. In some embodiments, the bacterial strain comprising a 16S rDNA sequence with at least 97% sequence identity to SEQ ID NO: 5 belongs to the species Pediococcus pentosaceus. In some embodiments, the bacterial strain comprising a 16S rDNA sequence with at least 97% sequence identity to SEQ ID NO: 14 comprises a genome with at least 95.0% average nucleotide identity (ANI) and an alignment fraction (AF) of at least 20.0% to the reference genome of SEQ ID NO: 14. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 14. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 14.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 6. In some embodiments, the bacterial strain comprising a 16S rDNA sequence with at least 97% sequence identity to SEQ ID NO: 6 belongs to the species Lactiplantibacillus plantarum. In some embodiments, the bacterial strain comprising a 16S rDNA sequence with at least 97% sequence identity to SEQ ID NO: 15 comprises a genome with at least 95.0% average nucleotide identity (ANI) and an alignment fraction (AF) of at least 20.0% to the reference genome of SEQ ID NO: 15. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 15. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 15.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 7. In some embodiments, the bacterial strain comprising a 16S rDNA sequence with at least 97% sequence identity to SEQ ID NO: 7 belongs to the species Lactobacillus delbrueckii.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 8. In some embodiments, the bacterial strain comprising a 16S rDNA sequence with at least 97% sequence identity to SEQ ID NO: 8 belongs to the species Limosilactobacillus fermentum. In some embodiments, the bacterial strain comprising a 16S rDNA sequence with at least 97% sequence identity to SEQ ID NO: 17 comprises a genome with at least 95.0% average nucleotide identity (ANI) and an alignment fraction (AF) of at least 20.0% to the reference genome of SEQ ID NO: 17. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 17. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 17.

In some embodiments, a bacterial strain, e.g., that produces a fermentate, has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 9. In some embodiments, the bacterial strain comprising a 16S rDNA sequence with at least 97% sequence identity to SEQ ID NO: 9 belongs to the species Bacillus subtilis. In some embodiments, the bacterial strain comprising a 16S rDNA sequence with at least 97% sequence identity to SEQ ID NO: 18 comprises a genome with at least 95.0% average nucleotide identity (ANI) and an alignment fraction (AF) of at least 20.0% to the reference genome of SEQ ID NO: 18. In some embodiments, the bacterial strain comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 18. In some embodiments, the bacterial strain comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 18.

The bacterial strain comprising a 16S rDNA sequence provided by SEQ ID NO: 1 (also referred to herein as “Strain 1”) has the highest homology with a bacterial strain of the species Bacillus subtilis:

Strain 1 16S ribosomal RNA coding sequence
(16S rDNA)
(SEQ ID NO: 1)
ATGTTAGCGGCGGACGGGTGAGTAACACGTGGGTAACCTGCCTGTAAGAC
TGGGATAACTCCGGGAAACCGGGGCTAATACCGGATGGTTGTTTGAACCG
CATGGITCAAACATAAAAGGTGGCTTCGGCTACCACTTACAGATGGACCC
GCGGCGCATTAGCTAGTTGGTGAGGTAACGGCTCACCAAGGCAACGATGC
GTAGCCGACCTGAGAGGGTGATCGGCCACACTGGGACTGAGACACGGCCC
AGACTCCTACGGGAGGCAGCAGTAGGGAATCTTCCGCAATGGACGAAAGT
CTGACGGAGCAACGCCGCGTGAGTGATGAAGGTTTTCGGATCGTAAAGCT
CTGTTGTTAGGGAAGAACAAGTACCGTTCGAATAGGGCGGTACCTTGACG
GTACCTAACCAGAAAGCCACGGCTAACTACGTGCCAGCAGCCGCGGTAAT
ACGTAGGTGGCAAGCGTTGTCCGGAATTATTGGGCGTAAAGGGCTCGCAG
GCGGTTTCTTAAGTCTGATGTGAAAGCCCCCGGCTCAACCGGGGAGGGTC
ATTGGAAACTGGGGAACTTGAGTGCAGAAGAGGAGAGTGGAATTCCACGT
GTAGCGGTGAAATGCGTAGAGATGTGGAGGAACACCAGTGGCGAAGGCGA
CTCTCTGGTCTGTAACTGACGCTGAGGAGCGAAAGCGTGGGGAGCGAACA
GGATTAGATACCCTGGTAGTCCACGCCGTAAACGATGAGTGCTAAGTGTT
AGGGGGTTTCCGCCCCTTAGTGCTGCAGCTAACGCATTAAGCACTCCGCC
TGGGGAGTACGGTCGCAAGACTGAAACTCAAAGGAATTGACGGGGGCCCG
CACAAGCGGTGGAGCATGTGGTTTAATTCGAAGCAACGCGAAGAACCTTA
CCAGGTCTTGACATCCTCTGACAATCCTAGAGATAGGACGTCCCCTTCGG
GGGCAGAGTGACAGGTGGTGCATGGITGTCGTCAGCTCGTGTCGTGAGAT
GTTGGGTTAAGTCCCGCAACGAGCGCAACCCTTGATCTTAGTTGCCAGCA
TTCAGTTGGGCACTCTAAGGTGACTGCCGGTGACAAACCGGAGGAAGGTG
GGGATGACGTCAAATCATCATGCCCCTTATGACCTGGGCTACACACGTGC
TACAATGGACAGAACAAAGGGCAGCGAAACCGCGAGGTTAAGCCAATCCC
ACAAATCTGTTCTCAGTTCGGATCGCAGTCTGCAACTCGACTGCGTGAAG
CTGGAATCGCTAGTAATCGCGGATCAGCATGCCGCGGTGAATACGTTCCC
GGGCCTTGTACACACCGCCCGTCACACCACGAGAGTTTGTAACACCCGAA
GTCGGTGAGGTAACCTTTTAGGAGCCAGCCGCCGAAGGTGGGACAGATGA
TTGGGGTGAAGTCGTAACAAGGTAGCCGTATCGGAAGGTGCGGCTGGATC
ACCTCCTTT

The bacterial strain comprising a 16S rDNA sequence provided by SEQ ID NO: 2 (also referred to herein as “Strain 2”) has the highest homology with a bacterial strain of the species Bacillus amyloliquefaciens:

Strain 2 16S ribosomal RNA coding sequence
(16S rDNA)
(SEQ ID NO: 2)
CGGAGAGTTTGATCCTGGCTCAGGACGAACGCTGGCGGCGTGCCTAATAC
ATGCAAGTCGAGCGGACAGATGGGAGCTTGCTCCCTGATGTTAGCGGCGG
ACGGGTGAGTAACACGTGGGTAACCTGCCTGTAAGACTGGGATAACTCCG
GGAAACCGGGGCTAATACCGGATGCTTGTTTGAACCGCATGGTTCAAACA
TAAAAGGTGGCTTCGGCTACCACTTACAGATGGACCCGCGGCGCATTAGC
TAGTTGGTGAGGTAACGGCTCACCAAGGCGACGATGCGTAGCCGACCTGA
GAGGGTGATCGGCCACACTGGGACTGAGACACGGCCCAGACTCCTACGGG
AGGCAGCAGTAGGGAATCTTCCGCAATGGACGAAAGTCTGACGGAGCAAC
GCCGCGTGAGTGATGAAGGTTTTCGGATCGTAAAGCTCTGTTGTTAGGGA
AGAACAAGTGCCGTTCAAATAGGGCGGCACCTTGACGGTACCTAACCAGA
AAGCCACGGCTAACTACGTGCCAGCAGCCGCGGTAATACGTAGGTGGCAA
GCGTTGTCCGGAATTATTGGGCGTAAAGGGCTCGCAGGCGGTTTCTTAAG
TCTGATGTGAAAGCCCCCGGCTCAACCGGGGAGGGTCATTGGAAACTGGG
GAACTTGAGTGCAGAAGAGGAGAGTGGAATTCCACGTGTAGCGGTGAAAT
GCGTAGAGATGTGGAGGAACACCAGTGGCGAAGGCGACTCTCTGGTCTGT
AACTGACGCTGAGGAGCGAAAGCGTGGGGAGCGAACAGGATTAGATACCC
TGGTAGTCCACGCCGTAAACGATGAGTGCTAAGTGTTAGGGGGTTTCCGC
CCCTTAGTGCTGCAGCTAACGCATTAAGCACTCCGCCTGGGGAGTACGGT
CGCAAGACTGAAACTCAAAGGAATTGACGGGGGCCCGCACAAGCGGTGGA
GCATGTGGTTTAATTCGAAGCAACGCGAAGAACCTTACCAGGTCTTGACA
TCCTCTGACAATCCTAGAGATAGGACGTCCCCTTCGGGGGCAGAGTGACA
GGTGGTGCATGGTTGTCGTCAGCTCGTGTCGTGAGATGTTGGGTTAAGTC
CCGCAACGAGCGCAACCCTTGATCTTAGTTGCCAGCATTCAGTTGGGCAC
TCTAAGGTGACTGCCGGTGACAAACCGGAGGAAGGTGGGGATGACGTCAA
ATCATCATGCCCCTTATGACCTGGGCTACACACGTGCTACAATGGGCAGA
ACAAAGGGCAGCGAAACCGCGAGGTTAAGCCAATCCCACAAATCTGTTCT
CAGTTCGGATCGCAGTCTGCAACTCGACTGCGTGAAGCTGGAATCGCTAG
TAATCGCGGATCAGCATGCCGCGGTGAATACGTTCCCGGGCCTTGTACAC
ACCGCCCGTCACACCACGAGAGTTTGTAACACCCGAAGTCGGTGAGGTAA
CCTTTTTGGAGCCAGCCGCCGAAGGTGGGACAGATGATTGGGGTGAAGTC
GTAACAAGGTAGCCGTATCGGAAGGTGCGGCTGGATCACCTCCTTT

The bacterial strain comprising a 16S rDNA sequence provided by SEQ ID NO: 3 (also referred to herein as “Strain 3”) has the highest homology with a bacterial strain of the species Bacillus licheniformis:

Strain 3 16S ribosomal RNA coding sequence
(16S rDNA)
(SEQ ID NO: 3)
CACTTACAGATGGACCCGCGGCGCATTAGCTAGTTGGTGAGGTAACGGCT
CACCAAGGCGACGATGCGTAGCCGACCTGAGAGGGTGATCGGCCACACTG
GGACTGAGACACGGCCCAGACTCCTACGGGAGGCAGCAGTAGGGAATCTT
CCGCAATGGACGAAAGTCTGACGGAGCAACGCCGCGTGAGTGATGAAGGT
TTTCGGATCGTAAAACTCTGTTGTTAGGGAAGAACAAGTACCGTTCGAAT
AGGGCGGTACCTTGACGGTACCTAACCAGAAAGCCACGGCTAACTACGTG
CCAGCAGCCGCGGTAATACGTAGGTGGCAAGCGTTGTCCGGAATTATTGG
GCGTAAAGCGCGCGCAGGCGGTTTCTTAAGTCTGATGTGAAAGCCCCCGG
CTCAACCGGGGAGGGTCATTGGAAACTGGGGAACTTGAGTGCAGAAGAGG
AGAGTGGAATTCCACGTGTAGCGGTGAAATGCGTAGAGATGTGGAGGAAC
ACCAGTGGCGAAGGCGACTCTCTGGTCTGTAACTGACGCTGAGGCGCGAA
AGCGTGGGGAGCGAACAGGATTAGATACCCTGGTAGTCCACGCCGTAAAC
GATGAGTGCTAAGTGTTAGAGGGTTTCCGCCCTTTAGTGCTGCAGCAAAC
GCATTAAGCACTCCGCCTGGGGAGTACGGTCGCAAGACTGAAACTCAAAG
GAATTGACGGGGGCCCGCACAAGCGGTGGAGCATGTGGTTTAATTCGAAG
CAACGCGAAGAACCTTACCAGGTCTTGACATCCTCTGACAACCCTAGAGA
TAGGGCTTCCCCTTCGGGGGCAGAGTGACAGGTGGTGCATGGTTGTCGTC
AGCTCGTGTCGTGAGATGTTGGGTTAAGTCCCGCAACGAGCGCAACCCTT
GATCTTAGTTGCCAGCATTCAGTTGGGCACTCTAAGGTGACTGCCGGTGA
CAAACCGGAGGAAGGTGGGGATGACGTCAAATCATCATGCCCCTTATGAC
CTGGGCTACACACGTGCTACAATGGGCAGAACAAAGGGCAGCGAAGCCGC
GAGGCTAAGCCAATCCCACAAATCTGTTCTCAGTTCGGATCGCAGTCTGC
AACTCGACTGCGTGAAGCTGGAATCGCTAGTAATCGCGGATCAGCATGCC
GCGGTGAATACGTTCCCGGGCCTTGTACACACCGCCCGTCACACCACGAG
AGTTTGTAACACCCGAAGTCGGTGAGGTAACCTTTTGGAGCCAGCCGCCG
AAGGTGGGACAGATGATTGGGGTGAAGTCGTAACAAGGTAGCCGTATCGG
AAGGTGCGGCTGGATCACCTCCTTT

The bacterial strain comprising a 16S rDNA sequence provided by SEQ ID NO: 4 (also referred to herein as “Strain 4”) has the highest homology with a bacterial strain of the species Bacillus pumilus:

Strain 4 16S ribosomal RNA coding sequence
(16S rDNA)
(SEQ ID NO: 4)
ACGCTGGCGGCGTGCCTAATACATGCAAGTCGAGCGGACAGAAGGGAGCT
TGCTCCCGGATGTTAGCGGCGGACGGGTGAGTAACACGTGGGTAACCTGC
CTGTAAGACTGGGATAACTCCGGGAAACCGGAGCTAATACCGGATAGTTC
CTTGAACCGCATGGTTCAAGGATGAAAGACGGTTTCGGCTGTCACTTACA
GATGGACCCGCGGCGCATTAGCTAGTTGGTGGGGTAATGGCTCACCAAGG
CGACGATGCGTAGCCGACCTGAGAGGGTGATCGGCCACACTGGGACTGAG
ACACGGCCCAGACTCCTACGGGAGGCAGCAGTAGGGAATCTTCCGCAATG
GACGAAAGTCTGACGGAGCAACGCCGCGTGAGTGATGAAGGTTTTCGGAT
CGTAAAGCTCTGTTGTTAGGGAAGAACAAGTGCGAGAGTAACTGCTCGCA
CCTTGACGGTACCTAACCAGAAAGCCACGGCTAACTACGTGCCAGCAGCC
GCGGTAATACGTAGGTGGCAAGCGTTGTCCGGAATTATTGGGCGTAAAGG
GCTCGCAGGCGGITTCTTAAGTCTGATGTGAAAGCCCCCGGCTCAACCGG
GGAGGGTCATTGGAAACTGGGAAACTTGAGTGCAGAAGAGGAGAGTGGAA
TTCCACGTGTAGCGGTGAAATGCGTAGAGATGTGGAGGAACACCAGTGGC
GAAGGCGACTCTCTGGTCTGTAACTGACGCTGAGGAGCGAAAGCGTGGGG
AGCGAACAGGATTAGATACCCTGGTAGTCCACGCCGTAAACGATGAGTGC
TAAGTGTTAGGGGGTTTCCGCCCCTTAGTGCTGCAGCTAACGCATTAAGC
ACTCCGCCTGGGGAGTACGGTCGCAAGACTGAAACTCAAAGGAATTGACG
GGGGCCCGCACAAGCGGTGGAGCATGTGGTTTAATTCGAAGCAACGCGAA
GAACCTTACCAGGTCTTGACATCCTCTGACAACCCTAGAGATAGGGCTTT
CCCTTCGGGGACAGAGTGACAGGTGGTGCATGGTTGTCGTCAGCTCGTGT
CGTGAGATGTTGGGTTAAGTCCCGCAACGAGCGCAACCCTTGATCTTAGT
TGCCAGCATTTAGTTGGGCACTCTAAGGTGACTGCCGGTGACAAACCGGA
GGAAGGTGGGGATGACGTCAAATCATCATGCCCCTTATGACCTGGGCTAC
ACACGTGCTACAATGGACAGAACAAAGGGCTGCGAGACCGCAAGGTTTAG
CCAATCCCATAAATCTGTTCTCAGTTCGGATCGCAGTCTGCAACTCGACT
GCGTGAAGCTGGAATCGCTAGTAATCGCGGATCAGCATGCCGCGGTGAAT
ACGTTCCCGGGCCTTGTACACACCGCCCGTCACACCACGAGAGTTTGCAA
CACCCGAAGTCGGTGAGGTAACCTTTATGGAGCCAGCCGCCGAAGGTGGG
GCAGATGATTGGGGTGAAGTCGTAACAAGGTAGCCGTATCGGAAGGTGCG
GCTGGATCACCTCCTTT

The bacterial strain comprising a 16S rDNA sequence provided by SEQ ID NO: 5 (also referred to herein as “Strain 5”) has the highest homology with a bacterial strain of the species Pediococcus pentosaceus:

Strain 5 16S ribosomal RNA coding sequence
(16S rDNA)
(SEQ ID NO: 5)
ATGAGAGTTTGATCTTGGCTCAGGATGAACGCTGGCGGCGTGCCTAATAC
ATGCAAGTCGAACGAACTTCCGTTAATTGATTATGACGTACTTGTACTGA
TTGAGATTTTAACACGAAGTGAGTGGCGAACGGGTGAGTAACACGTGGGT
AACCTGCCCAGAAGTAGGGGATAACACCTGGAAACAGATGCTAATACCGT
ATAACAGAGAAAACCGCATGGTTTTCTTTTAAAAGATGGCTCTGCTATCA
CTTCTGGATGGACCCGCGGCGTATTAGCTAGTTGGTGAGGTAAAGGCTCA
CCAAGGCAGTGATACGTAGCCGACCTGAGAGGGTAATCGGCCACATTGGG
ACTGAGACACGGCCCAGACTCCTACGGGAGGCAGCAGTAGGGAATCTTCC
ACAATGGACGCAAGTCTGATGGAGCAACGCCGCGTGAGTGAAGAAGGGTT
TCGGCTCGTAAAGCTCTGTTGTTAAAGAAGAACGTGGGTAAGAGTAACTG
TTTACCCAGTGACGGTATTTAACCAGAAAGCCACGGCTAACTACGTGCCA
GCAGCCGCGGTAATACGTAGGTGGCAAGCGTTATCCGGATTTATTGGGCG
TAAAGCGAGCGCAGGCGGTCTTTTAAGTCTAATGTGAAAGCCTTCGGCTC
AACCGAAGAAGTGCATTGGAAACTGGGAGACTTGAGTGCAGAAGAGGACA
GTGGAACTCCATGTGTAGCGGTGAAATGCGTAGATATATGGAAGAACACC
AGTGGCGAAGGCGGCTGTCTGGTCTGCAACTGACGCTGAGGCTCGAAAGC
ATGGGTAGCGAACAGGATTAGATACCCTGGTAGTCCATGCCGTAAACGAT
GATTACTAAGTGTTGGAGGGTTTCCGCCCTTCAGTGCTGCAGCTAACGCA
TTAAGTAATCCGCCTGGGGAGTACGACCGCAAGGTTGAAACTCAAAAGAA
TTGACGGGGGCCCGCACAAGCGGTGGAGCATGTGGTTTAATTCGAAGCTA
CGCGAAGAACCTTACCAGGTCTTGACATCTTCTGACAGTCTAAGAGATTA
GAGGTTCCCTTCGGGGACAGAATGACAGGTGGTGCATGGTTGTCGTCAGC
TCGTGTCGTGAGATGTTGGGTTAAGTCCCGCAACGAGCGCAACCCTTATT
ACTAGTTGCCAGCATTAAGTTGGGCACTCTAGTGAGACTGCCGGTGACAA
ACCGGAGGAAGGTGGGGACGACGTCAAATCATCATGCCCCTTATGACCTG
GGCTACACACGTGCTACAATGGATGGTACAACGAGTCGCGAGACCGCGAG
GTTAAGCTAATCTCTTAAAACCATTCTCAGTTCGGACTGTAGGCTGCAAC
TCGCCTACACGAAGTCGGAATCGCTAGTAATCGCGGATCAGCATGCCGCG
GTGAATACGTTCCCGGGCCTTGTACACACCGCCCGTCACACCATGAGAGT
TTGTAACACCCAAAGCCGGTGGGGTAACCTTTTAGGAGCTAGCCGTCTAA
GGTGGGACAGATGATTAGGGTGAAGTCGTAACAAGGTAGCCGTAGGAGAA
CCTGCGGCTGGATCACCTCCTT

The bacterial strain comprising a 16S rDNA sequence provided by SEQ ID NO: 6 (also referred to herein as “Strain 6”) has the highest homology with a bacterial strain of the species Lactiplantibacillus plantarum:

Strain 6 16S ribosomal RNA coding sequence
(16S rDNA)
(SEQ ID NO: 6)
CTATCACTTTTGGATGGTCCCGCGGCGTATTAGCTAGATGGTGGGGTAAC
GGCTCACCATGGCAATGATACGTAGCCGACCTGAGAGGGTAATCGGCCAC
ATTGGGACTGAGACACGGCCCAAACTCCTACGGGAGGCAGCAGTAGGGAA
TCTTCCACAATGGACGAAAGTCTGATGGAGCAACGCCGCGTGAGTGAAGA
AGGGTTTCGGCTCGTAAAACTCTGTTGTTAAAGAAGAACATATCTGAGAG
TAACTGTTCAGGTATTGACGGTATTTAACCAGAAAGCCACGGCTAACTAC
GTGCCAGCAGCCGCGGTAATACGTAGGTGGCAAGCGTTGTCCGGATTTAT
TGGGCGTAAAGCGAGCGCAGGCGGTTTTTTAAGTCTGATGTGAAAGCCTT
CGGCTCAACCGAAGAAGTGCATCGGAAACTGGGAAACTTGAGTGCAGAAG
AGGACAGTGGAACTCCATGTGTAGCGGTGAAATGCGTAGATATATGGAAG
AACACCAGTGGCGAAGGCGGCTGTCTGGTCTGTAACTGACGCTGAGGCTC
GAAAGTATGGGTAGCAAACAGGATTAGATACCCTGGTAGTCCATACCGTA
AACGATGAATGCTAAGTGTTGGAGGGTTTCCGCCCTTCAGTGCTGCAGCT
AACGCATTAAGCATTCCGCCTGGGGAGTACGGCCGCAAGGCTGAAACTCA
AAGGAATTGACGGGGGCCCGCACAAGCGGTGGAGCATGTGGTTTAATTCG
AAGCTACGCGAAGAACCTTACCAGGTCTTGACATACTATGCAAATCTAAG
AGATTAGACGTTCCCTTCGGGGACATGGATACAGGTGGTGCATGGTTGTC
GTCAGCTCGTGTCGTGAGATGTTGGGTTAAGTCCCGCAACGAGCGCAACC
CTTATTATCAGTTGCCAGCATTAAGTTGGGCACTCTGGTGAGACTGCCGG
TGACAAACCGGAGGAAGGTGGGGATGACGTCAAATCATCATGCCCCTTAT
GACCTGGGCTACACACGTGCTACAATGGATGGTACAACGAGTTGCGAACT
CGCGAGAGTAAGCTAATCTCTTAAAGCCATTCTCAGTTCGGATTGTAGGC
TGCAACTCGCCTACATGAAGTCGGAATCGCTAGTAATCGCGGATCAGCAT
GCCGCGGTGAATACGTTCCCGGGCCTTGTACACACCGCCCGTCACACCAT
GAGAGTTTGTAACACCCAAAGTCGGTGGGGTAACCTTTTAGGAACCAGCC
GCCTAAGGTGGGACAGATGATTAGGGTGAAGTCGTAACAAGGTAGCCGTA
GGAGAACCTGCGGCTGGATCACCTCCTT

The bacterial strain comprising a 16S rDNA sequence provided by SEQ ID NO: 7 (also referred to herein as “Strain 7”) has the highest homology with a bacterial strain of the species Lactobacillus delbrueckii:

Strain 7 16S ribosomal RNA coding sequence
(16S rDNA)
(SEQ ID NO: 7)
AGAGTTTGATCCTGGCTCAGGACGAACGCTGGCGGCGTGCCTAATACATG
CAAGTCGAGCGAGCTGAATTCAAAGATCCCTTCGGGGTGATTTGTTGGAC
GCTAGCGGCGGATGGGTGAGTAACACGTGGGCAATCTGCCCTAAAGACTG
GGATACCACTTGGAAACAGGTGCTAATACCGGATAACAACATGAATCGCA
TGATTCAAGTTTGAAAGGCGGCGCAAGCTGTCACTTTAGGATGAGCCCGC
GGCGCATTAGCTAGTTGGTGGGGTAAAGGCCTACCAAGGCAATGATGCGT
AGCCGAGTTGAGAGACTGATCGGCCACATTGGGACTGAGACACGGCCCAA
ACTCCTACGGGAGGCAGCAGTAGGGAATCTTCCACAATGGACGCAAGTCT
GATGGAGCAACGCCGCGTGAGTGAAGAAGGTCTTCGGATCGTAAAGCTCT
GTTGTTGGTGAAGAAGGATAGAGGCAGTAACTGGTCTTTATTTGACGGTA
ATCAACCAGAAAGTCACGGCTAACTACGTGCCAGCAGCCGCGGTAATACG
TAGGTGGCAAGCGTTGTCCGGATTTATTGGGCGTAAAGCGAGCGCAGGCG
GAATGATAAGTCTGATGTGAAAGCCCACGGCTCAACCGTGGAACTGCATC
GGAAACTGTCATTCTTGAGTGCAGAAGAGGAGAGTGGAACTCCATGTGTA
GCGGTGGAATGCGTAGATATATGGAAGAACACCAGTGGCGAAGGCGGCTC
TCTGGTCTGCAACTGACGCTGAGGCTCGAAAGCATGGGTAGCGAACAGGA
TTAGATACCCTGGTAGTCCATGCCGTAAACGATGAGCGCTAGGTGTTGGG
GACTTTCCNGTCCTCAGTGCCGCAGCAAACGCATTAAGCGCTCCGCCTGG
GGAGTACGACCGCAAGGTTGAAACTCAAAGGAATTGACGGGGGCCCGCAC
AAGCGGTGGAGCATGTGGTTTAATTCGAAGCAACGCGAAGAACCTTACCA
GGTCTTGACATCCTGCGCTACACCTAGAGATAGGTGGTTCCCTTCGGGGA
CGCAGAGACAGGTGGTGCATGGCTGTCGTCAGCTCGTGTCGTGAGATGTT
GGGTTAAGTCCCGCAACGAGCGCAACCCTTGTCTTTAGITGCCATCATTA
AGTTGGGCACTCTAAAGAGACTGCCGGTGACAAACCGGAGGAAGGTGGGG
ATGACGTCAAGTCATCATGCCCCTTATGACCTGGGCTACACACGTGCTAC
AATGGGCAGTACAACGAGAAGCNAACCCGCGAGGGTAAGCGGATCTCTTA
AAGCTGTTCTCAGTTCGGACTGCAGGCTGCAACTCGCCTGCACGAAGCTG
GAATCGCTAGTAATCGCGGATCAGCACGCCGCGGTGAATACGTTCCCGGG
CCTTGTACACACCGCCCGTCACACCATGGAAGTCTGCAATGCCCAAAGTC
GGTGAGATAACCTTTATAGGAGTCAGCCGCCTAAGGCAGGGCAGATGACT
GGGGTGAAGTCGTAACAAGGTAACC

The bacterial strain comprising a 16S rDNA sequence provided by SEQ ID NO: 8 (also referred to herein as “Strain 8”) has the highest homology with a bacterial strain of the species Limosilactobacillus fermentum:

Strain 8 16S ribosomal RNA coding sequence
(16S rDNA)
(SEQ ID NO: 8)
CGGCGGTGTGCCTAATACATGCAAGTCGAACGCGTTGGCCCAATTGATTG
ATGGTGCTTGCACCTGATTGATTTTGGTCGCCAACGAGTGGCGGACGGGT
GAGTAACACGTAGGTAACCTGCCCAGAAGCGGGGGACAACATTTGGAAAC
AGATGCTAATACCGCATAACAACGTTGTTCGCATGAACAACGCTTAAAAG
ATGGCTTCTCGCTATCACTTCTGGATGGACCTGCGGTGCATTAGCTTGTT
GGTGGGGTAACGGCCTACCAAGGCGATGATGCATAGCCGAGTTGAGAGAC
TGATCGGCCACAATGGGACTGAGACACGGCCCATACTCCTACGGGAGGCA
GCAGTAGGGAATCTTCCACAATGGGCGCAAGCCTGATGGAGCAACACCGC
GTGAGTGAAGAAGGGTTTCGGCTCGTAAAGCTCTGTTGTTAAAGAAGAAC
ACGTATGAGAGTAACTGTTCATACGTTGACGGTATTTAACCAGAAAGTCA
CGGCTAACTACGTGCCAGCAGCCGCGGTAATACGTAGGTGGCAAGCGTTA
TCCGGATTTATTGGGCGTAAAGAGAGTGCAGGCGGTTTTCTAAGTCTGAT
GTGAAAGCCTTCGGCTTAACCGGAGAAGTGCATCGGAAACTGGATAACTT
GAGTGCAGAAGAGGGTAGTGGAACTCCATGTGTAGCGGTGGAATGCGTAG
ATATATGGAAGAACACCAGTGGCGAAGGCGGCTACCTGGTCTGCAACTGA
CGCTGAGACTCGAAAGCATGGGTAGCGAACAGGATTAGATACCCTGGTAG
TCCATGCCGTAAACGATGAGTGCTAGGTGTTGGAGGGTTTCCGCCCTTCA
GTGCCGGAGCTAACGCATTAAGCACTCCGCCTGGGGAGTACGACCGCAAG
GTTGAAACTCAAAGGAATTGACGGGGGCCCGCACAAGCGGTGGAGCATGT
GGTTTAATTCGAAGCTACGCGAAGAACCTTACCAGGTCTTGACATCTTGC
GCCAACCCTAGAGATAGGGCGTTTCCTTCGGGAACGCAATGACAGGTGGT
GCATGGTCGTCGTCAGCTCGTGTCGTGAGATGTTGGGTTAAGTCCCGCAA
CGAGCGCAACCCTTGTTACTAGTTGCCAGCATTAAGTTGGGCACTCTAGT
GAGACTGCCGGTGACAAACCGGAGGAAGGTGGGGACGACGTCAGATCATC
ATGCCCCTTATGACCTGGGCTACACACGTGCTACAATGGACGGTACAACG
AGTCGCGAACTCGCGAGGGCAAGCAAATCTCTTAAAACCGTTCTCAGTTC
GGACTGCAGGCTGCAACTCGCCTGCACGAAGTCGGAATCGCTAGTAATCG
CGGATCAGCATGCCGCGGTGAATACGTTCCCGGGCCTTGTACACACCGCC
CGTCACACCATGAGAGTTTGTAACACCCAAAGTCGGTGGGGTAACCTTTT
AGGAGCCAGCCGCCTAAGGTGGGACAGATGATTAGGGTGAAGTCGTAACA
AG

The bacterial strain comprising a 16S rDNA sequence provided by SEQ ID NO: 9 (also referred to herein as “Strain 9”) has the highest homology with a bacterial strain of the species Bacillus subtilis:

Strain 9 16S ribosomal RNA coding sequence
(16S rDNA)
(SEQ ID NO: 9)
GTTAGCGGCGGACGGGTGAGTAACACGTGGGTAACCTGCCTGTAAGACTG
GGATAACTCCGGGAAACCGGGGCTAATACCGGATGGTTGTTTGAACCGCA
TGGTTCAAACATAAAAGGTGGCTTCGGCTACCACTTACAGATGGACCCGC
GGCGCATTAGCTAGTTGGTGAGGTAACGGCTCACCAAGGCGACGATGCGT
AGCCGACCTGAGAGGGTGATCGGCCACACTGGGACTGAGACACGGCCCAG
ACTCCTACGGGAGGCAGCAGTAGGGAATCTTCCGCAATGGACGAAAGTCT
GACGGAGCAACGCCGCGTGAGTGATGAAGGTTTTCGGATCGTAAAGCTCT
GTTGTTAGGGAAGAACAAGTACCGTTCGAATAGGGCGGTACCTTGACGGT
ACCTAACCAGAAAGCCACGGCTAACTACGTGCCAGCAGCCGCGGTAATAC
GTAGGTGGCAAGCGTTGTCCGGAATTATTGGGCGTAAAGGGCTCGCAGGC
GGTTTCTTAAGTCTGATGTGAAAGCCCCCGGCTCAACCGGGGAGGGTCAT
TGGAAACTGGGGAACTTGAGTGCAGAAGAGGAGAGTGGAATTCCACGTGT
AGCGGTGAAATGCGTAGAGATGTGGAGGAACACCAGTGGCGAAGGCGACT
CTCTGGTCTGTAACTGACGCTGAGGAGCGAAAGCGTGGGGAGCGAACAGG
ATTAGATACCCTGGTAGTCCACGCCGTAAACGATGAGTGCTAAGTGTTAG
GGGGTTTCCGCCCCTTAGTGCTGCAGCTAACGCATTAAGCACTCCGCCTG
GGGAGTACGGTCGCAAGACTGAAACTCAAAGGAATTGACGGGGGCCCGCA
CAAGCGGTGGAGCATGTGGTTTAATTCGAAGCAACGCGAAGAACCTTACC
AGGTCTTGACATCCTCTGACAATCCTAGAGATAGGACGTCCCCTTCGGGG
GCAGAGTGACAGGTGGTGCATGGTTGTCGTCAGCTCGTGTCGTGAGATGT
TGGGTTAAGTCCCGCAACGAGCGCAACCCTTGATCTTAGTTGCCAGCATT
CAGTTGGGCACTCTAAGGTGACTGCCGGTGACAAACCGGAGGAAGGTGGG
GATGACGTCAAATCATCATGCCCCTTATGACCTGGGCTACACACGTGCTA
CAATGGACAGAACAAAGGGCAGCGAAACCGCGAGGTTAAGCCAATCCCAC
AAATCTGTTCTCAGTTCGGATCGCAGTCTGCAACTCGACTGCGTGAAGCT
GGAATCGCTAGTAATCGCGGATCAGCATGCCGCGGTGAATACGTTCCCGG
GCCTTGTACACACCGCCCGTCACACCACGAGAGTTTGTAACACCCGAAGT
CGGTGAGGTAACCTTTTAGGAGCCAGCCGCCGAAGGTGGGACAGATGATT
GGGGTGAAGTCGTAACAAGGTAGCCGTATCGGAAGGTGCGGCTGGATCAC
CTCCTTT

In some embodiments, a composition comprises fermentates of two or more bacterial strains belonging to the species Bacillus subtilis, wherein the bacterial strains belonging to the species Bacillus subtilis are different bacterial strains. In some embodiments, the bacterial mixture comprises one bacterial strain belonging to Bacillus subtilis. In some embodiments, the bacterial mixture comprises two bacterial strains belonging to Bacillus subtilis. In some embodiments, the bacterial mixture comprises 2, 3, 4, 5, 6, 7, 8, 9, or 10 bacterial strains belonging to Bacillus subtilis. In some embodiments, a first bacterial strain belonging to Bacillus subtilis comprises a 16S rDNA sequence with at least 97% sequence identity to the nucleic acid sequence of SEQ ID NO: 1, and a second bacterial strain belonging to Bacillus subtilis comprises a 16S rDNA sequence with at least 97% sequence identity to the nucleic acid sequence of SEQ ID NO: 9, and the 16S rDNA sequences of the first and second bacterial strains belonging to Bacillus subtilis are not 100% identical. In some embodiments, the 16S rDNA sequence of the first bacterial strain belonging to Bacillus subtilis has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Bacillus subtilis.

In some embodiments, a composition comprises fermentates of two or more bacterial strains belonging to the species Bacillus amyloliquefaciens, wherein the bacterial strains belonging to the species Bacillus amyloliquefaciens are different bacterial strains. In some embodiments, the bacterial mixture comprises one bacterial strain belonging to Bacillus amyloliquefaciens. In some embodiments, the bacterial mixture comprises two bacterial strains belonging to Bacillus amyloliquefaciens. In some embodiments, the bacterial mixture comprises 2, 3, 4, 5, 6, 7, 8, 9, or 10 bacterial strains belonging to Bacillus amyloliquefaciens. In some embodiments, the 16S rDNA sequence of the first bacterial strain belonging to Bacillus amyloliquefaciens has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Bacillus amyloliquefaciens.

In some embodiments, a composition comprises fermentates of two or more bacterial strains belonging to the species Bacillus licheniformis, wherein the bacterial strains belonging to the species Bacillus licheniformis are different bacterial strains. In some embodiments, the bacterial mixture comprises a fermentate from one bacterial strain belonging to Bacillus licheniformis. In some embodiments, the bacterial mixture comprises fermentates from two bacterial strains belonging to Bacillus licheniformis. In some embodiments, the composition comprises fermentates from 2, 3, 4, 5, 6, 7, 8, 9, or 10 bacterial strains belonging to Bacillus licheniformis. In some embodiments, the 16S rDNA sequence of the first bacterial strain belonging to Bacillus licheniformis has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Bacillus licheniformis.

In some embodiments, a composition comprises fermentates of two or more bacterial strains belonging to the species Bacillus pumilus, wherein the bacterial strains belonging to the species Bacillus pumilus are different bacterial strains. In some embodiments, the composition comprises a fermentate from one bacterial strain belonging to Bacillus pumilus. In some embodiments, the bacterial mixture comprises fermentates from two bacterial strains belonging to Bacillus pumilus. In some embodiments, the composition comprises fermentates from 2, 3, 4, 5, 6, 7, 8, 9, or 10 bacterial strains belonging to Bacillus pumilus. In some embodiments, the 16S rDNA sequence of the first bacterial strain belonging to Bacillus pumilus has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Bacillus pumilus.

In some embodiments, a composition comprises fermentates of two or more bacterial strains belonging to the species Pediococcus pentosaceus, wherein the bacterial strains belonging to the species Pediococcus pentosaceus are different bacterial strains. In some embodiments, the composition comprises a fermentate from one bacterial strain belonging to Pediococcus pentosaceus. In some embodiments, the composition comprises fermentates from two bacterial strains belonging to Pediococcus pentosaceus. In some embodiments, the composition comprises fermentates from 2, 3, 4, 5, 6, 7, 8, 9, or 10 bacterial strains belonging to Pediococcus pentosaceus. In some embodiments, the 16S rDNA sequence of the first bacterial strain belonging to Pediococcus pentosaceus has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Pediococcus pentosaceus.

In some embodiments, a composition comprises fermentates of two or more bacterial strains belonging to the species Lactiplantibacillus plantarum, wherein the bacterial strains belonging to the species Lactiplantibacillus plantarum are different bacterial strains. In some embodiments, the composition comprises a fermentate from one bacterial strain belonging to Lactiplantibacillus plantarum. In some embodiments, the composition comprises fermentates from two bacterial strains belonging to Lactiplantibacillus plantarum. In some embodiments, the composition comprises fermentates from 2, 3, 4, 5, 6, 7, 8, 9, or 10 bacterial strains belonging to Lactiplantibacillus plantarum. In some embodiments, the 16S rDNA sequence of the first bacterial strain belonging to Lactiplantibacillus plantarum has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Lactiplantibacillus plantarum.

In some embodiments, a composition comprises fermentates of two or more bacterial strains belonging to the species Lactobacillus delbrueckii, wherein the bacterial strains belonging to the species Lactobacillus delbrueckii are different bacterial strains. In some embodiments, the composition comprises a fermentate from one bacterial strain belonging to Lactobacillus delbrueckii. In some embodiments, the composition comprises fermentates from two bacterial strains belonging to Lactobacillus delbrueckii. In some embodiments, the composition comprises fermentates from 2, 3, 4, 5, 6, 7, 8, 9, or 10 bacterial strains belonging to Lactobacillus delbrueckii. In some embodiments, the 16S rDNA sequence of the first bacterial strain belonging to Lactobacillus delbrueckii has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Lactobacillus delbrueckii.

In some embodiments, a composition comprises fermentates of two or more bacterial strains belonging to the species Limosilactobacillus fermentum, wherein the bacterial strains belonging to the species Limosilactobacillus fermentum are different bacterial strains. In some embodiments, the composition comprises a fermentate from one bacterial strain belonging to Limosilactobacillus fermentum. In some embodiments, the composition comprises fermentates from two bacterial strains belonging to Limosilactobacillus fermentum. In some embodiments, the composition comprises fermentates from 2, 3, 4, 5, 6, 7, 8, 9, or 10 bacterial strains belonging to Limosilactobacillus fermentum. In some embodiments, the 16S rDNA sequence of the first bacterial strain belonging to Limosilactobacillus fermentum has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the second bacterial strain belonging to Limosilactobacillus fermentum.

In some embodiments, a composition comprises a fermentate of one or more additional bacterial strains. Additional bacterial strains that produce fermentates may be taxonomically or phylogenetically related to bacterial strains of Bacillus subtilis, Bacillus amyloliquefaciens, Bacillus licheniformis, Bacillus pumilus, Pediococcus pentosaceus, Lactiplantibacillus plantarum, Lactobacillus delbrueckii, and Limosilactobacillus fermentum. In some embodiments, one or more additional bacterial strains belong to the species Bacillus subtilis. In some embodiments, one or more additional bacterial strains belong to the species Bacillus amyloliquefaciens. In some embodiments, one or more additional bacterial strains belong to the species Bacillus licheniformis. In some embodiments, one or more additional bacterial strains belong to the species Bacillus pumilus. In some embodiments, one or more additional bacterial strains belong to the species Pediococcus pentosaceus. In some embodiments, one or more additional bacterial strains belong to the species Lactiplantibacillus plantarum. In some embodiments, one or more additional bacterial strains belong to the species Lactobacillus delbrueckii. In some embodiments, one or more additional bacterial strains belong to the species Limosilactobacillus fermentum.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Bacillus subtilis. In some embodiments, the 16S rDNA sequence of an additional bacterial strain belonging to Bacillus subtilis is not identical to SEQ ID NO: 1 or SEQ ID NO: 9. In some embodiments, an additional bacterial strain belonging to Bacillus subtilis comprises a 16S rDNA sequence that has at least 97%, but less than 100%, sequence identity to the nucleic acid sequence of SEQ ID NO: 1, and less than 100% sequence identity to the nucleic acid sequence of SEQ ID NO: 9. In some embodiments, each additional bacterial strain belonging to the species Bacillus subtilis comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 10 or 18. In some embodiments, each additional bacterial strain belonging to the species Bacillus subtilis comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 10 or 18.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Bacillus amyloliquefaciens. In some embodiments, the 16S rDNA sequence of an additional bacterial strain belonging to Bacillus amyloliquefaciens is not identical to SEQ ID NO: 2. In some embodiments, an additional bacterial strain belonging to Bacillus amyloliquefaciens comprises a 16S rDNA sequence that has at least 97%, but less than 100%, sequence identity to the nucleic acid sequence of SEQ ID NO: 2. In some embodiments, each additional bacterial strain belonging to the species Bacillus amyloliquefaciens comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 11. In some embodiments, each additional bacterial strain belonging to the species Bacillus amyloliquefaciens comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 11.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Bacillus licheniformis. In some embodiments, the 16S rDNA sequence of an additional bacterial strain belonging to Bacillus licheniformis is not identical to SEQ ID NO: 3. In some embodiments, an additional bacterial strain belonging to Bacillus licheniformis comprises a 16S rDNA sequence that has at least 97%, but less than 100%, sequence identity to the nucleic acid sequence of SEQ ID NO: 3. In some embodiments, each additional bacterial strain belonging to the species Bacillus licheniformis comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 12. In some embodiments, each additional bacterial strain belonging to the species Bacillus licheniformis comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 12.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Bacillus pumilus. In some embodiments, the 16S rDNA sequence of an additional bacterial strain belonging to Bacillus pumilus is not identical to SEQ ID NO: 4. In some embodiments, an additional bacterial strain belonging to Bacillus pumilus comprises a 16S rDNA sequence that has at least 97%, but less than 100%, sequence identity to the nucleic acid sequence of SEQ ID NO: 4. In some embodiments, each additional bacterial strain belonging to the species Bacillus pumilus comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 13. In some embodiments, each additional bacterial strain belonging to the species Bacillus pumilus comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 13.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Pediococcus pentosaceus. In some embodiments, the 16S rDNA sequence of an additional bacterial strain belonging to Pediococcus pentosaceus is not identical to SEQ ID NO: 5. In some embodiments, an additional bacterial strain belonging to Pediococcus pentosaceus comprises a 16S rDNA sequence that has at least 97%, but less than 100%, sequence identity to the nucleic acid sequence of SEQ ID NO: 5. In some embodiments, each additional bacterial strain belonging to the species Pediococcus pentosaceus comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 14. In some embodiments, each additional bacterial strain belonging to the species Pediococcus pentosaceus comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 14.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Lactiplantibacillus plantarum. In some embodiments, the 16S rDNA sequence of an additional bacterial strain belonging to Lactiplantibacillus plantarum is not identical to SEQ ID NO: 6. In some embodiments, an additional bacterial strain belonging to Lactiplantibacillus plantarum comprises a 16S rDNA sequence that has at least 97%, but less than 100%, sequence identity to the nucleic acid sequence of SEQ ID NO: 6. In some embodiments, each additional bacterial strain belonging to the species Lactiplantibacillus plantarum comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 15. In some embodiments, each additional bacterial strain belonging to the species Lactiplantibacillus plantarum comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 15.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Lactobacillus delbrueckii. In some embodiments, the 16S rDNA sequence of an additional bacterial strain belonging to Lactobacillus delbrueckii is not identical to SEQ ID NO: 7. In some embodiments, an additional bacterial strain belonging to Lactobacillus delbrueckii comprises a 16S rDNA sequence that has at least 97%, but less than 100%, sequence identity to the nucleic acid sequence of SEQ ID NO: 7.

In some embodiments, a composition comprises fermentates of 2, 3, 4, 5, 6, 7, 8, 9, 10, 11, 12, 13, 14, 15, 16, 17, 18, 19, or 20 additional bacterial strains belonging to the species Limosilactobacillus fermentum. In some embodiments, the 16S rDNA sequence of an additional bacterial strain belonging to Limosilactobacillus fermentum is not identical to SEQ ID NO: 8. In some embodiments, an additional bacterial strain belonging to Limosilactobacillus fermentum comprises a 16S rDNA sequence that has at least 97%, but less than 100%, sequence identity to the nucleic acid sequence of SEQ ID NO: 8. In some embodiments, each additional bacterial strain belonging to the species Limosilactobacillus fermentum comprises a genome with at least 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% ANI to the reference genome of SEQ ID NO: 17. In some embodiments, each additional bacterial strain belonging to the species Limosilactobacillus fermentum comprises a genome with an AF of at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% to the reference genome of SEQ ID NO: 17.

Some embodiments of the compositions described herein comprise fermentates of two or more bacterial strains. In some embodiments, the composition comprises fermentates of at least 2, at least 3 at least 4, at least 5, at least 6, at least 7, at least 8, at least 9, at least 10, at least 11, at least 12, at least 13, at least 14, at least 15, at least 16, at least 17, at least 18, at least 19, or up to 20 total bacterial strains. In some embodiments, the composition comprises fermentates of 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20.

It will be appreciated that the terms “bacterial strains,” “microbial strains” “microbes,” “bacterial cells” and “microorganisms” are used interchangeably herein.

Bacterial strains identified as described herein were compared with sequences in publicly available nucleic acid databases, such as Basic Local Alignment Search Tool (BLAST) to determine closely related genera and species and were analyzed using taxonomic assignment tools, such as RDP Classifier, which assign bacterial taxonomy to representative sequences.

16S ribosomal DNA (16S rDNA) sequences encoding 16S ribosomal RNA (16S rRNA) are provided below for representative bacterial strains. The closest related identified bacterial species were determined based on whole genome sequence analysis and a combination of analysis of full length 16S rDNA sequences and the sequences of single-copy conserved genes compared to publicly available sequence databases. It should be appreciated that multiple bacterial strains disclosed herein may have the highest homology with the same species. In some embodiments, bacterial strains having the same or highly related 16S rDNA variable region sequences are phenotypically distinct. For example, two strains may differ phenotypically by growth rate, antibiotic resistance, phage resistance, cell wall thickness, motility, competence, spore-forming ability, rates of metabolite (e.g., VSC) degradation, ability to degrade a given metabolite, and/or ability to inhibit production of a given molecule. It should further be appreciated that the bacterial strains disclosed herein that have a 16S rDNA sequence with a nucleic acid sequence selected from the group consisting of SEQ ID NOs: 1-9, are also homologous to other strains based on their whole genome sequence, or subset of their whole genome sequence.

It should further be appreciated that the bacterial species described herein may be identified based on the nucleotide sequence of the full length 16S rDNA. Alternatively or in addition, the bacterial species described herein may be identified based on identification of 16S sequences through whole genome sequencing, and by comparing the sequences with 16S databases, or comparing the whole genome sequence, or a subset of their whole genome sequence to sequence databases.

It should be appreciated that the compositions (and/or nutritional supplements and/or dental supplements, food products, and/or oral care products comprising the same) may include fermentates of multiple strains of a particular species. In some embodiments, the composition includes multiple strains of a particular species that are obtained from independent sources, but the strains have the same or highly related 16S rDNA sequences.

Some embodiments relate to bacterial strains with 16S rDNA sequences that have sequence identity to a nucleic acid sequence of any one of the sequences of the bacterial strains or bacterial species described herein. In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity relative to any of the bacterial strains or bacterial species described herein. “Identity” refers to the degree of sequence relatedness between or among sequences as determined by the number of matching positions between strings of two or more nucleotide sequences. “Percent (%) identity” or “percent (%) sequence identity” as it applies to nucleotide sequences is defined as the percentage of nucleotides in the candidate nucleotide sequence that are identical to the nucleotide sequence of a reference sequence after aligning the sequences and introducing gaps, as necessary, to achieve the maximum score allowed by the alignment algorithm and parameters.

Where an alignment between two sequences is contemplated, the first sequence (e.g., candidate sequence) is aligned to the second sequence (e.g., reference sequence) using the Needleman-Wunsch algorithm for global alignment of the two sequences. Needleman & Wunsch, J Mol Biol. 1970. 48:443-453. Where two nucleotide sequences are aligned, the alignment uses an EDNAFULL substitution scoring matrix, a Gap Open penalty of 10, a Gap Extend penalty of 0.5, and no End Gap penalties. The skilled artisan will appreciate that at the time of filing the instant specification, these parameters are the default parameters of the EMBOSS Needle pairwise comparison tool provided by European Bioinformatics Institute (see ebi.ac.uk). Other suitable alignment programs may be used to obtain a global alignment using these parameters, such as BLAST, or the Needleman-Wunsch algorithm may be implemented in a scripting language (e.g., Python).

The percent sequence identity that a candidate sequence (e.g., as present in a bacterial strain encompassed by a claim) has to a reference sequence (e.g., having a SEQ ID NO: specified herein and recited in a claim) is calculated by (i) aligning the candidate sequence to the reference sequence, (ii) determining the number of matching nucleotides between the aligned candidate and reference sequences, and (iii) dividing the number of matching nucleotides by the length of the reference sequence, including any internal gaps introduced into the reference sequence when the two sequences are aligned.

The skilled artisan will appreciate that to determine whether a candidate nucleotide sequence comprises a nucleotide sequence with a given percentage sequence identity to a reference sequence, the denominator (length of reference sequence plus internal gaps) in calculating percent identity need not include gaps shown extending past the ends of the reference sequence in an alignment. Such gaps are added where a candidate sequence contains additional nucleotides that extend beyond the portions of the candidate sequence that align to the 5′ end or 3′ end of the reference sequence.

Some embodiments relate to bacterial strains comprising a genome (i.e., a chromosome having a nucleotide sequence) with a specified average nucleotide identity (ANI) and/or alignment fraction (AF) to a reference genome. ANI refers to average nucleotide identity of a genome (e.g., as present in a bacterial strain recited in a claim) to a reference genome (e.g., as provided in an Accession No. or SEQ ID NO.), when comparing orthologous regions between the two genomes. The skilled artisan will understand that ANI is not a calculation of percent identity obtained by alignment of two full-length genome sequences. Rather, two genomes are compared to identify regions that are conserved between both genomes, and the average nucleotide identity of all conserved regions is calculated. Konstantinidis & Tiedje, Proc Natl Acad Sci USA. 2005. 102 (7): 2567-2572. Where ANI and/or AF are contemplated, these parameters are calculated using FastANI v1.34 and default configuration. See Jain et al., Nat Commun. 2018. 9 (1): 5114. A genome will be understood to require the following to be suitable for calculation of ANI to a reference genome: completeness as determined by detection of at least 100 single-copy BUSCO genes. See Simão et al., Bioinformatics. 2015. 31 (19): 3210-3212; Manni et al., Mol Biol Evol. 2021. 38 (10): 4647-4654.

As will be appreciated by one of ordinary skill in the art, nomenclature regarding bacterial genus and species names may be reclassified to reflect phylogenetic relationships of microorganisms. See, e.g., Zheng et al., Inter. J. System. And Evol. Microbiol. (2020) 70 (4).

Bacterial strains may be obtained from or derived from any suitable source, such as from a food source or an environmental source. As used herein, the term “derived from” in the context of bacterial strains derived from a particular source refers to obtaining a bacterial strain from the source, which may involve isolating and/or propagating cells of a bacterial strain. In some embodiments, the bacterial strains are further manipulated, such as purified and/or analyzed, prior to use. As will be evident to one of ordinary skill in the art, reference to a bacterial strain or cells of a bacterial strain that is derived from a particular source encompasses progeny cells thereof.

Bacterial strains may be derived from a fermented food or beverage, such as cultured milk and yogurt, natto, cheese, kombucha, wine, beer, cider, miso, kimchi, sauerkraut, fermented sausage, among others. Additionally, bacterial strains may be derived from cultured plants or plant protein isolates, including plants or proteins isolated from cereal grains (e.g., oats), oil seeds (e.g., sunflower seeds), legumes, pulses, beans, broad beans, faba beans, peas, chickpeas, cow peas, pigeon peas, lentils, Bambara beans, vetches, and lupins.

In some embodiments, at least one bacterial strain may be purified. In some embodiments, at least one bacterial strain may be isolated. Any of the bacterial strains described herein may be isolated and/or purified, for example, from a source such as a food source (e.g., a fermented food or beverage product) or an environmental source (e.g., soil).

### Compositions Comprising Fermentates

Aspects of the present disclosure relate to compositions comprising fermentates of bacterial strains. As used herein, the term “fermentate” refers to a product obtained by fermenting a bacterial strain in an environment suitable for fermentation, such as a culture medium (e.g., liquid medium). In some embodiments, the fermentate comprises products produced by the bacterial strain(s) during fermentation. In some embodiments, the fermentate comprises products produced by the bacterial strain(s) during fermentation and components of the culture medium. Mixtures of fermentates of different bacterial strains (e.g., a first fermentate of a first bacterial strain and a second fermentate of a second bacterial strain) may be produced by fermenting different bacterial strains separately and combining the resulting individual fermentates, or by fermenting multiple bacterial strains together.

In some embodiments, the bacteria are propagated or manufactured using liquid fermenters, which can support the rapid growth of bacterial species. The fermenters may be, for example, stirred tank reactors or disposable wave bioreactors. Culture media such as BL media and EG media, or similar versions of these media, can be used to support the growth of the bacterial species. The bacterial fermentate can be purified and concentrated from the fermentation broth by suitable techniques, such as centrifugation and filtration, and can optionally be dried and lyophilized by suitable techniques such as those known in the art.

In some embodiments, a composition comprising a fermentate of a bacterial strain does not comprise bacterial cells of the bacterial strain. For example, a fermentate may be separated from the bacterial cells that produced the fermentate (e.g., a culture supernatant). Bacterial cells may be separated from fermentate by any suitable method, such as centrifugation or filtration.

In some embodiments, a composition comprising a fermentate of a bacterial strain also comprises bacterial cells of the bacterial strain. In some embodiments, a composition does not comprise live bacterial cells. In some embodiments, the bacterial cells are heat-killed or pasteurized.

In some embodiments, one or more fermentates are in powder form. In some embodiments, each fermentate of a composition is in powder form. Powder is a dry, bulk solid comprised of many discrete, solid, macroscopic particles, which may flow freely when the powder (or a container in which the powder is present) is shaken or tilted. The skilled artisan will appreciate that powder form fermentates may be incorporated into a composition (e.g., with a carrier and in a defined form), where the composition as a whole has a solid form that is not a powder.

Any suitable method may be used to convert a bacterial fermentate to powder form. Non-limiting examples of powder production processes include grinding and agglomeration (e.g., of solid fermentates), crystallization and spray drying (e.g., of liquid fermentates). Advantages of converting a fermentate to powder form include, without limitation, ease of incorporation into a product intended for consumption (e.g., nutritional supplement or food product intended for ingestion or dental supplement intended for chewing), distribution throughout a product to limit the fermentate mixture's effect on taste and texture, and reduction of water activity to improve stability of desired components of a fermentate mixture.

In some embodiments, a bacterial fermentate mixture in powder form has a water content of 10% or less, 9% or less, 8% or less, 7% or less, 6% or less, 5% or less, 4% or less, 3% or less, 2% or less, or 1% or less.

In some embodiments, one or more fermentates are spray-dried or lyophilized. In some embodiments, one or more fermentates are spray-dried. In some embodiments, each fermentate of a composition is spray-dried. In some embodiments, one or more fermentates are lyophilized. In some embodiments, each fermentate of a composition is lyophilized. Where a fermentate is lyophilized and in powder form, powder may be lyophilized, or a lyophilized non-powder solid may be made into a powder form (e.g., by grinding and/or agglomeration).

Methods of lyophilizing compositions, specifically compositions comprising bacteria, are known in the art. See, e.g., U.S. Pat. Nos. 3,261,761; 4,205,132; and PCT Publication Nos. WO 2014/029578 and WO 2012/098358, herein incorporated by reference in their entirety. Fermentates may be lyophilized as a combination and/or the fermentates may be lyophilized separately and combined prior to incorporation into a composition, nutritional supplement, dental supplement, food product, and/or oral care product. A fermentate may be combined with a nutrient or carrier prior to combining it with another fermentate or multiple lyophilized fermentates may be combined while in lyophilized form and the mixture of fermentates, once combined may subsequently be combined with a nutrient and/or excipient. In some embodiments, the fermentate or combination thereof is a lyophilized cake. In such embodiments, the fermentate(s) may be rehydrated or suspended and/or cultured prior to use. In some embodiments, the fermentate(s) in lyophilized form are used directly, for example, without rehydrating or suspension (e.g., directly added to a medium).

The process of spray-drying refers to production of a dry powder from a liquid comprising a component, such as a fermentate. See, e.g., Ledet et al., Spray-Drying of Pharmaceuticals in “Lyophilized Biologics and Vaccines” pages 273-194, Springer. In general, the process involves rapidly drying the fermentates with a hot gas. A fermentate may be combined with an excipient or nutrient prior to combining it with other fermentates, or multiple spray-dried fermentates may be combined while in spray-dried form and the mixture of fermentates, once combined, may be subsequently combined with an excipient, carrier, or nutrient.

The amount of fermentate in a composition is quantified according to dry weight of the fermentate. In some embodiments, a fermentate is present in a composition at an amount of 10 to 500 mg. In some embodiments, a fermentate is present in a composition at an amount of 10 to 450, 10 to 400, 10 to 350, 10 to 300, 10 to 250, 10 to 200, 10 to 150, 10 to 100, or 10 to 50 mg. In some embodiments, a fermentate is present in a composition at an amount of 50 to 500, 100 to 500, 150 to 500, 200 to 500, 250 to 500, 300 to 500, 350 to 500, 400 to 500, or 450 to 500 mg. In some embodiments, a fermentate is present in a composition at an amount of 10 to 50, 50 to 100, 100 to 150, 150 to 200, 200 to 250, 250 to 300, 300 to 350, 350 to 400, 400 to 450, or 450 to 500 mg.

In some embodiments, the total mass of fermentates in a composition is 10 to 500 mg. In some embodiments, the total mass of fermentates in a composition is 10 to 450, 10 to 400, 10 to 350, 10 to 300, 10 to 250, 10 to 200, 10 to 150, 10 to 100, or 10 to 50 mg. In some embodiments, the total mass of fermentates in a composition is 50 to 500, 100 to 500, 150 to 500, 200 to 500, 250 to 500, 300 to 500, 350 to 500, 400 to 500, or 450 to 500 mg. In some embodiments, the total mass of fermentates in a composition is 10 to 50, 50 to 100, 100 to 150, 150 to 200, 200 to 250, 250 to 300, 300 to 350, 350 to 400, 400 to 450, or 450 to 500 mg.

### Nutritional Supplements, Dental Supplements, Food Products, and Oral Care Products

Some aspects relate to a nutritional supplement comprising any of the compositions or fermentates described herein. Some aspects relate to a dental supplement comprising any of the compositions or fermentates described herein. Some aspects relate to a food product comprising any of the compositions or fermentates described herein. Some aspects relate to an oral care product comprising any of the compositions or fermentates described herein. Food products and nutritional supplements are, in general, intended for the consumption of a human or other animal, such as a companion animal (e.g., a dog), or a bird. As will be appreciated by one of ordinary skill in the art, a food product provides a primary caloric and nutritive source intended for consumption by a subject (e.g., a feed or treat). In contrast, a nutritional supplement is not intended to provide a primary caloric or nutritive source for a subject but rather provides a targeted effect. Nutritional supplements may provide additional nutrients or functional ingredients that complement a standard diet, e.g., a standard pet diet. Dental supplements may be consumed, or be chewed without consuming (e.g., chewed and then spit out), by a human or other animal. Dental supplements are useful, for example, to exercise an animal's teeth and jaws, and to remove bacterial biofilm, plaque, tartar, and/or dental calculus. Any of the bacterial strains, fermentates thereof, or combinations thereof described herein may be present in a nutritional supplement, dental supplement, or food product. The compositions disclosed herein can be used in a food or beverage, such as a health food, a pet food, a functional food, a dietary supplement, a food or beverage for patients, or an animal feed.

Non-limiting examples of the forms of nutritional and/or dental supplements include carbohydrate-containing foods such as rice food products; paste products such as fish hams, sausages, paste products of seafood; pouch products. Further, the examples also include food products, nutritional supplements, and dental supplements prepared in the forms of powders, granules, kibbles, tablets, capsules, liquids, pastes, chews, and jellies.

Any suitable form may be used for a food product, nutritional supplement, or dental supplement. Non-limiting examples of form factors include dental sticks, dental chews, chews with softer interiors than exteriors, kibbles, and spreads. Additional examples of form factors include a gelatinized starch matrix, extruded or injection-molded dental stick, extruded or injection-molded dental chew, chew having a soft interior, powder meal topper, water additive, dry kibble or food, wet food, frozen food, liquid spray, peanut butter spread, and soft jerky chew.

In some embodiments, the nutritional or dental supplement is an extruded dental stick. In some embodiments, the nutritional or dental supplement is an injection-molded dental stick. In some embodiments, the nutritional or dental supplement is a chew having a soft interior. In some embodiments, the food product, nutritional supplement or dental supplement is a kibble. In some embodiments, the kibble is an extruded kibble. Extruded and/or injection-molded chews, sticks, and kibbles are known in the art, and any suitable method may be used to manufacture such chews, sticks, and kibbles.

In some embodiments, the food product, nutritional supplement or dental supplement is in the form of a wet food. In some embodiments, the food product, nutritional supplement or dental supplement is in the form of a frozen food. Wet foods and frozen foods may be in any suitable form, such as those described in Niamnuy, C., & Devahastin, S. (2010). 11 Pet Foods and Their Physicochemical Properties as Affected by Processing. Contemporary Food Engineering, 327.

In some embodiments, the food product, nutritional supplement, or dental supplement is a soft jerky chew. Jerky chews may be made from any suitable starting material, such as dried meat. In some embodiments, the food product, nutritional supplement, or dental supplement is a spread. In some embodiments, the spread comprises one or more nuts. In some embodiments, the spread is a peanut butter spread. The skilled artisan will appreciate that a food product or supplement intended for consumption by a particular animal will not contain ingredients that are toxic to that animal (e.g., macadamia nuts for dogs).

In some embodiments, the nutritional supplement is an additive. Additives may be added to other foods or food products before consumption by an animal. In some embodiments, the nutritional supplement is a water additive. Water additives are added to drinking water. In some embodiments, the nutritional supplement is a meal topper. Meal toppers may be added directly to food before consumption, with or without incorporation before consumption. In some embodiments, the meal topper is in powder form (a powder meal topper). In some embodiments, the nutritional supplement is a liquid spray. Liquid sprays may be administered directly to an animal, or sprayed on food or in water, with or without incorporation before consumption.

In some embodiments, the nutritional supplement, dental supplement, or food product comprises a fermentate or combination thereof, and a carrier. The carrier may contain any combination of nutrients, excipients, diluents, and/or binders. In some embodiments, the carrier comprises one or more of the following compounds: water, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, glucose, sucrose, sorbitol, lactose, whey, whey permeate, wheat flour, wheat bran, corn gluten meal, starch and cellulose. In some embodiments, the carrier is a gelatinized starch matrix. In some embodiments, the carrier is maltodextrin. In some embodiments, the carrier is a tapioca-based maltodextrin. Any suitable method may be used to obtain maltodextrin from tapioca, such as isolation from a tapioca plant.

In some embodiments, the carrier is present in a 2:1, 3:1, 4:1, 5:1, 6:1, 7:1, 8:1, 9:1, or 10:1 ratio by weight relative to the total amount of fermentates. In some embodiments, the carrier is present in a 1:2, 1:3, 1:4, 1:5, 1:6, 1:7, 1:8, 1:9, or 1:10 ratio by weight relative to the total amount of fermentates. In some embodiments, the carrier is present in a 2:1 ratio by weight relative to the total amount of fermentates. In some embodiments, the carrier is present in a 1:1 ratio by weight relative to the total amount of fermentates. In some embodiments, the carrier is present in a 2:1 ratio by weight relative to each fermentate. In some embodiments, the carrier is present in a 1:1 ratio by weight relative to each fermentate. In some embodiments, the carrier is present in a 1:1 ratio by weight relative to each of three fermentates. In some embodiments, the carrier is present in a 2:1 ratio by weight relative to each of two fermentates.

In some embodiments, a nutritional supplement, dental supplement, food product, and/or oral care product comprises a composition comprising fermentate(s) at an incorporation rate of 0.01% to 20% w/w. As used herein, an “incorporation rate” refers to the weight of the fermentate-containing composition as a percentage of the total weight of the nutritional supplement, or food product. For example, 10 mg of a fermentate-containing composition in a 50 g supplement is present at an incorporation rate of 0.2% w/w. In some embodiments, the incorporation rate is 0.01% to 20% w/w. In some embodiments, the incorporation rate is 0.01% to 15% w/w. In some embodiments, the incorporation rate is 0.01% to 10% w/w. In some embodiments, the incorporation rate is 0.01% to 5% w/w. In some embodiments, the incorporation rate is 0.1% to 10% w/w. In some embodiments, the incorporation rate is 0.1% to 2% w/w.

In some embodiments, the incorporation rate is 0.02% to 10%, 0.03% to 10%, 0.04% to 10%, 0.05% to 10%, 0.06% to 10%, 0.07% to 10%, 0.08% to 10%, 0.09% to 10%, 0.1% to 10%, 0.2% to 10%, 0.3% to 10%, 0.4% to 10%, 0.5% to 10%, 0.6% to 10%, 0.7% to 10%, 0.8% to 10%, 0.9% to 10%, 1% to 10%, 2% to 10%, 3% to 10%, 4% to 10%, 5% to 10%, 6% to 10%, 7% to 10%, 8% to 10%, or 9% to 10% w/w. In some embodiments, the incorporation rate is 0.1% to 10%, 0.1% to 9%, 0.1% to 8%, 0.1% to 7%, 0.1% to 6%, 0.1% to 5%, 0.1% to 4%, 0.1% to 3%, 0.1% to 2%, or 0.1% to 1% w/w. In some embodiments, the incorporation rate is 0.1% to 2%, 2% to 4%, 4% to 6%, 6% to 8%, 8% to 10%, 10% to 15%, or 15% to 20% w/w. In some embodiments, a nutritional supplement, dental supplement, food product, and/or oral care product comprises a certain mass of a composition comprising fermentate(s). Defined masses of compositions are useful, for example, for supplements, food products, and/or oral care products that may be diluted in food or water before consumption. In some embodiments, 2 to 2,000 mg of a fermentate-containing composition is present in a nutritional supplement, dental supplement, food product, and/or oral care product. In some embodiments, 20 to 2,000, 20 to 1,800, 20 to 1,600, 20 to 1,400, 20 to 1,200, 20 to 1,000, 20 to 900, 20 to 800, 20 to 700, 20 to 600, 20 to 500, 20 to 400, 20 to 300, 20 to 200, 20 to 100, or 20 to 50 mg of the composition is present. In some embodiments, 50 to 2,000, 100 to 2,000, 200 to 2,000, 300 to 2,000, 400 to 2,000, 500 to 2,000, 600 to 2,000, 700 to 2,000, 800 to 2,000, 900 to 2,000, 1,000 to 2,000, 1,200 to 2,000, 1,400 to 2,000, 1,600 to 2,000, or 1,800 to 2,000 mg of the composition is present. In some embodiments, 100 to 200, 200 to 300, 300 to 400, 400 to 500, 500 to 600, 600 to 700, 700 to 800, 800 to 900, 1,000 to 1,200, 1,200 to 1,400, 1,400 to 1,600, 1,600 to 1,800, or 1,800 to 2,000 mg of the composition is present. In some embodiments, 0.01 to 0.1, 0.1 to 1, 1 to 10, 10 to 100, 100 to 1,000, or 1,000 to 10,000 mg of the composition is present.

A nutritional supplement, dental supplement, food product, and/or oral care product may comprise one or more nutrients. The nutrient may be a carbohydrate or carbohydrate source. Non-limiting examples of carbohydrate sources include grains, such as corn, rice, milo, sorghum, barley, alfalfa, oats, and wheat. In some embodiments, the nutrient is a grain. In some embodiments, the nutrient is wheat, corn, rice, oats, and/or barley. A nutritional supplement, dental supplement, food product, and/or oral care product may comprise a gelatinized starch matrix. Matrices of gelatinized starch may comprise any suitable starch, such as a starch listed herein or another starch known in the art.

A nutritional supplement, dental supplement, food product, and/or oral care product may comprise a fiber. Fibers may add bulk to a composition and may be fermented by intestinal bacteria to produce short-chain fatty acids and other metabolites. Non-limiting examples of fibers include tapioca fiber, beet pulp (e.g., sugar beet pulp), gum arabic, gum talha, psyllium, rice bran, carob bean gum, citrus pulp, pectin, fructooligosaccharide, mannaoligofructose, soy fiber, arabinogalactan, galactooligosaccharide, arabinoxylan, chicory pulp, chicory, coconut endosperm fiber, wheat fiber, and inulin.

A nutritional supplement, dental supplement, food product, and/or oral care product may comprise an algal strain. Algae may add one or more nutrients and/or bulk to a composition. Non-limiting examples of algae include Ascophyllum nodosum, Spirulina (e.g., Arthrospira platensis, Arthrospira fusiformis, and Arthrospira maxima), Chlorella, Ulva lactuca, Laminaria digitata and Fucus vesiculosus.

A nutritional supplement, dental supplement, food product, and/or oral care product may comprise one or more inorganic minerals. Non-limiting examples of minerals include calcium carbonate, calcium, boron, selenium, calcium chloride, chloride, ferrous fumarate, zinc acetate, choline chloride, chromium, ferrous gluconate, zinc sulfate, chromium, tripicolinate, cobalt, magnesium oxide, zinc gluconate, dicalcium phosphate, copper, magnesium sulfate, ferrous sulfate, iodine, magnesium carbonate, monosodium phosphate, iron, chromium picolinate, potassium chloride, magnesium, calcium citrate, potassium citrate, manganese, calcium lactate, potassium sorbate, phosphorus, calcium gluconate, sodium bisulfate, potassium, chromium chloride, sodium hexametaphosphate, sodium, chromium nicotinate, tricalcium phosphate, zinc, and chromium citrate.

A nutritional supplement, dental supplement, food product, and/or oral care product may comprise one or more protein sources. Non-limiting examples of such protein sources include chicken meals, chicken, chicken byproduct meals, eggs, lamb, lamb meals, turkey, turkey meals, beef, beef by-products, viscera, fish, fish meal, enterals, kangaroo, white fish, venison, blood marrow, bone marrow, soybean meal, soy protein isolate, and soy protein concentrate.

A nutritional supplement, dental supplement, food product, and/or oral care product may comprise one or more starches. Non-limiting examples of such starches include cereals, grains, corn, wheat, rice, oats, corn grits, sorghum, grain sorghum, milo, wheat bran, oat bran, amaranth, durum, and semolina.

A nutritional supplement, dental supplement, food product, and/or oral care product may comprise one or more dairy products. Dairy products include, without limitation, cream, milk, butter, and cheese.

A nutritional supplement, dental supplement, food product, and/or oral care product may comprise a meat or animal-derived material (e.g., animal-derived protein source). In some embodiments, the meat or animal-derived material is beef, chicken, eggs, turkey, lamb, fish, blood marrow, bone marrow, or a combination of any thereof.

An oral care product may be in any form suitable for delivery of a composition (e.g., one or more fermentates) to the oral cavity. In some embodiments, an oral care product is a toothpaste. In some embodiments, an oral care product is a mouthwash. In some embodiments, an oral care product is a breath mint. In some embodiments, an oral care product is a chewing gum.

### Methods of Use

Some aspects of the present disclosure relate to administration of a composition (and/or nutritional supplement, dental supplement, food product, and/or oral care product comprising the same) to a subject in need thereof. Administration may be accomplished through any suitable method, such as one described herein or known in the art, and may include, for example, consumption of the composition (and/or nutritional supplement, dental supplement, food product, and/or oral care product) by the subject.

In some embodiments, administration is oral. Oral administration may refer to manual administration, such as oral gavage or spraying (e.g., via aerosol). Oral administration may also refer to feeding, such as providing the composition or nutritional supplement to a subject (e.g., a companion animal), which ingests it by eating or drinking.

In some embodiments, administration is by application to the oral cavity. In some embodiments, oral administration is performed without ingesting the composition. In some embodiments, oral administration comprises maintaining the presence of the composition in the oral cavity and releasing the composition from the oral cavity without swallowing the composition. In some embodiments, oral administration comprises maintaining the presence of the composition in the oral cavity until it is dissolved. In some embodiments, oral administration comprises maintaining the presence of composition in the oral cavity for at least 30 seconds, at least 1 minute, at least 2 minutes, at least 3 minutes, at least 4 minutes, at least 5 minutes, at least 10 minutes, at least 15 minutes, at least 20 minutes, or at least 30 minutes. In some embodiments, the composition is applied using a toothbrush. In some embodiments, oral administration comprises chewing the composition and not swallowing.

Methods may include a single administration or administration may be repeated. Repeat administrations may be conducted at regular intervals (e.g., daily or weekly). For example, a subject may be administered a composition or supplement one or more times daily, over a specified time period, such as a week, several weeks, a month, or several months. Alternatively, multiple instances of administration may be conducted on an as-needed basis.

In some embodiments, administration is rectal. In some embodiments, a composition, supplement, or food product is in the form of a suppository when administered rectally. In some embodiments, a composition, supplement, or food product is delivered to the intestine. In some embodiments, a composition, supplement, or food product is delivered to the colon. Such delivery to the intestine or colon may be accomplished by oral or rectal administration.

In some embodiments, a composition, nutritional supplement, dental supplement, or food product is administered in an effective amount for treating, preventing, or alleviating one or more symptoms in a subject. An “effective amount” or “effective dosage” refers to an amount or dosage, respectively, sufficient to treat, alleviate, or prevent a sign or symptom in a subject. The actual effective amount or dosage will depend on the individual subject and their health. Such effective amounts or dosages may be determined by routine assays known to those of skill in the art.

Subjects to which a composition, supplement, or food product may be administered include any animal known in the art. In some embodiments, the subject is a human. In some embodiments, the animal is a domesticated animal. In some embodiments, the animal is a carnivore. In some embodiments, the animal is a rodent. In some embodiments, the rodent is a mouse, rat, guinea pig, chinchilla, or hamster. In some embodiments, the animal is a dog, cat, rabbit, guinea pig, hamster, or ferret. In some embodiments, the animal is a dog. In some embodiments, the animal is a bovine, swine, llama, alpaca, sheep, or goat. In some embodiments, the animal is a bird.

In some embodiments, the composition, nutritional supplement, dental supplement, food product, and/or oral care product is effective in reducing one or more symptoms, such as bad breath, gingivitis, dental calculus buildup, inflammation, diarrhea, vomiting, itching, constipation, lack of appetite, lethargy, arthritis, excessive grooming, abdominal pain, and fever. In some embodiments, the composition, nutritional supplement, dental supplement, food product, and/or oral care product treats or prevents bad breath in the subject. In some embodiments, the composition, nutritional supplement, dental supplement, food product, and/or oral care product treats or prevents gingivitis in the subject. In some embodiments, the composition, nutritional supplement, dental supplement, food product, and/or oral care product treats or prevents dental calculus buildup in the subject. In some embodiments, the composition, nutritional supplement, dental supplement, food product, and/or oral care product treats or prevents diarrhea in the subject. In some embodiments, the composition, nutritional supplement, dental supplement, food product, and/or oral care product treats or prevents vomiting or nausea in the subject.

In some embodiments, a composition, nutritional supplement, dental supplement, food product, and/or oral care product is administered to a subject having halitosis. Signs and symptoms of halitosis include unpleasant breath containing one or more volatile sulfur compounds, gingivitis, and accumulation of plaque, tartar, and/or dental calculus on teeth of the subject. In some embodiments, the oral microenvironment of the subject comprises one or more microorganisms associated with halitosis. Exemplary microorganisms associated with halitosis include, without limitation, Porphyromonas gingivalis, Prevotella intermedia, Porphyromonas canigingivalis, Tannerella forsythia, Treponema denticola, Fusobacterium nucleatum, Neisseria canis, and Bergeyella zoohelcum. In some embodiments, the subject's oral microenvironment comprises Porphyromonas gingivalis. In some embodiments, the subject's oral microenvironment comprises Prevotella intermedia. In some embodiments, the subject's oral microenvironment comprises Porphyromonas canigingivalis. In some embodiments, the subject's oral microenvironment comprises Tannerella forsythia. In some embodiments, the subject's oral microenvironment comprises Treponema denticola. In some embodiments, the subject's oral microenvironment comprises Fusobacterium nucleatum. In some embodiments, the subject's oral microenvironment comprises Neisseria canis. In some embodiments, the subject's oral microenvironment comprises Bergeyella zoohelcum.

Any suitable method may be used to determine whether a subject's oral microenvironment comprises a given microorganism. For example, culture-based methods, microscopy-based methods, 16S rDNA sequencing, and/or whole-genome sequencing-based methods may be performed on a sample obtained from a subject's oral microenvironment. In some embodiments, one or more microorganisms is present in a saliva sample. In some embodiments, one or more microorganisms is present on oral mucosa of a subject. In some embodiments, one or more microorganisms is present on the tongue of a subject. In some embodiments, one or more microorganisms is present on a tooth of a subject.

In some embodiments, the composition, nutritional supplement, dental supplement, food product, and/or oral care product is effective in treating or preventing one or more symptoms or adverse events in a subject. As discussed above, the compositions reduce VSC abundance, inhibit VSC production, and/or reduce an alkaline oral environment, thereby improving halitosis. In some embodiments, the composition, nutritional supplement, dental supplement, food product, and/or oral care product reduces VSC abundance in the subject. Methods of measuring VSC abundance are known in the art, and include, without limitation, measuring the concentration of one or more VSCs (e.g., hydrogen sulfide, methyl mercaptan, and dimethyl sulfide) in the subject, such as in a sample obtained from the oral microenvironment (e.g., a breath or saliva sample).

In some embodiments, the composition, nutritional supplement, dental supplement, food product, and/or oral care product reduces inflammation in the subject. In some embodiments, the composition, nutritional supplement, dental supplement, food product, and/or oral care product treats or prevents one or more symptoms associated with inflammation. Methods of measuring inflammation are known in the art, and include, without limitation, measuring the concentration of one or more cytokines (e.g., TNF-α, IL-1β, IL-6, IL-10, and IL-12) in the subject, such as in a sample obtained from the subject (e.g., a blood sample, serum sample, plasma sample). In some embodiments, the composition, nutritional supplement, dental supplement, food product, and/or oral care product reduces infiltration of leukocytes (e.g., neutrophils and/or monocytes) into oral mucosa. In some embodiments, the composition, nutritional supplement, dental supplement, food product, and/or oral care product reduces the rate of plaque accumulation, compared to a reference subject (e.g., a subject that has not been administered the composition or nutritional supplement). In some embodiments, the composition, nutritional supplement, dental supplement, food product, and/or oral care product reduces the rate of tartar accumulation, compared to a reference subject (e.g., a subject that has not been administered the composition or nutritional supplement). In some embodiments, the composition, nutritional supplement, dental supplement, food product, and/or oral care product reduces the rate of dental calculus accumulation, compared to a reference subject (e.g., a subject that has not been administered the composition or nutritional supplement).

In some embodiments, administration results in a decrease in abundance of dental plaque by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of dental plaque prior to administering the composition. In some embodiments, administration of a composition results in decrease in the abundance of dental plaque by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of dental plaque in a subject (e.g., a reference subject) who did not receive the composition.

In some embodiments, administration results in a decrease in abundance of dental tartar by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of dental tartar prior to administering the composition. In some embodiments, administration of a composition results in decrease in the abundance of dental tartar by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of dental tartar in a subject (e.g., a reference subject) who did not receive the composition.

In some embodiments, administration results in a decrease in abundance of dental calculus by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of dental calculus prior to administering the composition. In some embodiments, administration of a composition results in decrease in the abundance of dental calculus by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of dental calculus in a subject (e.g., a reference subject) who did not receive the composition.

In some embodiments, administration of a composition results in a decrease in abundance of one or more microorganisms in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of one or more microorganisms in the subject (or microbiome thereof) prior to administering the composition. In some embodiments, administration of the composition results in a decrease in abundance of one or more microorganisms in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of one or more microorganisms in another subject (e.g., a reference subject) (or microbiome thereof) who did not receive the composition.

In some embodiments, administration results in a decrease in abundance of one or more microorganisms in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of the microorganism in the subject (or microbiome thereof) prior to administering the composition. In some embodiments, administration of a composition results in decrease in the abundance of one or more microorganisms in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of the microorganisms in a subject (e.g., a reference subject) (or microbiome thereof) who did not receive the composition.

In some embodiments, administration of a composition results in a decrease in abundance of Porphyromonas gingivalis in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Porphyromonas gingivalis in the subject (or microbiome thereof) prior to administering the composition. In some embodiments, administration of the composition results in a decrease in abundance of Porphyromonas gingivalis in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Porphyromonas gingivalis in another subject (e.g., a reference subject) (or microbiome thereof) who did not receive the composition.

In some embodiments, administration results in a decrease in abundance of Porphyromonas gingivalis in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Porphyromonas gingivalis in the subject (or microbiome thereof) prior to administering the composition. In some embodiments, administration of a composition results in decrease in the abundance of Porphyromonas gingivalis in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Porphyromonas gingivalis in a subject (e.g., a reference subject) (or microbiome thereof) who did not receive the composition.

In some embodiments, administration of a composition results in a decrease in abundance of Prevotella intermedia in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Prevotella intermedia in the subject (or microbiome thereof) prior to administering the composition. In some embodiments, administration of the composition results in a decrease in abundance of Prevotella intermedia in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Prevotella intermedia in another subject (e.g., a reference subject) (or microbiome thereof) who did not receive the composition.

In some embodiments, administration results in a decrease in abundance of Prevotella intermedia in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Prevotella intermedia in the subject (or microbiome thereof) prior to administering the composition. In some embodiments, administration of a composition results in decrease in the abundance of Prevotella intermedia in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Prevotella intermedia in a subject (e.g., a reference subject) (or microbiome thereof) who did not receive the composition.

In some embodiments, administration of a composition results in a decrease in abundance of Porphyromonas canigingivalis in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Porphyromonas canigingivalis in the subject (or microbiome thereof) prior to administering the composition. In some embodiments, administration of the composition results in a decrease in abundance of Porphyromonas canigingivalis in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Porphyromonas canigingivalis in another subject (e.g., a reference subject) (or microbiome thereof) who did not receive the composition.

In some embodiments, administration results in a decrease in abundance of Porphyromonas canigingivalis in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Porphyromonas canigingivalis in the subject (or microbiome thereof) prior to administering the composition. In some embodiments, administration of a composition results in decrease in the abundance of Porphyromonas canigingivalis in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Porphyromonas canigingivalis in a subject (e.g., a reference subject) (or microbiome thereof) who did not receive the composition.

In some embodiments, administration of a composition results in a decrease in abundance of Tannerella forsythia in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Tannerella forsythia in the subject (or microbiome thereof) prior to administering the composition. In some embodiments, administration of the composition results in a decrease in abundance of Tannerella forsythia in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Tannerella forsythia in another subject (e.g., a reference subject) (or microbiome thereof) who did not receive the composition.

In some embodiments, administration results in a decrease in abundance of Tannerella forsythia in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Tannerella forsythia in the subject (or microbiome thereof) prior to administering the composition. In some embodiments, administration of a composition results in decrease in the abundance of Tannerella forsythia in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Tannerella forsythia in a subject (e.g., a reference subject) (or microbiome thereof) who did not receive the composition.

In some embodiments, administration of a composition results in a decrease in abundance of Treponema denticola in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Treponema denticola in the subject (or microbiome thereof) prior to administering the composition. In some embodiments, administration of the composition results in a decrease in abundance of Treponema denticola in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Treponema denticola in another subject (e.g., a reference subject) (or microbiome thereof) who did not receive the composition.

In some embodiments, administration results in a decrease in abundance of Treponema denticola in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Treponema denticola in the subject (or microbiome thereof) prior to administering the composition. In some embodiments, administration of a composition results in decrease in the abundance of Treponema denticola in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Treponema denticola in a subject (e.g., a reference subject) (or microbiome thereof) who did not receive the composition.

In some embodiments, administration of a composition results in a decrease in abundance of Fusobacterium nucleatum in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Fusobacterium nucleatum in the subject (or microbiome thereof) prior to administering the composition. In some embodiments, administration of the composition results in a decrease in abundance of Fusobacterium nucleatum in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Fusobacterium nucleatum in another subject (e.g., a reference subject) (or microbiome thereof) who did not receive the composition.

In some embodiments, administration results in a decrease in abundance of Fusobacterium nucleatum in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Fusobacterium nucleatum in the subject (or microbiome thereof) prior to administering the composition. In some embodiments, administration of a composition results in decrease in the abundance of Fusobacterium nucleatum in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Fusobacterium nucleatum in a subject (e.g., a reference subject) (or microbiome thereof) who did not receive the composition.

In some embodiments, administration of a composition results in a decrease in abundance of Neisseria canis in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Neisseria canis in the subject (or microbiome thereof) prior to administering the composition. In some embodiments, administration of the composition results in a decrease in abundance of Neisseria canis in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Neisseria canis in another subject (e.g., a reference subject) (or microbiome thereof) who did not receive the composition.

In some embodiments, administration results in a decrease in abundance of Neisseria canis in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Neisseria canis in the subject (or microbiome thereof) prior to administering the composition. In some embodiments, administration of a composition results in decrease in the abundance of Neisseria canis in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Neisseria canis in a subject (e.g., a reference subject) (or microbiome thereof) who did not receive the composition.

In some embodiments, administration of a composition results in a decrease in abundance of Bergeyella zoohelcum in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Bergeyella zoohelcum in the subject (or microbiome thereof) prior to administering the composition. In some embodiments, administration of the composition results in a decrease in abundance of Bergeyella zoohelcum in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Bergeyella zoohelcum in another subject (e.g., a reference subject) (or microbiome thereof) who did not receive the composition.

In some embodiments, administration results in a decrease in abundance of Bergeyella zoohelcum in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Bergeyella zoohelcum in the subject (or microbiome thereof) prior to administering the composition. In some embodiments, administration of a composition results in decrease in the abundance of Bergeyella zoohelcum in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Bergeyella zoohelcum in a subject (e.g., a reference subject) (or microbiome thereof) who did not receive the composition.

In some embodiments, administration reduces the concentration of 1, 2, 3, 4, 5, 6, 7, 8, 9, 10, 11, 12, 13, 14, 15, 16, 17, 18, 19, or 20 VSCs with distinct structures in the oral microenvironment of the subject. In some embodiments, administration reduces the concentration of 1, 2, or 3 VSCs with distinct structures. In some embodiments, administration reduces the concentration of hydrogen sulfide. In some embodiments, administration reduces the concentration of methyl mercaptan. In some embodiments, administration reduces the concentration of dimethyl sulfide.

In some embodiments, administration reduces the concentration of VSCs in an oral microenvironment by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, administration reduces the concentration of VSCs by 5-10%, 10-20%, 20-30%, 30-40%, 40-50%, 50-60%, 60-70%, 70-80%, 80-90%, or 90-100%. In some embodiments, administration reduces the concentration of VSCs by 5-95%, 5-90%, 5-80%, 5-70%, 5-60%, 5-50%, 5-40%, 5-30%, 5-25%, 5-20%, 5-15%, or 5-10%. In some embodiments, administration reduces the concentration of VSCs by 5-99%, 10-99%, 15-99%, 20-99%, 25-99%, 30-99%, 40-99%, 50-99%, 60-99%, 70-99%, 80-99%, or 90-99%.

In some embodiments, administration reduces the concentration of hydrogen sulfide in an oral microenvironment by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, administration reduces the concentration of hydrogen sulfide by 5-10%, 10-20%, 20-30%, 30-40%, 40-50%, 50-60%, 60-70%, 70-80%, 80-90%, or 90-100%. In some embodiments, administration reduces the concentration of hydrogen sulfide by 5-95%, 5-90%, 5-80%, 5-70%, 5-60%, 5-50%, 5-40%, 5-30%, 5-25%, 5-20%, 5-15%, or 5-10%. In some embodiments, administration reduces the concentration of hydrogen sulfide by 5-99%, 10-99%, 15-99%, 20-99%, 25-99%, 30-99%, 40-99%, 50-99%, 60-99%, 70-99%, 80-99%, or 90-99%.

In some embodiments, administration reduces the concentration of methyl mercaptan in an oral microenvironment by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, administration reduces the concentration of methyl mercaptan by 5-10%, 10-20%, 20-30%, 30-40%, 40-50%, 50-60%, 60-70%, 70-80%, 80-90%, or 90-100%. In some embodiments, administration reduces the concentration of methyl mercaptan by 5-95%, 5-90%, 5-80%, 5-70%, 5-60%, 5-50%, 5-40%, 5-30%, 5-25%, 5-20%, 5-15%, or 5-10%. In some embodiments, administration reduces the concentration of methyl mercaptan by 5-99%, 10-99%, 15-99%, 20-99%, 25-99%, 30-99%, 40-99%, 50-99%, 60-99%, 70-99%, 80-99%, or 90-99%.

In some embodiments, administration reduces the concentration of dimethyl sulfide in an oral microenvironment by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, administration reduces the concentration of dimethyl sulfide by 5-10%, 10-20%, 20-30%, 30-40%, 40-50%, 50-60%, 60-70%, 70-80%, 80-90%, or 90-100%. In some embodiments, administration reduces the concentration of dimethyl sulfide by 5-95%, 5-90%, 5-80%, 5-70%, 5-60%, 5-50%, 5-40%, 5-30%, 5-25%, 5-20%, 5-15%, or 5-10%. In some embodiments, administration reduces the concentration of dimethyl sulfide by 5-99%, 10-99%, 15-99%, 20-99%, 25-99%, 30-99%, 40-99%, 50-99%, 60-99%, 70-99%, 80-99%, or 90-99%.

The extent to which a composition or nutritional supplement treats or prevents a symptom may be evaluated using methods known in the art, such as monitoring the change in the frequency, occurrence, and/or severity of symptoms following administration of the composition or nutritional supplement. Additionally, groups of subjects administered the bacterial mixture, composition, or nutritional supplement may be monitored and compared to another group of subjects that was not administered the composition or nutritional supplement, a group that was administered a different amount of the composition or nutritional supplement, and/or a group that was administered a control composition (e.g., a carrier or excipient lacking the fermentate(s)).

Reduction of Volatile Sulfur Compounds (VSCs) Abundance and/or Inhibition of VSC Production

Fermentates of bacterial strains for use in the compositions described herein were identified based on the ability to reduce the abundance of volatile sulfur compounds (VSCs) in an environment and/or inhibit the production of VSCs by other microorganisms in the environment. VSCs are associated with the unpleasant odor of breath of subjects with halitosis, and reduction in VSC abundance (e.g., by VSC metabolism or inhibition of VSC production) is expected to alleviate this symptom of halitosis. Non-limiting examples of such VSCs include hydrogen sulfide, methyl mercaptan, and dimethyl sulfide, all of which give off strong and unpleasant odors.

In some embodiments, a composition, nutritional supplement, dental supplement, food product, and/or oral care product reduces the abundance of one or more microorganisms associated with halitosis in the subject or the subject's oral microenvironment. In some embodiments, a composition, nutritional supplement, dental supplement, food product, and/or oral care product alleviates one or more signs and symptoms of halitosis in a subject. Signs and symptoms of halitosis include unpleasant breath containing one or more volatile sulfur compounds, gingivitis, and accumulation of plaque, tartar, and/or dental calculus on teeth of the subject.

In some embodiments, a composition comprising a fermentate (and/or a nutritional supplement, dental supplement, food product, and/or oral care product comprising the same) reduces the concentration of 1, 2, 3, 4, 5, 6, 7, 8, 9, 10, 11, 12, 13, 14, 15, 16, 17, 18, 19, or 20 VSCs with distinct structures. In some embodiments, the composition reduces the concentration of 1, 2, or 3 VSCs with distinct structures. In some embodiments, the composition reduces the concentration of hydrogen sulfide. In some embodiments, the composition reduces the concentration of methyl mercaptan. In some embodiments, the composition reduces the concentration of dimethyl sulfide.

In some embodiments, the composition reduces the concentration of VSCs by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, the composition reduces the concentration of VSCs by 5-10%, 10-20%, 20-30%, 30-40%, 40-50%, 50-60%, 60-70%, 70-80%, 80-90%, or 90-100%. In some embodiments, the composition reduces the concentration of VSCs by 5-95%, 5-90%, 5-80%, 5-70%, 5-60%, 5-50%, 5-40%, 5-30%, 5-25%, 5-20%, 5-15%, or 5-10%. In some embodiments, the composition reduces the concentration of VSCs by 5-99%, 10-99%, 15-99%, 20-99%, 25-99%, 30-99%, 40-99%, 50-99%, 60-99%, 70-99%, 80-99%, or 90-99%.

In some embodiments, the composition reduces the concentration of hydrogen sulfide by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, the composition reduces the concentration of hydrogen sulfide by 5-10%, 10-20%, 20-30%, 30-40%, 40-50%, 50-60%, 60-70%, 70-80%, 80-90%, or 90-100%. In some embodiments, the composition reduces the concentration of hydrogen sulfide by 5-95%, 5-90%, 5-80%, 5-70%, 5-60%, 5-50%, 5-40%, 5-30%, 5-25%, 5-20%, 5-15%, or 5-10%. In some embodiments, the composition reduces the concentration of hydrogen sulfide by 5-99%, 10-99%, 15-99%, 20-99%, 25-99%, 30-99%, 40-99%, 50-99%, 60-99%, 70-99%, 80-99%, or 90-99%.

In some embodiments, the composition reduces the concentration of methyl mercaptan by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, the composition reduces the concentration of methyl mercaptan by 5-10%, 10-20%, 20-30%, 30-40%, 40-50%, 50-60%, 60-70%, 70-80%, 80-90%, or 90-100%. In some embodiments, the composition reduces the concentration of methyl mercaptan by 5-95%, 5-90%, 5-80%, 5-70%, 5-60%, 5-50%, 5-40%, 5-30%, 5-25%, 5-20%, 5-15%, or 5-10%. In some embodiments, the composition reduces the concentration of methyl mercaptan by 5-99%, 10-99%, 15-99%, 20-99%, 25-99%, 30-99%, 40-99%, 50-99%, 60-99%, 70-99%, 80-99%, or 90-99%.

In some embodiments, the composition reduces the concentration of dimethyl sulfide by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, the composition reduces the concentration of dimethyl sulfide by 5-10%, 10-20%, 20-30%, 30-40%, 40-50%, 50-60%, 60-70%, 70-80%, 80-90%, or 90-100%. In some embodiments, the composition reduces the concentration of dimethyl sulfide by 5-95%, 5-90%, 5-80%, 5-70%, 5-60%, 5-50%, 5-40%, 5-30%, 5-25%, 5-20%, 5-15%, or 5-10%. In some embodiments, the composition reduces the concentration of dimethyl sulfide by 5-99%, 10-99%, 15-99%, 20-99%, 25-99%, 30-99%, 40-99%, 50-99%, 60-99%, 70-99%, 80-99%, or 90-99%.

Not all fermentates must reduce, or be produced by bacterial strains that reduce, the concentration of VSCs. Some bacterial strains and fermentates thereof may (i) produce one or more antioxidants; (ii) enhance the ability of other bacterial strains or fermentates thereof to reduce VSC concentration; (iii) inhibit VSC production by other microorganisms; and/or (iv) disrupt a biofilm of one or more oral microbes.

In some embodiments, a composition comprises one or more antioxidants. In some embodiments a fermentate comprises one or more antioxidants. In some embodiments, a fermentate is produced by a bacterial strain that produces one or more antioxidants.

In some embodiments, a composition comprises one or more organic acids. In some embodiments a fermentate comprises one or more organic acids. In some embodiments, a fermentate is produced by a bacterial strain that produces one or more organic acids. Non-limiting examples of organic acids include lactic acid, ascorbic acid, acetic acid, acrylic acid, propionic acid, pyruvic acid, and citric acid.

In some embodiments, a composition enhances the ability of one or more other fermentates to reduce the concentration of VSCs. In some embodiments, a fermentate enhances the ability of one or more other fermentates to reduce the concentration of VSCs. In some embodiments, a fermentate is produced by a bacterial strain that enhances the ability of one or more other fermentates or bacterial strains to reduce the concentration of VSCs.

In some embodiments, a composition inhibits VSC production by another microorganism. In some embodiments, a fermentate inhibits VSC production by another microorganism. In some embodiments, a fermentate is produced by a bacterial strain that inhibits VSC production by another microorganism. In some embodiments, VSC production by Porphyromonas gingivalis is inhibited. In some embodiments, VSC production by Prevotella intermedia is inhibited. In some embodiments, VSC production by Porphyromonas canigingivalis is inhibited. In some embodiments, VSC production by Tannerella forsythia is inhibited. In some embodiments, VSC production by Treponema denticola is inhibited. In some embodiments, VSC production by Fusobacterium nucleatum is inhibited. In some embodiments, VSC production by Neisseria canis is inhibited. In some embodiments, VSC production by Bergeyella zoohelcum is inhibited.

In some embodiments, a composition inhibits hydrogen sulfide production by another microorganism. In some embodiments, a fermentate inhibits hydrogen sulfide production by another microorganism. In some embodiments, a fermentate is produced by a bacterial strain that inhibits hydrogen sulfide production by another microorganism. In some embodiments, hydrogen sulfide production by Porphyromonas gingivalis is inhibited. In some embodiments, hydrogen sulfide production by Prevotella intermedia is inhibited. In some embodiments, hydrogen sulfide production by Porphyromonas canigingivalis is inhibited. In some embodiments, hydrogen sulfide production by Tannerella forsythia is inhibited. In some embodiments, hydrogen sulfide production by Treponema denticola is inhibited. In some embodiments, hydrogen sulfide production by Fusobacterium nucleatum is inhibited. In some embodiments, hydrogen sulfide production by Neisseria canis is inhibited. In some embodiments, hydrogen sulfide production by Bergeyella zoohelcum is inhibited.

In some embodiments, a composition inhibits methyl mercaptan production by another microorganism. In some embodiments, a fermentate inhibits methyl mercaptan production by another microorganism. In some embodiments, a fermentate is produced by a bacterial strain that inhibits methyl mercaptan production by another microorganism. In some embodiments, methyl mercaptan production by Porphyromonas gingivalis is inhibited. In some embodiments, methyl mercaptan production by Prevotella intermedia is inhibited. In some embodiments, methyl mercaptan production by Porphyromonas canigingivalis is inhibited. In some embodiments, methyl mercaptan production by Tannerella forsythia is inhibited. In some embodiments, methyl mercaptan production by Treponema denticola is inhibited. In some embodiments, methyl mercaptan production by Fusobacterium nucleatum is inhibited. In some embodiments, methyl mercaptan production by Neisseria canis is inhibited. In some embodiments, methyl mercaptan production by Bergeyella zoohelcum is inhibited.

In some embodiments, a composition inhibits dimethyl sulfide production by another microorganism. In some embodiments, a fermentate inhibits dimethyl sulfide production by another microorganism. In some embodiments, a fermentate is produced by a bacterial strain that inhibits dimethyl sulfide production by another microorganism. In some embodiments, dimethyl sulfide production by Porphyromonas gingivalis is inhibited. In some embodiments, dimethyl sulfide production by Prevotella intermedia is inhibited. In some embodiments, dimethyl sulfide production by Porphyromonas canigingivalis is inhibited. In some embodiments, dimethyl sulfide production by Tannerella forsythia is inhibited. In some embodiments, dimethyl sulfide production by Treponema denticola is inhibited. In some embodiments, dimethyl sulfide production by Fusobacterium nucleatum is inhibited. In some embodiments, dimethyl sulfide production by Neisseria canis is inhibited. In some embodiments, dimethyl sulfide production by Bergeyella zoohelcum is inhibited.

In some embodiments, a composition reduces the abundance in an oral cavity of a subject of one or more microorganisms that produce VSCs. In some embodiments, a fermentate reduces the abundance in an oral cavity of a subject of one or more microorganisms that produce VSCs. In some embodiments, a fermentate is produced by a bacterial strain that reduces the abundance in an oral cavity of a subject of one or more microorganisms that produce VSCs. In some embodiments, the abundance of Porphyromonas gingivalis is reduced. In some embodiments, the abundance of Prevotella intermedia is reduced. In some embodiments, the abundance of Porphyromonas canigingivalis is reduced. In some embodiments, the abundance of Tannerella forsythia is reduced. In some embodiments, the abundance of Treponema denticola is reduced. In some embodiments, the abundance of Fusobacterium nucleatum is reduced. In some embodiments, the abundance of Neisseria canis is reduced. In some embodiments, the abundance of Bergeyella zoohelcum is reduced.

In some embodiments, the abundance of Porphyromonas gingivalis in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Porphyromonas gingivalis in the oral cavity (or microbiome thereof) prior to exposure to the composition or fermentate(s). In some embodiments, the abundance of Porphyromonas gingivalis in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Porphyromonas gingivalis in the oral cavity (or microbiome thereof) prior to exposure to the composition or fermentate(s).

In some embodiments, the abundance of Prevotella intermedia in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Prevotella intermedia in the oral cavity (or microbiome thereof) prior to exposure to the composition or fermentate(s). In some embodiments, the abundance of Prevotella intermedia in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Prevotella intermedia in the oral cavity (or microbiome thereof) prior to exposure to the composition or fermentate(s).

In some embodiments, the abundance of Porphyromonas canigingivalis in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Porphyromonas canigingivalis in the oral cavity (or microbiome thereof) prior to exposure to the composition or fermentate(s). In some embodiments, the abundance of Porphyromonas canigingivalis in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Porphyromonas canigingivalis in the oral cavity (or microbiome thereof) prior to exposure to the composition or fermentate(s).

In some embodiments, the abundance of Tannerella forsythia in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Tannerella forsythia in the oral cavity (or microbiome thereof) prior to exposure to the composition or fermentate(s). In some embodiments, the abundance of Tannerella forsythia in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Tannerella forsythia in the oral cavity (or microbiome thereof) prior to exposure to the composition or fermentate(s).

In some embodiments, the abundance of Treponema denticola in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Treponema denticola in the oral cavity (or microbiome thereof) prior to exposure to the composition or fermentate(s). In some embodiments, the abundance of Treponema denticola in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Treponema denticola in the oral cavity (or microbiome thereof) prior to exposure to the composition or fermentate(s).

In some embodiments, the abundance of Fusobacterium nucleatum in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Fusobacterium nucleatum in the oral cavity (or microbiome thereof) prior to exposure to the composition or fermentate(s). In some embodiments, the abundance of Fusobacterium nucleatum in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Fusobacterium nucleatum in the oral cavity (or microbiome thereof) prior to exposure to the composition or fermentate(s).

In some embodiments, the abundance of Neisseria canis in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Neisseria canis in the oral cavity (or microbiome thereof) prior to exposure to the composition or fermentate(s). In some embodiments, the abundance of Neisseria canis in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Neisseria canis in the oral cavity (or microbiome thereof) prior to exposure to the composition or fermentate(s).

In some embodiments, the abundance of Bergeyella zoohelcum in the oral cavity (or microbiome thereof) is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Bergeyella zoohelcum in the oral cavity (or microbiome thereof) prior to exposure to the composition or fermentate(s). In some embodiments, the abundance of Bergeyella zoohelcum in the oral cavity (or microbiome thereof) is reduced by at least by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Bergeyella zoohelcum in the oral cavity (or microbiome thereof) prior to exposure to the composition or fermentate(s).

In some embodiments, a composition reduces the level of gum inflammation in a subject. In some embodiments, a fermentate reduces the level of gum inflammation in a subject. In some embodiments, a fermentate is produced by a bacterial strain that reduces the level of gum inflammation in a subject. In some embodiments, the level of gum inflammation is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the level of gum inflammation in the oral cavity prior to exposure to the composition or fermentate.

In some embodiments, a composition reduces the level of gingivitis in an oral cavity of a subject. In some embodiments, a fermentate reduces the level of gingivitis in an oral cavity of a subject. In some embodiments, a fermentate is produced by a bacterial strain that reduces the level of gingivitis in an oral cavity of a subject. In some embodiments, the level of gingivitis is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the level of gingivitis in the oral cavity prior to exposure to the composition or fermentate.

In some embodiments, a composition reduces the level of plaque in an oral cavity of a subject. In some embodiments, a fermentate reduces the level of plaque in an oral cavity of a subject. In some embodiments, a fermentate is produced by a bacterial strain that reduces the level of plaque in an oral cavity of a subject. In some embodiments, the level of plaque is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the level of plaque in the oral cavity prior to exposure to the composition or fermentate.

In some embodiments, a composition reduces the level of tartar in an oral cavity of a subject. In some embodiments, a fermentate reduces the level of tartar in an oral cavity of a subject. In some embodiments, a fermentate is produced by a bacterial strain that reduces the level of tartar in an oral cavity of a subject. In some embodiments, the level of tartar is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the level of tartar in the oral cavity prior to exposure to the composition or fermentate.

In some embodiments, a composition reduces the level of dental calculus accumulation in an oral cavity of a subject. In some embodiments, a fermentate reduces the level of dental calculus accumulation in an oral cavity of a subject. In some embodiments, a fermentate is produced by a bacterial strain that reduces the level of dental calculus accumulation in an oral cavity of a subject. In some embodiments, the level of dental calculus accumulation is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the level of dental calculus accumulation in the oral cavity prior to exposure to the composition or fermentate.

In some embodiments, a composition disrupts a biofilm of one or more oral microbes. In some embodiments, a fermentate disrupts a biofilm of one or more oral microbes. In some embodiments, a fermentate is produced by a bacterial strain that disrupts a biofilm of one or more oral microbes. In some embodiments, the oral microbe is a canine oral microbe. In some embodiments, the canine oral microbe is Neisseria canis. In some embodiments, the canine oral microbe is Bergeyella zoohelcum. In some embodiments, the composition reduces the surface area of an oral microbe biofilm by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In some embodiments, the composition reduces the surface area of an oral microbe biofilm by 5-10%, 10-20%, 20-30%, 30-40%, 40-50%, 50-60%, 60-70%, 70-80%, 80-90%, or 90-100%. In some embodiments, the composition reduces the surface area of an oral microbe biofilm by 5-95%, 5-90%, 5-80%, 5-70%, 5-60%, 5-50%, 5-40%, 5-30%, 5-25%, 5-20%, 5-15%, or 5-10%. In some embodiments, the composition reduces the surface area of an oral microbe biofilm by 5-5 99%, 10-99%, 15-99%, 20-99%, 25-99%, 30-99%, 40-99%, 50-99%, 60-99%, 70-99%, 80-99%, or 90-99%.

### Enumerated Embodiments

1. A postbiotic composition comprising
(a) a fermentate of a first bacterial strain comprising a 16S rDNA sequence with at least 97% sequence identity to the nucleic acid sequence of any one of SEQ ID NOs: 1-4; and
(b) one or more additional fermentates,
wherein each of the one or more additional fermentates are of a bacterial strain comprising a 16S rDNA sequence with at least 97% sequence identity to the nucleic acid sequence of any one of SEQ ID NO: 5-9.
   2. The composition of Embodiment 1, wherein the bacterial strain of (a) is capable of disrupting a biofilm of one or more canine oral microbes.
   3. The composition of Embodiment 1 or 2, wherein the bacterial strain of (b) is capable of
(i) producing one or more compounds able to reduce the abundance of one or more volatile sulfur compounds (VSCs);
(ii) producing one or more antioxidants; and/or
(iii) enhancing a function of one or more bacterial strains.
   4. The composition of Embodiment 3, wherein the antioxidant is selected from the group consisting of a vitamin, an organic acid, a polyphenol, an indole-containing compound, and a phenol.
   5. The composition of Embodiment 4, the antioxidant is reactive to the ferric ion-reducing antioxidant power assay.
   6. A postbiotic composition comprising
(a) a first fermentate of a first bacterial strain comprising a 16S rDNA sequence with at least 97% sequence identity to the nucleic acid sequence of any one of SEQ ID NOs: 1-4; and
(b) a second fermentate of a second bacterial strain comprising a 16S rDNA sequence with at least 97% sequence identity to the nucleic acid sequence of SEQ ID NO: 5.
   7. The composition of Embodiment 6, further comprising:
(c) a third fermentate of a third bacterial strain comprising a 16s rDNA sequence with at least 97% sequence identity to the nucleic acid sequence of any one of SEQ ID NOs: 6-9.
   8. The composition of Embodiment 7, wherein
(a) the first bacterial strain comprises a 16S rDNA sequence with at least 97% sequence identity to the nucleic acid sequence of SEQ ID NO: 1 (B. subtilis);
(b) the second bacterial strain comprises a 16S rDNA sequence with at least 97% sequence identity to the nucleic acid sequence of SEQ ID NO: 5 (Pediococcus pentosaceus); and
(c) the third bacterial strain comprises a 16S rDNA sequence with at least 97% sequence identity to the nucleic acid sequence of SEQ ID NO: 6 (Lactiplantibacillus plantarum).
   9. The composition of Embodiment 8, wherein
(a) the first bacterial strain comprises a 16S rDNA sequence with at least 97% sequence identity to the nucleic acid sequence of SEQ ID NO: 1 (B. subtilis);
(b) the second bacterial strain comprises a 16S rDNA sequence with at least 97% sequence identity to the nucleic acid sequence of SEQ ID NO: 5 (Pedicoccus pentosaceus); and
(c) the third bacterial strain comprises a 16S rDNA sequence with at least 97% sequence identity to the nucleic acid sequence of SEQ ID NO: 9 (B. subtilis).
   10. A postbiotic composition comprising
(a) a first fermentate of a first bacterial strain comprising a 16S rDNA sequence with at least 97% sequence identity to the nucleic acid sequence of any one of SEQ ID NOs: 1-4; and
(b) a second fermentate of a second bacterial strain comprising a 16S rDNA sequence with at least 97% sequence identity to the nucleic acid sequence of any one of SEQ ID NOs: 6-9.
   11. The composition of Embodiment 10, further comprising a third fermentate of a third bacterial strain comprising a 16s rDNA sequence with at least 97% sequence identity to the nucleic acid sequence of SEQ ID NO: 5.
   12. The composition of Embodiment 11, wherein
(a) the first bacterial strain comprises a 16S rDNA sequence with at least 97% sequence identity to the nucleic acid sequence of SEQ ID NO: 1 (B. subtilis);
(b) the second bacterial strain comprises a 16S rDNA sequence with at least 97% sequence identity to the nucleic acid sequence of SEQ ID NO: 5 (Lactiplantibacillus plantarum); and
(c) the third bacterial strain comprises a 16S rDNA sequence with at least 97% sequence identity to the nucleic acid sequence of SEQ ID NO: 6 (Pedicoccus pentosaceus).
   13. A postbiotic composition comprising:
(a) a fermentate of a bacterial strain that disrupts an oral biofilm; and at least one of:
(b)(i) a fermentate of a bacterial strain that produces one or more compounds able to reduce the abundance of one or more volatile sulfur compounds (VSCs),
(b)(ii) a fermentate of a bacterial strain that produces one or more antioxidants; and
(b)(iii) a fermentate of a bacterial strain that enhances a function of one or more bacterial strains.
   14. The composition of Embodiment 13, wherein the bacterial strain of (a) is selected from the group consisting of Bacillus subtilis, Bacillus amyloliquefaciens, Bacillus licheniformis, and Bacillus pumilus.
   15. The composition of Embodiment 13 or 14, wherein the bacterial strain of (b)(i) is Pediococcus pentosaceus.
   16. The composition of Embodiment 13 or 15, wherein the bacterial strain of (b)(i) is selected from the group consisting of Pediococcus pentosaceus, Bacillus subtilis, and Lactiplantibacillus plantarum.
   17. The composition of any one of Embodiments 13-16, wherein the bacterial strain of (b)(ii) is selected from the group consisting of Lactiplantibacillus plantarum, Lactobacillus delbrueckii, and Limosilactobacillus fermentum.
   18. The composition of any one of Embodiments 13-17, wherein the bacterial strain of (b)(ii) is selected from the group consisting of Pediococcus pentosaceus, Lactiplantibacillus plantarum, Lactobacillus delbrueckii, and Limosilactobacillus fermentum.
   19. The composition of any one of Embodiments 13-18, wherein the bacterial strain of (b)(iii) is selected from the group consisting of Bacillus subtilis and Lactiplantibacillus plantarum.
   20. The composition of any one of Embodiments 1-19, wherein the composition reduces the abundance of one or more volatile sulfur compounds (VSCs) in an oral cavity of a subject.
   21. The composition of any one of Embodiments 1-20, wherein the composition reduces the abundance of one or more volatile sulfur compound (VSC)-producing microorganisms in a subject.
   22. The composition of Embodiment 21, wherein the one or more VSC-producing microorganisms are selected from the group consisting of Fusobacterium nucleatum, Neisseria canis, Bergyella zoohelcum, Prevotella intermedia, Porphyromonas canigingivalis, Porphyromonas gingivalis, Tannerella forsythia, and Treponema denticola.
   23. The composition of Embodiment 21 or 22, wherein the one or more VSCs are selected from the group consisting of hydrogen sulfide, methyl mercaptan, and dimethyl sulfide.
   24. The composition of any one of Embodiments 1-23, wherein the composition reduces the level of gum inflammation, gingivitis, plaque, tartar, and/or dental calculus accumulation in an oral cavity of a subject.
   25. The composition of any one of Embodiments 1-24, wherein the composition treats or prevents one or more symptom of halitosis in a subject.
   26. The composition of any one of Embodiments 1-25, wherein each of the fermentates are in powdered form.
   27. The composition of any one of Embodiments 1-26, wherein each of the fermentates are lyophilized or spray-dried.
   28. The composition of any one of Embodiments 1-27, wherein each fermentate is present in an amount of 1 to 500 mg per dose.
   29. The composition of any one of Embodiments 1-28, wherein the composition comprises a total amount of fermentate of 1 to 500 mg per dose.
   30. The composition of any one of Embodiments 1-29, further comprising a carrier.
   31. The composition of Embodiment 30, wherein the carrier is maltodextrin.
   32. The composition of Embodiment 30 or 31, wherein the carrier is a tapioca-based maltodextrin.
   33. A nutritional supplement, dental supplement, or food product comprising the composition of any one of Embodiments 1-32.
   34. The nutritional supplement, dental supplement, or food product of Embodiment 33, comprising the composition of any one of Embodiments 1-32 at an incorporation rate of 0.01% to 10% [w/w].
   35. A nutritional supplement, dental supplement, or food product of Embodiment 33 or 34, comprising the composition of any one of the preceding Embodiments at an incorporation rate of 0.1% to 2% [w/w].
   36. The nutritional supplement, dental supplement, or food product of any one of Embodiments 33-35, wherein the nutritional supplement is a canine nutritional supplement.
   37. The nutritional supplement, dental supplement, or food product of any one of Embodiments 33-36, wherein the nutritional supplement comprises a meat or animal-derived material.
   38. The nutritional supplement, dental supplement, or food product of Embodiment 37, wherein the meat or animal-derived material is beef, chicken, eggs, turkey, lamb, fish, blood marrow, and/or bone marrow.
   39. The nutritional supplement, dental supplement, or food product of any one of Embodiments 33-38, further comprising a grain.
   40. The nutritional supplement, dental supplement, or food product of Embodiment 39, wherein the grain is wheat, corn, rice, oats, and/or barley.
   41. The nutritional supplement, dental supplement, or food product of any one of Embodiments 33-39, further comprising a fiber.
   42. The nutritional supplement, dental supplement, or food product of Embodiment 41, wherein the fiber is sugar beet pulp, chicory pulp, chicory, coconut endosperm fiber, wheat fiber, fructooligosaccharides, galactooligosaccharides, and/or inulin.
   43. The nutritional supplement, dental supplement, or food product of any one of Embodiments 33-42, further comprising algae.
   44. The nutritional supplement, dental supplement, or food product of Embodiment 43, wherein the algae is selected from the group consisting of Ascophyllum nodosum, Spirulina, Chlorella, Ulva lactuca, Laminaria digitata and Fucus vesiculosus.
   45. The nutritional supplement, dental supplement, or food product of any one of Embodiments 33-44, further comprising a gelatinized starch matrix.
   46. The nutritional supplement, dental supplement, or food product of any one of Embodiments 33-45, wherein the nutritional supplement, dental supplement, or food product reduces inflammation in a subject.
   47. The nutritional supplement, dental supplement, or food product of any one of Embodiments 33-46, wherein the nutritional supplement, dental supplement, or food product treats or prevents a symptom associated with halitosis in a subject.
   48. The nutritional supplement, dental supplement, or food product of any one of Embodiments 33-47, wherein the dental supplement is an extruded or injection-molded dental stick.
   49. The nutritional supplement, dental supplement, or food product of any one of Embodiments 33-47, wherein the dental supplement is an extruded or injection-molded dental chew.
   50. The nutritional supplement, dental supplement, or food product of any one of Embodiments 33-47, wherein the nutritional supplement or dental supplement is a chew having a soft interior.
   51. The nutritional supplement, dental supplement, or food product of any one of Embodiments 33-47, wherein the nutritional supplement is a meal topper.
   52. The nutritional supplement, dental supplement, or food product of any one of Embodiments 33-47, wherein the nutritional supplement is a water additive.
   53. The nutritional supplement, dental supplement, or food product of any one of Embodiments 33-47, wherein the food product is an extruded kibble.
   54. The nutritional supplement, dental supplement, or food product of any one of Embodiments 33-47, wherein the food product is a fresh food.
   55. The nutritional supplement, dental supplement, or food product of any one of Embodiments 33-47, wherein the nutritional supplement is a liquid spray.
   56. The nutritional supplement, dental supplement, or food product of any one of Embodiments 33-47, wherein the food product is peanut butter spread.
   57. The nutritional supplement, dental supplement, or food product of any one of Embodiments 33-47, wherein the nutritional supplement, dental supplement, or food product is soft jerky chew.
   58. An oral care product comprising the composition of any one of Embodiments 1-32.
   59. The oral care product of Embodiment 58, comprising the composition of any one of Embodiments 1-32 at an incorporation rate of 0.01% to 10% [w/w].
   60. The oral care product of Embodiment 58 or 59, comprising the composition of any one of Embodiments 1-32 at an incorporation rate of 0.1% to 2% [w/w].
   61. The oral care product of any one of Embodiments 58-60, wherein the oral care product treats or prevents a symptom associated with halitosis in a subject.
   62. An oral care product of any one of Embodiments 58-61, wherein the oral care product is a toothpaste.
   63. An oral care product of any one of Embodiments 58-61, wherein the oral care product is a mouthwash.
   64. An oral care product of any one of Embodiments 58-61, wherein the oral care product is a breath mint.
   65. An oral care product of any one of Embodiments 58-61, wherein the oral care product is a chewing gum.
   66. A method comprising administering the composition of any one of Embodiments 1-32 or the nutritional supplement, dental supplement, or food product of any one of Embodiments 33-57 or the oral care product of any one of Embodiments 58-65 to a subject.
   67. The method of Embodiment 66, wherein the administration is oral administration.
   68. The method of Embodiment 66 or 67, wherein the subject has or is at risk of having halitosis.
   69. The method of any one of Embodiments 66-68, wherein an oral microenvironment of the subject comprises one or more bacterial strains selected from Porphyromonas gingivalis, Prevotella intermedia, Porphyromonas canigingivalis, Tannerella forsythia, Treponema denticola, Fusobacterium nucleatum, Neisseria canis, and Bergeyella zoohelcum.
   70. The method of any one of Embodiments 66-69, wherein administration of the composition, nutritional supplement, dental supplement, or food product:
(i) reduces the abundance of one or more VSCs in an oral microenvironment of the subject;
(ii) reduces inflammation in the oral microenvironment of the subject;
(iii) reduces the abundance of gingivitis, plaque, tartar, and/or dental calculus in the subject;
(iv) reduces the abundance of one or more microorganisms associated with the production of VSCs;
(v) reduces one or more symptom associated with halitosis; and/or
(vi) improves oral health of the subject.
   71. The method of any one of Embodiments 66-70, wherein the subject is a carnivore.
   72. The method of any one of Embodiments 66-71, wherein the subject is a mammal.
   73. The method of any one of Embodiments 66-72, wherein the subject is a domesticated animal.
   74. The method of any one of Embodiments 66-73, wherein the subject is a bovine, swine, llama, alpaca, sheep, or goat.
   75. The method of any one of Embodiments 66-73, wherein the subject is a dog, cat, rabbit, guinea pig, hamster, or ferret.
   76. The method of Embodiment 75, wherein the subject is a dog.
   77. The method of Embodiment 66-70, wherein the subject is a human.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

Examples 1-3 and 5 describe experiments that have actually been conducted. Example 4 is a prophetic example. Where examples refer to the postbiotic composition containing fermentates of Strains 1 and 5, by the term “Canine Oral Health Postbiotic” or “(COHP)”, the skilled artisan will appreciate that the utility of such compositions is not limited to canines, as reducing abundance of volatile sulfur compounds, killing oral microbes, and/or disrupting oral biofilms is beneficial in the context of halitosis of other species, such as other domesticated animals (e.g., cats) and humans.

### Example 1: Identification of Fermentates Reducing Volatile Sulfur Compound (VSC) Abundance and Disrupting Biofilms

Bacterial strains associated with bad breath in dogs, due to production of malodorous volatile sulfur compounds (VSCs), were identified. Fusobacterium nucleatum was used as an exemplary bacterial strain the model used in experiments described in this Example due to its role in early stages of halitosis. F. nucleatum was grown for 2 days, treated with one of a panel of experimental compositions outlined in the table below, then allowed to grow for another 24 hours. VSC levels were quantified at the end of this 24-hour period.

| Composition | Contents |
|---|---|
| Negative Control | Media only |
| Positive Control | Acetic acid solution |
| Fermentate A | Fermentate of bacterial strain with 16S rDNA |
| | sequence of SEQ ID NO: 6 |
| Fermentate B | Fermentate of bacterial strain with 16S rDNA |
| | sequence of SEQ ID NO: 1 |
| Fermentate C | Fermentate of bacterial strain with 16S rDNA |
| | sequence of SEQ ID NO: 5 |
| Fermentate | Fermentate of 1:1:1 mixture of bacterial |
| A + B + C | strains having 16S rDNA sequences of SEQ |
| | ID NOs. 1, 5, and 6. |

VSC levels were assayed using a colorimetric assay based on the interaction between free sulfhydryl groups in solution and DTNB (5,5′-Dithiobis (2-nitrobenzoic acid)), which cause the solution to change color. Changes in absorption were measured and normalized to the positive and negative control groups.

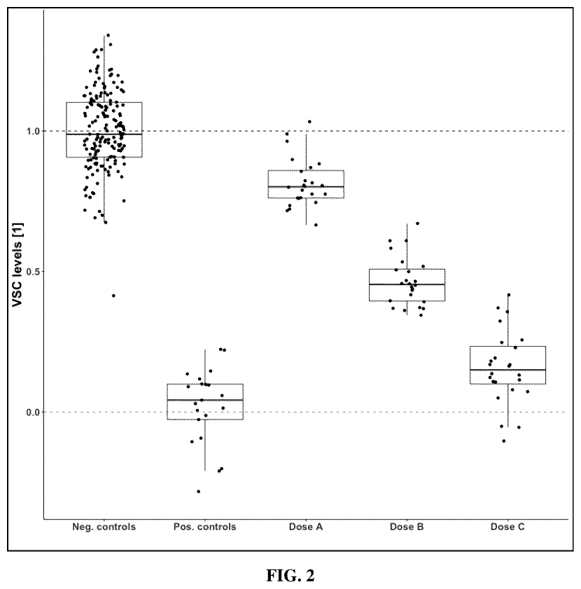

At the relevant dosage, a statistically significant reduction in VSC of 50%, as well as synergy between the strains, was observed with the fermentate of strains A, B, and C (FIG. 1). Reduction is dose-dependent, with VSCs being further increased at higher doses of fermentate (FIG. 2).

Fermentate compositions were further evaluated for their ability to disrupt biofilms of canine oral microflora. Canine oral microbiome samples were collected from dogs. Individual strains were isolated and identified through sequencing. Two canine strains were selected based on their ability to form biofilms and also produce VSCs: Neisseria canis and Bergeyella zoohelcum.

Canine oral microflora strains were grown for 24 hours in liquid media, treated with a fermentate composition (or positive control (protease K solution) or negative control (media only)), and rinsed to remove any debris. To quantify biofilm disruption, samples were labeled with DAPI, then imaged with a high-content optical microscope. Resulting images were processed to quantify the area covered in biofilm.

At the relevant dosage and treatment time (5 minutes), statistically significant biofilm disruption was observed following treatment with fermentate of a bacterial strain having a 16S rDNA sequence of SEQ ID NO: 1 (FIG. 3). While negative control (media only) resulted in a biofilm area of 54%, treatment with fermentates showed a near-complete biofilm disruption, resulting in a biofilm area of only 13%. These results indicate that fermentates have the ability of disrupt biofilm of an oral canine microbe.

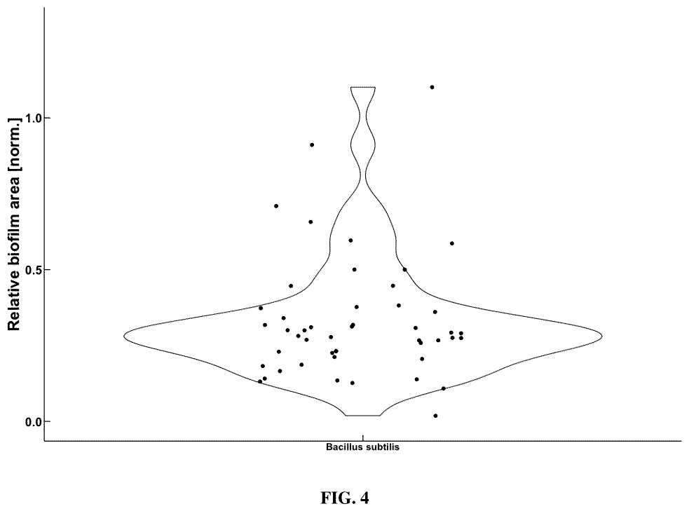

In another experiment, a biofilm-forming microbe isolated from canine oral microbiota and observed to produce VSCs was grown in microwell plates for 24 hours. Each well was treated for five minutes with a different test fermentate, and wells were then washed. Wells were labeled with DNA-staining dye DAPI and imaged using fluorescence microscopy to measure biofilm areas in each well, which were normalized to wells treated with a negative control (media only). Test materials included individual fermentates of 53 different Bacillus subtilis strains. 88% of the Bacillus subtilis fermentates achieved a statistically significant reduction in biofilm area, with 75% of fermentates reducing biofilm area by more than 50% (FIG. 4). These results support the general ability of Bacillus subtilis fermentates to disrupt canine oral microbiota biofilms.

In another experiment, microbes isolated from canine oral cavities were screened for their ability to produce VSCs in vitro. Of sixteen genera screened, five (31%) were able to produce VSCs, including Neisseria and Bergeyella.

### Example 2: In Vivo Effects of Fermentate Mixtures in Reducing VSC Abundance and Reducing Abundance of Pathogenic and VSC-Producing Canine Oral Microbe Taxa

In a clinical study, dogs received either (i) a Placebo powder, or (ii) a powder containing a mixture of a dry fermentate of a bacterial strain with 16S rDNA sequence of SEQ ID NO: 1 (Fermentate A) and of a bacterial strain with 16S rDNA sequence of SEQ ID NO: 5 (Fermentate B), termed Canine Oral Health Postbiotic (COHP). The Placebo powder was tapioca maltodextrin.

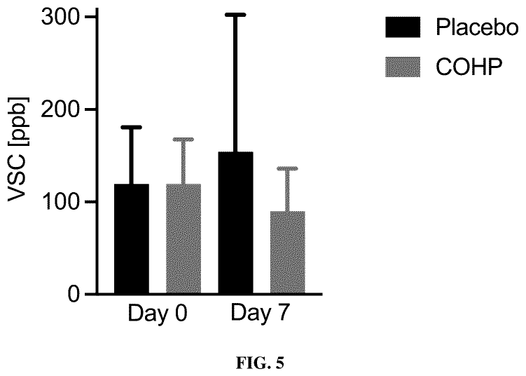

The dogs received their assigned treatment each day for 7 days. The treatment was delivered as a powder meal topper on the first meal of the day. Total VSC levels in the oral cavity were assayed using a Halimeter® PLUS instrument on Day 0 and Day 7. COHP treatment resulted in a significant decrease in VSC levels after 7 days of treatment (p=0.002), while the placebo group had no significant change in VSC levels after 7 days of treatment (p=0.455) (FIG. 5, Wilcoxon matched-pairs signed rank test, one-tailed).

In a separate study, another group of dogs received either (i) Placebo powder, or (ii) a COHP powder containing a 1:1 mixture of a dry fermentate of a bacterial strain with 16S rDNA sequence of SEQ ID NO: 1 (Fermentate A) and of a bacterial strain with 16S rDNA sequence of SEQ ID NO: 5 (Fermentate B). The Placebo powder was tapioca maltodextrin. The dogs received their assigned treatment every day for 7 days. The treatment was delivered as a powder meal topper on the first meal of the day. To quantify abundance of different oral microbiome members, oral swabs were taken from four quadrants of each subject's oral cavity on day 0 (TO) and day 7 (T7).

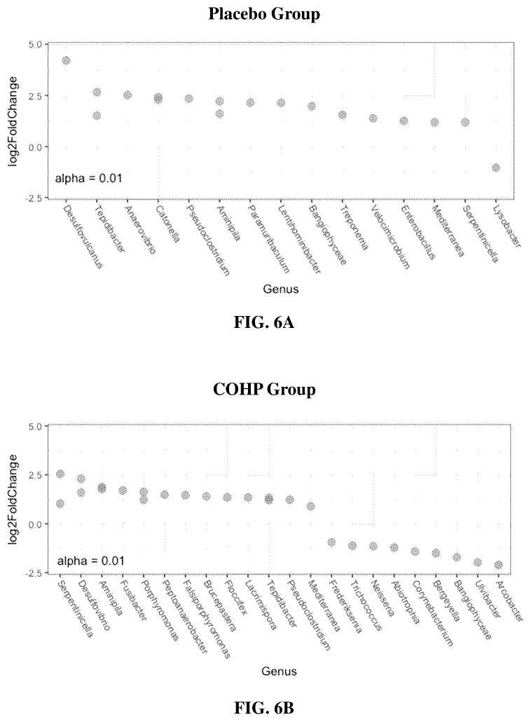

DNA was extracted with individual quadrants (biological replicates) per subject per time point being processed as separate samples. The V4 region of the 16S rRNA gene was amplified with sequencing primers and the resulting libraries were pooled and sequenced on the Illumina platform. Counts of the operational taxonomic units (OTUs) were determined for each library using USEARCH. Edgar, drive5.com. Taxa that were detected fewer than 20 times in at least 20% of samples were removed, and read counts were standardized to the median sequencing depth. Fold changes were calculated for each subject as the T7 abundance relative to the TO time point, and log 2 of the fold change for OTUs with a false discovery rate (FDR)-corrected p-value less than 0.01 was plotted (FIGS. 6A and 6B). The Wald test was used in DESeq2 for hypothesis testing to determine significance of fold changes for each OTU.

The number of OTUs that increased in abundance was similar for both groups, with 17 OTUs increasing for the control group and 18 increasing for the treatment group. Only one OTU decreased for the Placebo group, while markedly more OTUs (9) exhibited reduced abundance in the COHP treatment group, including the taxa Frederiksenia, Trichococcus, Neisseria, Abiotrophia, Corynebacterium, Bergeyella, Bangiophyceae, Ulvibacter, and Arcobacter. Neisseria and Bergeyella, in particular, were found in Example 1 to produce VSCs, and so their reduction in the Treatment group underscores the ability of COHP containing Fermentates A and B to reduce the abundance of VSC-producing microbes in the canine oral cavity.

Of the OTUs that changed in abundance in this study, many are known to be pathogenic. Known pathogenic OTUs in the Placebo group represented 23% of the total number of OTUs that increased in abundance from day 0 to day 7. Similarly, 22% of the OTUs that increased in the treatment group are known pathogens. Among the OTUs that decreased, however, the single decreased OTU in the Placebo group is not known to cause disease, but 7 of 9 (78%) of the OTUs that decreased in abundance in the Treatment group are considered pathogenic. These results thus support the ability of COHP containing Fermentates A and B to reduce the abundance of pathogenic microorganisms in the canine oral cavity, thereby improving oral health.

### Example 3: Identification of Fermentates Reducing VSC Abundance and Disrupting Biofilms with Neisseria canis

Bacterial strains associated with bad breath in dogs, due to production of malodorous volatile sulfur compounds (VSCs), were identified. Neisseria canis was used as an exemplary bacterial strain the model used in experiments described in this Example due to its ability to (i) form biofilms, and (ii) produce VSCs. N. canis was grown for 2 days in a flask, then for 1 day in a microwell plate, and finally treated with one of a panel of experimental compositions outlined in the table below for five minutes. Then, a VSC levels assessment or a biofilm disruption assessment was performed.

| Composition | Contents |
|---|---|
| Negative Control | Media only |
| Positive Control | Proteinase K solution |
| Fermentate | Fermentates of (A) a bacterial strain having a 16S |
| A + B | rDNA sequence of SEQ ID NO: 1, and (B) a bacterial |
| (COHP) | strain having a 16S rDNA sequence of SEQ ID NO: 5, |
| | combined in a 1:2.4 ratio (1 part Fermentate A + |
| | 2.4 parts Fermentate B) |

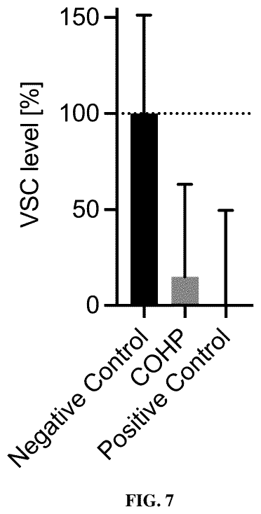

After washing with media at the end of the treatment time, samples were allowed to grow for another 24 hours. VSC levels were quantified at the end of this 24-hour period. VSC levels were assayed using a colorimetric assay based on the interaction between free sulfhydryl groups in solution and DTNB (5,5′-Dithiobis (2-nitrobenzoic acid)), which cause the solution to change color. Changes in absorption were measured and normalized to the positive and negative control groups. At the relevant dosage, a statistically significant reduction in VSCs by an average of 85% was measured with fermentate A+B (FIG. 7). The negative control (media only treatment) resulted in a normalized VSC level of 100%. The average decrease of 85% is statistically significant when compared to the negative control group (p-value: 0.0002, Dunnett's test for comparing several treatments with a control, 95% family-wise confidence level, two-sided).

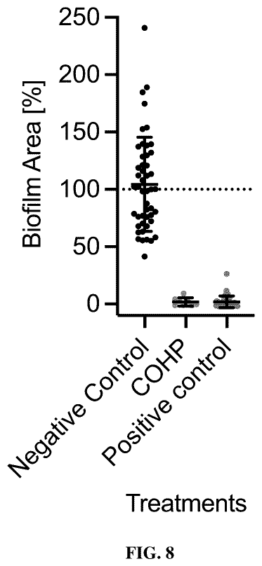

After washing with a phosphate buffer solution at the end of the treatment time, samples were labeled with DAPI, then imaged with a high-content optical microscope. Resulting images were processed to quantify the area covered in biofilm. At the relevant dosage and treatment time, COHP demonstrated a near-complete biofilm disruption with a normalized biofilm reduction of 98%, which was equivalent to that of the positive control (FIG. 8). The decrease in biofilm area due to COHP treatment is statistically significant when compared to the negative control group (p-value <0.0001, Dunnett's test for comparing several treatments with a control, 95% family-wise confidence level, two-sided).

### Example 4: Canine Clinical Study Evaluating Dental Plaque and Tartar

This Example describes a planned clinical study in canine subjects.

In a double-blind placebo-controlled clinical study, dogs receive either (i) a Placebo powder, or (ii) a powder containing a mixture of a dry fermentate of a bacterial strain with 16S rDNA sequence of SEQ ID NO: 1 (Fermentate A) and of a bacterial strain with 16S rDNA sequence of SEQ ID NO: 5 (Fermentate B). The Placebo powder is tapioca maltodextrin.

Prior to treatment, dogs receive a dental cleaning as per the Veterinary Oral Health Council protocol. Dogs receive their assigned treatment each day for 28 days. The treatment is delivered as a powder meal topper on the first meal of the day. The amount of dental plaque is assessed on the last day of treatment using Plaque Index (modified Logan & Boyce). The amount of calculus/tartar is assessed on the last day of treatment using Calculus/Tartar Index (modified Warrick & Gorrel). The health of gums is assessed on the last day of treatment and prior to treatment using Gingival Index (Loe & Silness). The amount of oral VSC is quantified on the last day of treatment using a Halimeter® Plus device.

### Example 5: Evaluation of Changes in VSC Abundance and Oral Microbes in Two Canine Clinical Studies

In a first double-blind placebo-controlled clinical study, dogs received either (i) a Placebo powder, or (ii) a powder containing a mixture of a dry fermentate of a bacterial strain with 16S rDNA sequence of SEQ ID NO: 1 (Fermentate A) and of a bacterial strain with 16S rDNA sequence of SEQ ID NO: 5 (Fermentate B), termed Canine Oral Health Postbiotic (COHP). The Placebo powder was tapioca maltodextrin. The dogs received their assigned treatment each day for 14 days. The treatment was delivered as a powder meal topper on the first meal of the day. Total VSC levels in the oral cavity were assayed using a Halimeter® PLUS instrument on Day 0, Day 7, and Day 14.

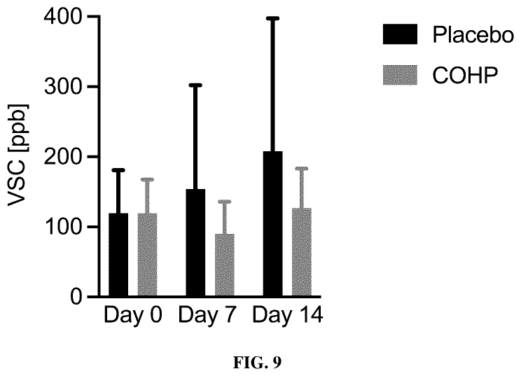

After 7 days of treatment, COHP reduced VSC levels from baseline (median change=−28%; p=0.002, paired Wilcoxon one-tailed test, FIG. 9), while the placebo did not (p=0.455, paired Wilcoxon one-tailed test). Additionally, on Days 7 and 14, VSC levels in the COHP group were lowered by 27% compared to the placebo group (p=0.004, mixed linear regression of log-transformed data, FIG. 9).

In the same study, two samples of the oral microbiome were taken by running a swab along the gum line for 5-10 seconds on the left and right side of the mouth. DNA was extracted from the oral swabs of each side individually and the V4 region of the 16S rRNA gene was amplified and sequenced on the Illumina platform. Counts of the zero-radius operational taxonomic units (ZOTUs, or taxa), which distinguish exact sequence variants, were determined for each library using USEARCH and were standardized to the sequencing depth and used for analysis. The DESeq2 package was utilized to detect taxa with significant differences across groups.

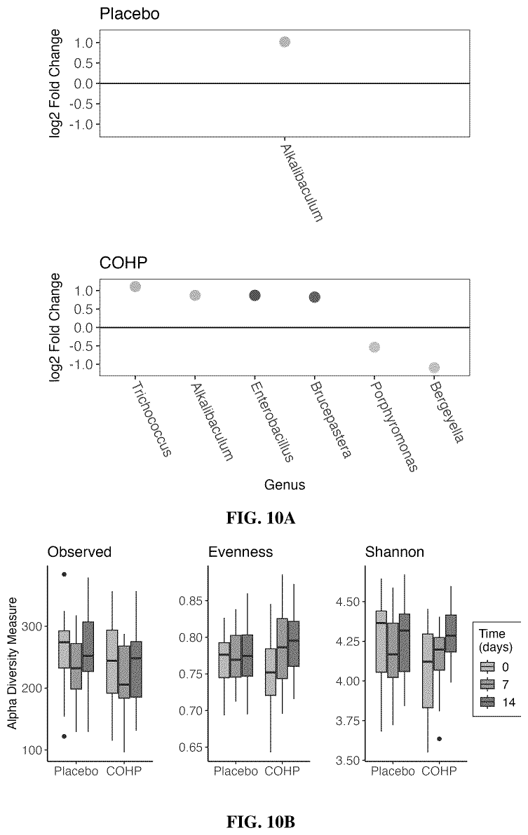

COHP reduced abundance of taxa that are associated with periodontal disease, produce VSCs, and form biofilms. COHP decreased the abundance of two taxa (FIG. 10A): Porphyromonas cangingivalis, the most prevalent oral pathogen in dogs with early periodontal disease, and Bergeyella zoohelcum, a biofilm-former & VSC producer. Holcombe et al., 2014. PloS One. 9 (12): e113744. A decrease in the abundance of taxa in the Bergeyella genus was also observed during the in vivo study described in Example 2. In addition, COHP increased microbiome community evenness on Day 7 (p=0.027, paired Wilcoxon two-tailed test, 4.6% increase, FIG. 10B; middle panel) and Day 14 (p=0.003, paired Wilcoxon two-tailed test, 5.8% increase). COHP did not increase the number of unique taxa present (FIG. 10B; left panel) in the oral microbiome. Evenness and diversity is generally associated with microbiome robustness, resilience and health.

In a second double-blind placebo-controlled clinical study, another group of dogs received either (i) Placebo powder, or (ii) a COHP powder containing a 1:1 mixture of a dry fermentate of a bacterial strain with 16S rDNA sequence of SEQ ID NO: 1 (Fermentate A) and of a bacterial strain with 16S rDNA sequence of SEQ ID NO: 5 (Fermentate B). The Placebo powder was tapioca maltodextrin. The dogs received their assigned treatment each day for 14 days. The treatment was delivered as a powder meal topper on the first meal of the day. Total VSC levels in the oral cavity were assayed using a Halimeter® PLUS instrument on Day 0, Day 7, and Day 14 together with breath scoring. For breath scoring, a veterinary technician indicated their perception of each dog's breath by choosing a number from 1-10, with 1 indicating no bad odors present and 10 indicating a high level of malodor such that they could not tolerate the smell of the dog's breath.

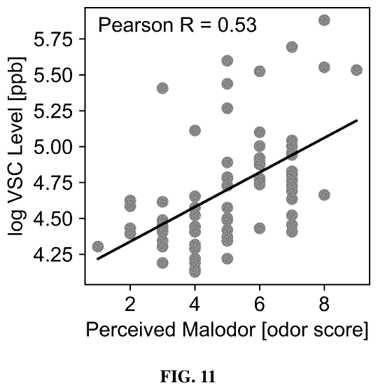

VSC levels were correlated with perceived malodor (FIG. 11). Consistent with the VSC level results, the placebo drove an increasing trend in perceived malodor at Day 14 (absolute change=+0.8 points; p=0.06, paired Wilcoxon one-tailed test), whereas perceived malodor did not change in the COHP group (absolute change=−0.3 points; p=0.3, paired Wilcoxon one-tailed test). Subsequently, COHP trended toward decreasing perceived malodor by one point more from baseline compared to the placebo group on Day 14 (p=0.06, one-tailed Mann-Whitney test on the absolute odor score difference).

What is claimed is:

1. A method of treating one or more symptoms of halitosis in a subject in need thereof, the method comprising orally administering to the subject a nutritional supplement, dental supplement, or food product comprising a bacterial fermentate mixture, the bacterial fermentate mixture comprising:
(i) a first fermentate of a first bacterial strain comprising a 16S rDNA sequence with at least 97% sequence identity to the nucleotide sequence of any one of SEQ ID NOs: 1-4; and
(ii) a second fermentate of a second bacterial strain comprising a 16S rDNA sequence with at least 97% sequence identity to the nucleotide sequence of SEQ ID NO: 5.

2. The method of claim 1, wherein the bacterial fermentate mixture comprises:
(i) a first fermentate of a first bacterial strain comprising a 16S rDNA sequence with at least 97% sequence identity to the nucleotide sequence of SEQ ID NO: 1; and
(ii) a second fermentate of a second bacterial strain comprising a 16S rDNA sequence with at least 97% sequence identity to the nucleotide sequence of SEQ ID NO: 5.

3. The method of claim 1, wherein the bacterial fermentate mixture comprises:
(i) a first fermentate of a first bacterial strain comprising a 16S rDNA sequence with at least 99% sequence identity to the nucleotide sequence of any one of SEQ ID NOs: 1-4; and
(ii) a second fermentate of a second bacterial strain comprising a 16S rDNA sequence with at least 99% sequence identity to the nucleotide sequence of SEQ ID NO: 5.

4. The method of claim 1, wherein the bacterial fermentate mixture comprises:
(i) a first fermentate of a first bacterial strain comprising a 16S rDNA sequence with at least 99% sequence identity to the nucleotide sequence of SEQ ID NO: 1; and
(ii) a second fermentate of a second bacterial strain comprising a 16S rDNA sequence with at least 99% sequence identity to the nucleotide sequence of SEQ ID NO: 5.

5. The method of claim 1, wherein the bacterial fermentate mixture consists of:
(i) a first fermentate of a first bacterial strain comprising a 16S rDNA sequence with at least 97% sequence identity to the nucleotide sequence of any one of SEQ ID NOs: 1-4; and
(ii) a second fermentate of a second bacterial strain comprising a 16S rDNA sequence with at least 97% sequence identity to the nucleotide sequence of SEQ ID NO: 5.

6. The method of claim 1, wherein the bacterial fermentate mixture consists of:
(i) a first fermentate of a first bacterial strain comprising a 16S rDNA sequence with at least 97% sequence identity to the nucleotide sequence of SEQ ID NO: 1; and
(ii) a second fermentate of a second bacterial strain comprising a 16S rDNA sequence with at least 97% sequence identity to the nucleotide sequence of SEQ ID NO: 5.

7. The method of claim 1, wherein the bacterial fermentate mixture consists of:
(i) a first fermentate of a first bacterial strain comprising a 16S rDNA sequence with at least 99% sequence identity to the nucleotide sequence of any one of SEQ ID NOs: 1-4; and
(ii) a second fermentate of a second bacterial strain comprising a 16S rDNA sequence with at least 99% sequence identity to the nucleotide sequence of SEQ ID NO: 5.

8. The method of claim 1, wherein the bacterial fermentate mixture consists of:
(i) a first fermentate of a first bacterial strain comprising a 16S rDNA sequence with at least 99% sequence identity to the nucleotide sequence of SEQ ID NO: 1; and
(ii) a second fermentate of a second bacterial strain comprising a 16S rDNA sequence with at least 99% sequence identity to the nucleotide sequence of SEQ ID NO: 5.

9. The method of claim 1, wherein the first fermentate disrupts a biofilm of one or more canine oral microbes.

10. The method of claim 1, wherein the second fermentate reduces abundance of one or more volatile sulfur compounds (VSCs).

11. The method of claim 1, wherein the nutritional supplement, dental supplement, or food product reduces abundance of one or more VSCs in an oral microenvironment in the subject.

12. The method of claim 1, wherein the nutritional supplement, dental supplement, or food product reduces, in an oral microenvironment of the subject, abundance of one or more microorganisms that produce one or more VSCs.

13. The method of claim 1, wherein the subject is a dog or cat.

14. The method of claim 1, wherein the subject is a dog.

15. The method of claim 1, wherein each of the fermentates is present in the nutritional supplement, dental supplement, or food product at an amount of 1 to 500 mg.

16. The method of claim 1, wherein the bacterial fermentate mixture is present in the nutritional supplement, dental supplement, or food product at an incorporation rate of 0.01% to 10% w/w.

17. The method of claim 1, wherein the nutritional supplement, dental supplement, or food product is in the form of a gelatinized starch matrix, extruded or injection-molded dental stick, extruded or injection-molded dental chew, chew having a soft interior, powder meal topper, water additive, dry kibble or food, wet food, frozen food, liquid spray, peanut butter spread, or soft jerky chew.

18. A method of treating one or more symptoms of halitosis in a dog in need thereof, the method comprising orally administering to the dog a nutritional supplement, dental supplement, or food product comprising a bacterial fermentate mixture, the bacterial fermentate mixture comprising:
(i) a first fermentate of a first bacterial strain comprising a 16S rDNA sequence with at least 99% sequence identity to the nucleotide sequence of SEQ ID NO: 1; and
(ii) a second fermentate of a second bacterial strain comprising a 16S rDNA sequence with at least 99% sequence identity to the nucleotide sequence of SEQ ID NO: 5.

19. The method of claim 18, wherein the first fermentate disrupts a biofilm of one or more canine oral microbes.

20. The method of claim 19, wherein the second fermentate reduces abundance of one or more volatile sulfur compounds (VSCs).
